SEQUENCE LISTING (91 sequences)

SEQ ID NO: 1 - DNA, 777 bp, synthetic construct
  source 1..777: mol_type = other DNA; note = Modified Brassica napus DNA
atgtcggatctttcaggagctgcaacccctgaatctacttatccagaaccagagattaagttgagctcaagactcagagggatatgcttttgtctcgttgctggcgtttccgccattgttctcatcgtcctgatgatcactggccatcctttcgtccttctatttgatcgttacaggaggaagttccatcacttcattgctaagctctgggcttccataagcatctacccgttttacaaaaccgacatccaaggtttggagaatctgccatcatcagacactccttgtgtgtacgtttcgaaccaccagagttttctggatatatacacacttctcagccttggccaaagctataagttcatcagcaagacagggatattcgttattcctgtcatcggttgggctatgtccatgatgggggttgttcccttgaagaggatggacccaagaagccaagtggattgcttaaaacgctgcatggaactagtgaagaagggagcttccgtctttttcttcccagagggaacgaggagtaaggatggtcggttaggtcctttcaagaaaggggcttttacgatagcagctaagacaggagttccagtggtgccaataacgctgatgggaacagggaagatcatgccgacgggtagtgaaggtatactgaatcatggggatgtgagagtgatcatccacaagccgatatatggaagcaaagctgatcttctttgcgatgaggctagaaacaagatagctgaatctatgaatctcttgagttga

SEQ ID NO: 2 - protein, 258 aa, synthetic construct
  source 1..258: note = modified Brassica napus protein
MSDLSGAATPESTYPEPEIKLSSRLRGICFCLVAGVSAIVLIVLMITGHPFVLLFDRYRRKFHHFIAKLWASISIYPFYKTDIQGLENLPSSDTPCVYVSNHQSFLDIYTLLSLGQSYKFISKTGIFVIPVIGWAMSMMGVVPLKRMDPRSQVDCLKRCMELVKKGASVFFFPEGTRSKDGRLGPFKKGAFTIAAKTGVPVVPITLMGTGKIMPTGSEGILNHGDVRVIIHKPIYGSKADLLCDEARNKIAESMNLLS

SEQ ID NO: 3 - DNA, 26 bp, synthetic construct
  misc_feature 1..26: note = Primer P1
  source 1..26: mol_type = other DNA
caccatgtcggatctttcaggagctg

SEQ ID NO: 4 - DNA, 25 bp, synthetic construct
  misc_feature 1..25: note = Primer P2
  source 1..25: mol_type = other DNA
tcaactcaagagattcatagattca

SEQ ID NO: 5 - DNA, 28 bp, synthetic construct
  misc_feature 1..28: note = Primer P3
  source 1..28: mol_type = other DNA
cggaattcatgtcggatctttcaggagc

SEQ ID NO: 6 - DNA, 32 bp, synthetic construct
  misc_feature 1..32: note = Primer P4
  source 1..32: mol_type = other DNA
gctctagatcaactcaagagattcatagattc

SEQ ID NO: 7 - DNA, 21 bp, synthetic construct
  misc_feature 1..21: note = Primer P5
  source 1..21: mol_type = other DNA
gttgattgaatcgaggaagga

SEQ ID NO: 8 - DNA, 21 bp, synthetic construct
  misc_feature 1..21: note = Primer P6
  source 1..21: mol_type = other DNA
ctttttaccacatgcaaaggg

SEQ ID NO: 9 - DNA, 20 bp, synthetic construct
  misc_feature 1..20: note = Primer P7
  source 1..20: mol_type = other DNA
ccattttggtggtcgtctat

SEQ ID NO: 10 - DNA, 21 bp, synthetic construct
  misc_feature 1..21: note = Primer P8
  source 1..21: mol_type = other DNA
cagaaaaattaaccgggtggt

SEQ ID NO: 11 - DNA, 20 bp, synthetic construct
  misc_feature 1..20: note = Primer P9
  source 1..20: mol_type = other DNA
cagctgaaaccgacgtctct

SEQ ID NO: 12 - DNA, 20 bp, synthetic construct
  misc_feature 1..20: note = Primer P10
  source 1..20: mol_type = other DNA
ggtcacgcgctctttgaatc

SEQ ID NO: 13 - DNA, 19 bp, synthetic construct
  misc_feature 1..19: note = Primer P11
  source 1..19: mol_type = other DNA
gtagcacccaaggcttcca

SEQ ID NO: 14 - DNA, 19 bp, synthetic construct
  misc_feature 1..19: note = Primer P12
  source 1..19: mol_type = other DNA
gccagtgatcatcaggacg

SEQ ID NO: 15 - DNA, 22 bp, synthetic construct
  misc_feature 1..22: note = Primer P13
  source 1..22: mol_type = other DNA
gcgtggaccgcttgctgcaact

SEQ ID NO: 16 - DNA, 20 bp, synthetic construct
  misc_feature 1..20: note = Primer P14
  source 1..20: mol_type = other DNA
ctggtgggctggagttaaga

SEQ ID NO: 17 - DNA, 20 bp, synthetic construct
  misc_feature 1..20: note = Primer P15
  source 1..20: mol_type = other DNA
agaatccatcccacaagcca

SEQ ID NO: 18 - DNA, 20 bp, synthetic construct
  misc_feature 1..20: note = Primer P16
  source 1..20: mol_type = other DNA
tgtcatcggttgggctatgt

SEQ ID NO: 19 - DNA, 20 bp, synthetic construct
  misc_feature 1..20: note = Primer P17
  source 1..20: mol_type = other DNA
catcagcgttattggcacca

SEQ ID NO: 20 - DNA, 17 bp, synthetic construct
  misc_feature 1..17: note = Primer P18
  source 1..17: mol_type = other DNA
gacgcttcatctcgtcc

SEQ ID NO: 21 - DNA, 16 bp, synthetic construct
  misc_feature 1..16: note = Primer P19
  source 1..16: mol_type = other DNA
ccacaggttgcgttag

SEQ ID NO: 22 - DNA, 16 bp, synthetic construct
  misc_feature 1..16: note = Primer P20
  source 1..16: mol_type = other DNA
tccagctaagggtgcc

SEQ ID NO: 23 - DNA, 16 bp, synthetic construct
  misc_feature 1..16: note = Primer P21
  source 1..16: mol_type = other DNA
ggtgggtactcggaga

SEQ ID NO: 24 - DNA, 17 bp, synthetic construct
  misc_feature 1..17: note = Primer P22
  source 1..17: mol_type = other DNA
gaattacccgacggaca

SEQ ID NO: 25 - DNA, 16 bp, synthetic construct
  misc_feature 1..16: note = Primer P23
  source 1..16: mol_type = other DNA
acggtctgcaatacct

SEQ ID NO: 26 - DNA, 852 bp, synthetic construct
  source 1..852: mol_type = other DNA; note = Codon optimized AGPAT1 DNA sequence
atggatttatggcctggtgcttggatgttattattattattattcttgttattgctcttcctccttcctactttgtggttctgttcaccttctgcaaagtatttctttaagatggctttctacaacggatggattcttttcttggctgttttggcaatcccagtttgtgctgtgagaggtaggaacgttgaaaacatgaagattctcagactcatgcttttgcatatcaagtacctttacggaataagagtggaagttaggggtgctcatcactttcctccatctcaaccttatgttgtggtttcaaaccaccagtcttcattggatctcttaggaatgatggaagtgctccctggtagatgtgttccaatagcaaagagggagcttttgtgggctggatcagcaggtcttgcttgctggttggctggagttatttttatcgatagaaaaaggacaggtgatgcaatctcagtgatgagtgaagttgctcaaactctcttaacacaggatgtgagagtttgggtgttccctgagggaaccaggaatcataacggtagtatgttaccatttaagagaggagctttccacctcgcagttcaagctcaggttcctatagtgccaatagttatgagttcttaccaagatttctactgtaagaaggaaagaaggttcactagtggacaatgccaagttagagtgctccctccagttccaaccgagggtttaactcctgatgatgtgccagctcttgcagatagagttaggcattctatgttgacagtgttcagagagattagtaccgatggaaggggaggtggagattatttgaagaagccaggtggaggaggatga

SEQ ID NO: 27 - DNA, 21 bp, synthetic construct
  misc_feature 1..21: note = amiRNA sequence 1
  source 1..21: mol_type = other DNA
taaagcgagttccctcgacag

SEQ ID NO: 28 - DNA, 21 bp, synthetic construct
  misc_feature 1..21: note = amiRNA sequence 2
  source 1..21: mol_type = other DNA
ttgtgcccagtgtacggactt

SEQ ID NO: 29 - DNA, 21 bp, synthetic construct
  misc_feature 1..21: note = amiRNA sequence 3
  source 1..21: mol_type = other DNA
tcaaaggcacgatgatacctg

SEQ ID NO: 30 - DNA, 21 bp, synthetic construct
  misc_feature 1..21: note = amiRNA sequence 4
  source 1..21: mol_type = other DNA
tcacttgatgtgaagatgcac

SEQ ID NO: 31 - DNA, 21 bp, synthetic construct
  misc_feature 1..21: note = amiRNA sequence 5
  source 1..21: mol_type = other DNA
ttaacagctgacacgaagcct

SEQ ID NO: 32 - DNA, 21 bp, synthetic construct
  misc_feature 1..21: note = amiRNA sequence 6
  source 1..21: mol_type = other DNA
tcacttgatgtgaacacgcac

SEQ ID NO: 33 - DNA, 1239 bp, synthetic construct
  source 1..1239: mol_type = other DNA; note = FATB DNA sequence (Arabidopsis thaliana)
atggtggccacctctgctacgtcgtcattctttcctgtaccatcttcttcacttgatcctaatggaaaaggcaataagattgggtctacgaatcttgctggactcaattctgcacctaactctggtaggatgaaggttaaaccaaacgctcaggctccacctaagattaatgggaaaaaggttggtttgcctggttctgtagatattgtaaggactgataccgagacctcatcacaccctgcgccgagaactttcatcaaccagttacctgactggagcatgcttcttgctgctataactacgattttcttagcggctgagaaacagtggatgatgcttgattggaaacctaggcgttctgacatgctggtggatccttttggtatagggagaattgttcaggatggccttgtgttccgtcagaatttttctattaggtcatatgaaataggtgctgatcgctctgcatctatagaaaccgtcatgaatcatctgcaggaaacggcgcttaatcatgttaagactgctggattgcttggagatgggtttggctctacacctgagatgtttaagaagaacttgatatgggttgtcactcgtatgcaggttgtggttgataaatatcctacttggggagatgttgttgaagtagacacctgggtcagtcagtctggaaagaatggtatgcgtcgtgattggctagttcgggactgtaatactggagaaaccttaacacgagcatcaagtgtgtgggtgatgatgaataaactgacaaggagattgtcaaagattcctgaagaggttcgaggggaaatagagccttattttgtgaattctgatcctgtccttgccgaggacagcagaaagttaacaaaaattgatgacaagactgctgactatgttcgatctggtctcactcctcgatggagtgacctagatgttaaccagcatgtgaataatgtaaagtacattgggtggatcctggagagtgctccagtgggaataatggagaggcagaagctgaaaagcatgactctggagtatcggagggaatgcgggagagacagtgtgcttcagtccctcactgcagttacgggttgcgatatcggtaacctggcaacagcgggggatgtggaatgtcagcatttgctccgactccaggatggagcggaagtggtgagaggaagaacagagtggagtagtaaaacaccaacaacaacttggggaactgcaccgtaa

SEQ ID NO: 34 - DNA, 43 bp, synthetic construct
  misc_feature 1..43: note = Primer TEF-GSY-F
  source 1..43: mol_type = other DNA
ctcgcaacaaccgattccaacaagagaccgggttggcggcgca

SEQ ID NO: 35 - DNA, 74 bp, synthetic construct
  misc_feature 1..74: note = Primer TEF-GSY-R
  source 1..74: mol_type = other DNA
ataacttcgtataatgtatgctatacgaagttataagctttgaatgattcttatactcagaaggaaatgcttaa

SEQ ID NO: 36 - DNA, 1061 bp, synthetic construct
  source 1..1061: mol_type = other DNA; note = Codon optimised version of CreLPAT
aagcttatgtctgtcctcaccaagtggctgggtctcccctctttcctgttctccgtcttcgtgttctactggtctctccccatcttcgccattctgtaccgaatccgattcgcttccctgggaaagcgaaacgacatgctcgactgggctcgagccctggtcgcctacttccgagtgaccctgctccaggctggcgagcacaccctgtacaagggcggtccctgcctgtacctctgtaaccaccgatcctgggctgacttcttcattgacgcttacctgaccgagggacgagctgctctcatgtctcgatggctggtctacttcgtgttccccgtcttctgcacctcctgtatgatcctcaagggtattgtcctgttcaagcgaggaaccattgccgacaaggaagccttcaacgcctggctggaccagaccctgggatcctctcacgtccctggactgctggtgtaccccgagggacaccgatctaccaagcctgcctccctgcctctcaagcgaggtatgctccactacgctcactctcgaaagctgcccgtgcagattgtcgtgacccgaggcaaggacgaggtcctgtccgagaagtctcagtccgtgcacttcggacgaacctgcgtcaccaccttctctaaggtgctcaagtccgctgactaccccaacttcgaggccttcttcaccgacctgcaggctacctgggactcttgttgggccgctacctacggactggaggacctcaagaacgtgcctcgattctctatgcccggacctcaggcctactcctactcctcttccatgtgggtgcagcagctcgccatcaccctcgtgtctattctggtcttcgctggagtttgttacggctcctggcgaggtctggccgctgccctggctgctaccggtgctgcccagcaggtggttgctctggtgctggctgcttgggtgggttcttccgtgctccgatccttcctgtaacgatcgtttttttttatatatatatatatatatatataactgtctagaaataaagagtatcatctttcaaaaagctt

SEQ ID NO: 37 - DNA, 935 bp, synthetic construct
  source 1..935: mol_type = other DNA; note = codon optimised version of AGPAT1
aagcttatggacctgtggcccggagcttggatgctgctcctgctcctgttcctcctcctgctgttcctcctgcccaccctgtggttctgctccccctctgctaagtacttcttcaagatggccttctacaacggttggattctgttcctggccgtcctggctattcccgtctgtgctgtgcgaggacgaaacgtggagaacatgaagatcctccgactgatgctcctgcacatcaagtacctgtacggaattcgagttgaggtccgaggcgcccaccacttccctccctcccagccttacgtcgtggtctctaaccaccagtcctctctggacctcctgggtatgatggaggtgctccctggacgatgtgtccctatcgctaagcgagagctgctctgggctggttccgctggactggcttgttggctggctggcgtcatcttcattgaccgaaagcgaaccggtgacgctatttccgtgatgtctgaggtggctcagaccctcctgacccaggacgttcgagtctgggtgttccctgagggaacccgaaaccacaacggttccatgctgcccttcaagcgaggcgccttccacctcgctgtccaggctcaggtccctattgtgcccattgtcatgtcctcttaccaggacttctactgcaagaaggagcgacgattcacctctggacagtgtcaggtccgagtgctgcctcccgtgcctaccgagggactgacccccgacgacgttcctgctctggctgaccgagtccgacactccatgctgaccgtgttccgagagatttctaccgacggtcgaggcggtggagactacctcaagaagcccggcggtggaggctaacgatcgtttttttttatatatatatatatatatatataactgtctagaaataaagagtatcatctttcaaaaagctt

SEQ ID NO: 38 - DNA, 860 bp, synthetic construct
  source 1..860: mol_type = other DNA; note = codon optimised version of PLPAT
aagcttatgtccgacctgtctggtgctgctacccccgagtccacctaccctgagcctgagatcaagctgtcctctcgactccgaggaatttgcttctgtctcgtcgccggcgtgtctgctatcgtcctgattgtgctcatgatcaccggccaccccttcgtcctgctcttcgaccgataccgacgaaagttccaccacttcatcgccaagctgtgggcttccatctctatctaccccttctacaagaccgacattcagggtctggagaacctcccctcctctgacaccccctgcgtctacgtgtccaaccaccagtctttcctggacatctacaccctgctctccctcggacagtcttacaagttcatttccaagaccggcatcttcgtcattcccgtgatcggctgggccatgtccatgatgggtgtcgtgcccctgaagcgaatggacccccgatctcaggtcgactgcctgaagcgatgtatggagctcgtcaagaagggtgcctccgtcttcttcttccccgagggaacccgatctaaggacggacgactgggccccttcaagaagggcgctttcaccattgctgctaagaccggtgtgcctgtggtgcccattaccctgatgggcaccggcaagatcatgcccaccggttccgagggaattctcaaccacggtgacgtccgagtgatcattcacaagcccatctacggatctaaggctgacctgctctgtgacgaggcccgaaacaagattgctgagtccatgaacctgctctcttaacgatcgtttttttttatatatatatatatatatatataactgtctagaaataaagagtatcatctttcaaaaagctt

SEQ ID NO: 39 - DNA, 852 bp, synthetic construct
  source 1..852: mol_type = other DNA; note = HsAGPAT1 cDNA sequence
atggatttgtggccaggggcatggatgctgctgctgctgctcttcctgctgctgctcttcctgctgcccaccctgtggttctgcagccccagtgccaagtacttcttcaagatggccttctacaatggctggatcctcttcctggctgtgctcgccatccctgtgtgtgccgtgcgaggacgcaacgtcgagaacatgaagatcttgcgtctaatgctgctccacatcaaatacctgtacgggatccgagtggaggtgcgaggggctcaccacttccctccctcgcagccctatgttgttgtctccaaccaccagagctctctcgatctgcttgggatgatggaggtactgccaggccgctgtgtgcccattgccaagcgcgagctactgtgggctggctctgccgggctggcctgctggctggcaggagtcatcttcatcgaccggaagcgcacgggggatgccatcagtgtcatgtctgaggtcgcccagaccctgctcacccaggacgtgagggtctgggtgtttcctgagggaacgagaaaccacaatggctccatgctgcccttcaaacgtggcgccttccatcttgcagtgcaggcccaggttcccattgtccccatagtcatgtcctcctaccaagacttctactgcaagaaggagcgtcgcttcacctcgggacaatgtcaggtgcgggtgctgcccccagtgcccacggaagggctgacaccagatgacgtcccagctctggctgacagagtccggcactccatgctcactgttttccgggaaatctccactgatggccggggtggtggtgactatctgaagaagcctgggggcggtgggtga

SEQ ID NO: 40 - protein, 283 aa, Homo sapiens
MDLWPGAWMLLLLLFLLLLFLLPTLWFCSPSAKYFFKMAFYNGWILFLAVLAIPVCAVRGRNVENMKILRLMLLHIKYLYGIRVEVRGAHHFPPSQPYVVVSNHQSSLDLLGMMEVLPGRCVPIAKRELLWAGSAGLACWLAGVIFIDRKRTGDAISVMSEVAQTLLTQDVRVWVFPEGTRNHNGSMLPFKRGAFHLAVQAQVPIVPIVMSSYQDFYCKKERRFTSGQCQVRVLPPVPTEGLTPDDVPALADRVRHSMLTVFREISTDGRGGGDYLKKPGGGG

SEQ ID NO: 41 - DNA, 978 bp, synthetic construct
  source 1..978: mol_type = other DNA; note = CreLPAT cDNA sequence (Chlamydomonas reinhardtii gene Cre17.g738350)
atgtccgtgctcacaaagtggctgggcctgccctcattcttgttctccgtgtttgtgttctactggagtttgcccatctttgcgatcctatatcgtatccgcttcgcctctctggggaagcgcaatgatatgctcgactgggcccgcgcgctcgtcgcctacttccgagtaacgctcctgcaggcgggggagcacacgctgtacaagggagggccatgcctgtacctgtgcaaccaccgcagctgggcggacttcttcattgacgcctacctcactgagggccgcgcggcgctcatgagcaggtggctggtgtatttcgtgttcccagtgttctgtacctcgtgcatgatcctcaaggggatagtgctgttcaagcgcggcaccattgcggataaagaggccttcaacgcctggcttgaccagaccctgggcagcagccacgtgcctgggctgctggtgtaccccgagggccaccgcagcaccaagccggccagcctgccgctgaagcgcggcatgctgcactacgcgcacagccgcaagctgccggtgcagattgtcgtgactcgcggcaaggacgaggtgctgagtgagaagtcgcagtcggtgcacttcggccgcacctgcgtcaccaccttctccaaggtgctcaagtcggccgactaccccaactttgaagccttcttcacggacctgcaggcgacctgggactcgtgctgggcagccacgtacggcctggaggatctcaaaaacgtgccgcgcttctccatgccggggccccaggcctacagctacagctccagcatgtgggtccagcagctcgccatcacgctggtcagcatcctagtctttgcgggcgtgtgctacggctcctggcgcggcttggcggcggcgctggcggcgaccggcgcggcgcagcaggtggtggcactggtgctggcggcctgggtgggcagctccgtgcttcgtagcttcctctag

SEQ ID NO: 42 - protein, 325 aa, Chlamydomonas reinhardtii
MSVLTKWLGLPSFLFSVFVFYWSLPIFAILYRIRFASLGKRNDMLDWARALVAYFRVTLLQAGEHTLYKGGPCLYLCNHRSWADFFIDAYLTEGRAALMSRWLVYFVFPVFCTSCMILKGIVLFKRGTIADKEAFNAWLDQTLGSSHVPGLLVYPEGHRSTKPASLPLKRGMLHYAHSRKLPVQIVVTRGKDEVLSEKSQSVHFGRTCVTTFSKVLKSADYPNFEAFFTDLQATWDSCWAATYGLEDLKNVPRFSMPGPQAYSYSSSMWVQQLAITLVSILVFAGVCYGSWRGLAAALAATGAAQQVVALVLAAWVGSSVLRSFL

SEQ ID NO: 43 - DNA, 1338 bp, synthetic construct
  source 1..1338: mol_type = other DNA; note = LC107611.1 Nannochloropsis sp. NIES-2145 LPAT2 mRNA for lysophosphatidate acyltransferase 2, complete cds
atgtctggtccagccgatgccaggggccctgcccgagtatccgataccattattggtgcgcccgaccgccaccatgtcggtctgaccatcctcgtcgcccttctcctgagcgccttttgcttttgcgaattcttcatgctgcccgtggcgatggcgttactgcccgtgccaggtacaacatcgaggaagttgctgcggcgctgggagggcttggtagccaaaacttggctgtcctttggagggtggctcctggaaaatgtgggtggggtcaagctcattcttacgggcgactcgatactccccggtgatcgtgtgctgattctcagcaatcataggacgagaattgattggatgtttctatggtgctgggctgcacgtttcgacctgctttcctcgtaccgagtcattctcaagtcgtctttacgcactttcccctggtggggctgggggatgagtttatgtttgtttcctttcatccaccgaggaagcaacaatcgcgacgcggatttgacccgtattgaacatatttgtcagtatttaagcgagctgggtgtgccaaactccctcattctcttcccagaaggaactgacctttcccaatctaaccaaaaacgcgatcgtgattatgctctagccaaaaacctacccatttaccataacgtgcttcacccacgttcaggcgcctttattgctagcttgacggccatgcgtcctcatttggatgctgttgtggatcttactatcggctatgtcgattacacaccaggggaaagaccttccgagctctctcttctcaagggccggctaccgcacgaggtccacataaatatgaaacgatgggacataaagacgactcccttacttcgagagaacccatcggaccgggcggaacaatttttgcgcgaatcttttgatcggaaagaggcattgctgacagccttctacagcaacaacacggacacttcaggtggtgggctcaagcaaacgtcatcattgctcgtatcctcttcctcttcacctttctccaatagcagtgcacctttaggatttccttccttctcctccccctccacctccgtcgcctcttcttacaacgagccagtgtatgtacacaatgctcactggacatacggcaaagggcttttggggacggggattgccatggcgttggtgctgatgttagcgtgctgcttgcctgctgttgcgttttgcgggtatagtatcgttgtctttttgttgttcgtgacagtgaattcggtgtgggagggcttcgataccttggaattgaatttggctgtgtggtggggcgggcaggcagggcaaacgaagcaagagtga

SEQ ID NO: 44 - protein, 445 aa, Nannochloropsis sp.
MSGPADARGPARVSDTIIGAPDRHHVGLTILVALLLSAFCFCEFFMLPVAMALLPVPGTTSRKLLRRWEGLVAKTWLSFGGWLLENVGGVKLILTGDSILPGDRVLILSNHRTRIDWMFLWCWAARFDLLSSYRVILKSSLRTFPWWGWGMSLCLFPFIHRGSNNRDADLTRIEHICQYLSELGVPNSLILFPEGTDLSQSNQKRDRDYALAKNLPIYHNVLHPRSGAFIASLTAMRPHLDAVVDLTIGYVDYTPGERPSELSLLKGRLPHEVHINMKRWDIKTTPLLRENPSDRAEQFLRESFDRKEALLTAFYSNNTDTSGGGLKQTSSLLVSSSSSPFSNSSAPLGFPSFSSPSTSVASSYNEPVYVHNAHWTYGKGLLGTGIAMALVLMLACCLPAVAFCGYSIVVFLLFVTVNSVWEGFDTLELNLAVWWGGQAGQTKQE

SEQ ID NO: 45 - DNA, 1449 bp, synthetic construct
  source 1..1449: mol_type = other DNA; note = LC107612.1 Nannochloropsis sp. NIES-2145 LPAT3 mRNA for lysophosphatidate acyltransferase 3, complete cds
atgtggggcttattatggaaacacatgaccctacgactcctcttcgtctcagtctgcttggtcacctcctccatcggcgccaacatgatggccctcgccctcttcctcgtcatccgacccttttcccgctcgctgtatcgacggttggtgtcacagtgtgtggcatgcatgtggatcgatgcactgtccttgctcctaccgggaaccaacatccacattgctgctgactctgacatgcccgacgggatcactgccggcatcgtggtggccaatcaccaatacgaaggtgattggtggttcatgctcatggtcgcccgcttcctgggcctgcatgggaacgtgaagataatagtcagggagggccttaagaagatccccctcttggggtggctggtccggcttgtcgagtacccattcatctcctcctcctggtcgctctcccgcacgaacctctttgggctgctgcggagtttcaatgcggacgacttccccgtcctcctcttccagttccccgagggcgatcgcatcgatgccaaagtccgtcagcaatcgcttgcctttgcggccaaggagcaacgcccgcacctcctccacgtcctcctcccccgtactaccggtttcaacacctgcatcgaagcgcttaggacttcccatccccccgtctatgatatgacaattgccatccctggcaccacgggccagccttcctcctcctcctcctcctcctcaccttctgctgcagctgctgcggcgtctccatctgctggtgcggcggcggcggcggcggcggcgagcaacacatcaaccaacccttcttccaaccctccctcgagcagcagcaccgatactagtccagctgctgctgctgctgctgctgctgatgccgctgctgccgagagtcatgacgcttctctctttcacaccttcctccgtttctgcaacggcgagggcccacgggacgtgcatatcagactcaagcgctactccctcaacgacgtcctggccgacccccactggctcgacaccaaatgggccgagaaagaccgcgtcttgacctacttttcccgccacgcctgctttcctgctccgctccctccccacatggccaatggtgggggcagcactcagacacacggggggagggggggccccagagggccggcgggctctcatgccgtggtgggagcagcgggtaacgcccagtatctccggtcgttcaattcccggaaattcaaagcagaaacgagttttttggcgttggcacgactgttactcacgccgttgtgccttcccttgttgttgctgatggcctgtccgctcatgactctgtatgtatgcgtgagcaccgtccgaaggtggttgggggaggagattgtgttctcgccaggaggacgggagggagggagggagggcgacgccgtttacgagtccgttaattggagcgtttga

SEQ ID NO: 46 - protein, 482 aa, Nannochloropsis sp.
MWGLLWKHMTLRLLFVSVCLVTSSIGANMMALALFLVIRPFSRSLYRRLVSQCVACMWIDALSLLLPGTNIHIAADSDMPDGITAGIVVANHQYEGDWWFMLMVARFLGLHGNVKIIVREGLKKIPLLGWLVRLVEYPFISSSWSLSRTNLFGLLRSFNADDFPVLLFQFPEGDRIDAKVRQQSLAFAAKEQRPHLLHVLLPRTTGFNTCIEALRTSHPPVYDMTIAIPGTTGQPSSSSSSSSPSAAAAAASPSAGAAAAAAAASNTSTNPSSNPPSSSSTDTSPAAAAAAAADAAAAESHDASLFHTFLRFCNGEGPRDVHIRLKRYSLNDVLADPHWLDTKWAEKDRVLTYFSRHACFPAPLPPHMANGGGSTQTHGGRGGPRGPAGSHAVVGAAGNAQYLRSFNSRKFKAETSFLALARLLLTPLCLPLLLLMACPLMTLYVCVSTVRRWLGEEIVFSPGGREGGREGDAVYESVNWSV

SEQ ID NO: 47 - DNA, 1911 bp, synthetic construct
  source 1..1911: mol_type = other DNA; note = LC107613.1 Nannochloropsis sp. NIES-2145 LPAT4 mRNA for lysophosphatidate acyltransferase 4, complete cds
atgccgacctatgcggaccctcccaggtcgcgcgcctaccagcacgaaagctcctcctacagcattgataatccgatccgcagcacccacgcctccagcccacccgactatttcaatcaaaactccctccgtgagtccccctccctccactcctcatcctcagaatccgagatggactctcagagcaatggcaatggcaggacccctcaacagcggcgaggcgacattcaaaggcagacaaacaacccttccgcctccggcacgaacggcaccccccgaggagcattcaagagcacaatcttctccatggcccacgctgtcgtcctctgctggctcgtgatggccatgtccttcggcatcaatattttgaatttcctcgtttttgtaaccgtccgtccttggtcccgcctgacggcccgaaggctcatcggtcactggtggcaacggatgtgggtctcagtaatgagctacttgctccctaagagcgagatgattctgacaggggacatcgtgcttaacactgtggattcctctcgcccggccatcatcatcgcgaatcaccaggtggatgcggatttttggtatgtctgggaagtggcgcgggcctacggaatgcatggacggttgaagatcattttgaaagccgatttggctgtggtccccatcgtgggctgggggatgaaacaatttgaattttgttttttgcaacgcaattggcagagggaccggagggcgttcacgcgcttgttgtcgtcgtttgtcgaggacgggtataagtgtgccttgctcctctttccagagggcacgaccatcaacactgaagctgtcaccaagtcccaccgcttcggccgtgagcagaagaggcctcacctcgaccattgcatcctaccgcgttccacgggctttgcggctttggtcgagacaatgtgccagtccccccatggccattctcctgttatttacgatctgaccgtcgcctatcacggttactcaggcgaagttcccacctatgagatgggctacgaccgagaacaagacatggacgtcccgaacgtgttcaaaatgatacagggacgggcatgtccaaaggttcatgtccacgtcaagacgtacgaagtcgatgaggccttggttaataatcccgagcagtggttggatgctcgatggttggaaaaagacgcgctgctggaacggtttattcagacgcagagttttaggggggagaaggagggagggacacggatcattcatcctcaggggagcttggcctcgctgctttttttgttgatgttgccgctgattatcaccgtcctccttcctgccctgctcttgatcactttggtggcctggcctgtgattcttctggcgggcacgctcaactttctgaactttgtctcgcgttcggccatgggtgtggtagtcgggagcagcagcagccgtgggtctttctccacgtcttggcatcagtcctcccaacaccggcaccagcaccagcaacgggtcagtggcaatagcaccagtagcaccagtaccagcaacagtaccagcagtagcattggtcggaaggaaggggcattgaactgcaggcgaagttctcgccgaagccccagccgtgagggatgtggccacgccaatggagaaggaccgagctgtcctctcctctctgtaagcctgccgcctctccctgccgcagctgctgctgccgctcctgctgctcctgcttcttcggctccatcttctccgctattggggagcacgccgtcgccacgccggtccccacggtggaagactccggcggggatatcccctgtagtgcagagctacgggaacaggaaggggaaggaaggaggatggggtctaatggctgcggcggccgcagcggcaacgacggaagaggaggatcggtactag

SEQ ID NO: 48 - protein, 636 aa, Nannochloropsis sp.
MPTYADPPRSRAYQHESSSYSIDNPIRSTHASSPPDYFNQNSLRESPSLHSSSSESEMDSQSNGNGRTPQQRRGDIQRQTNNPSASGTNGTPRGAFKSTIFSMAHAVVLCWLVMAMSFGINILNFLVFVTVRPWSRLTARRLIGHWWQRMWVSVMSYLLPKSEMILTGDIVLNTVDSSRPAIIIANHQVDADFWYVWEVARAYGMHGRLKIILKADLAVVPIVGWGMKQFEFCFLQRNWQRDRRAFTRLLSSFVEDGYKCALLLFPEGTTINTEAVTKSHRFGREQKRPHLDHCILPRSTGFAALVETMCQSPHGHSPVIYDLTVAYHGYSGEVPTYEMGYDREQDMDVPNVFKMIQGRACPKVHVHVKTYEVDEALVNNPEQWLDARWLEKDALLERFIQTQSFRGEKEGGTRIIHPQGSLASLLFLLMLPLIITVLLPALLLITLVAWPVILLAGTLNFLNFVSRSAMGVVVGSSSSRGSFSTSWHQSSQHRHQHQQRVSGNSTSSTSTSNSTSSSIGRKEGALNCRRSSRRSPSREGCGHANGEGPSCPLLSVSLPPLPAAAAAAAPAAPASSAPSSPLLGSTPSPRRSPRWKTPAGISPVVQSYGNRKGKEGGWGLMAAAAAAATTEEEDRY

SEQ ID NO: 49 - DNA, 678 bp, synthetic construct
  source 1..678: mol_type = other DNA; note = BAA18602.1 sll1848 [Synechocystis sp. PCC 6803] mRNA sequence
gtggattccgagattaatcatcgtggtggtttgagtgctccccgcccaagggaaacgtcacttaatttagctctctaccggggcttgaaatggggggtggtgcggccactgctccatggattgttccaggcccaggtatatggtcaggaattggtgccaacccgggggccggccttggtggtgagcaaccatgccagttattttgaccccccatttttgtcctgtgccatggcccggccggtggcctttatggccaaggaagagttatttaatgtgcccctgctgggtccagccattcgcctctatggggcctatccagtcaaacggggcagtggcgatcggggagcattgcgggccgccttgacggcgctgggggatggttggttagtgggggtctttctggagggaaccagaacaaaggatggccgcattcaccagccaaaattgggggctgccatgattgcagctaaagcccaagtgcccattattcccgtcagcctagggggagtagagcaaatttttcagcccggttccccctggccccatcctgtgcctttaactattcgcattggtaaggcgatcgcccctccagtaaagaataggaaacccgaattggaagcggttactaaagcttgccaagcccaaattcacgagatgctggatttaggcagggattag

SEQ ID NO: 50 - protein, 225 aa, Synechocystis sp.
  source 1..225: note = Strain PCC6803
MDSEINHRGGLSAPRPRETSLNLALYRGLKWGVVRPLLHGLFQAQVYGQELVPTRGPALVVSNHASYFDPPFLSCAMARPVAFMAKEELFNVPLLGPAIRLYGAYPVKRGSGDRGALRAALTALGDGWLVGVFLEGTRTKDGRIHQPKLGAAMIAAKAQVPIIPVSLGGVEQIFQPGSPWPHPVPLTIRIGKAIAPPVKNRKPELEAVTKACQAQIHEMLDLGRD

SEQ ID NO: 51 - DNA, 1170 bp, synthetic construct
  source 1..1170: mol_type = other DNA; note = Nucleotide sequence encoding ER LPAT2 of Arabidopsis thaliana
atggtgattgctgcagctgtcatcgtgcctttgggccttctcttcttcatatctggtctcgctgtcaatctctttcaggcagtttgctatgtactcattcgaccactgtctaagaacacatacagaaaaattaaccgggtggttgcagaaaccttgtggttggagcttgtatggatagttgactggtgggctggagttaagatccaagtgtttgctgataatgagaccttcaatcgaatgggcaaagaacatgctcttgtcgtttgtaatcaccgaagtgatattgattggcttgtgggatggattctggctcagcggtcaggttgcctgggaagcgcattagctgtaatgaagaagtcttccaaattccttccagtcataggctggtcaatgtggttctcggagtatctctttctggaaagaaattgggccaaggatgaaagcactctaaagtcaggtcttcagcgcttgagcgacttccctcgacctttctggttagccctttttgtggagggaactcgctttacagaagccaaacttaaagccgcacaagagtatgcagcctcctctgaattgcctatccctcgaaatgtgttgattcctcgcaccaaaggtttcgtgtcagctgttagtaatatgcgttcatttgtcccagcaatttatgatatgacagtgactattccaaaaacctctccaccacccacgatgctaagactattcaaaggacaaccttcagtggtgcatgttcacatcaagtgtcactcgatgaaagacttacctgaatcagatgacgcaattgcacagtggtgcagagatcagtttgtggctaaggatgctctgttagacaaacacatagctgcagacactttccccggtcaacaagaacagaacattggccgtcccataaagtcccttgcggtggttctatcatgggcatgcgtactaactcttggagcaataaagttcctacactgggcacaactcttttcttcatggaaaggtatcacgatatcggcgcttggtctaggtatcatcactctctgtatgcagatcctgatacgctcgtctcagtcagagcgttcgaccccagccaaagtcgtcccagccaagccaaaagacaatcaccacccagaatcatcctcccaaacagaaacggagaaggagaagtaa

SEQ ID NO: 52 - protein, 389 aa, Arabidopsis thaliana
MVIAAAVIVPLGLLFFISGLAVNLFQAVCYVLIRPLSKNTYRKINRVVAETLWLELVWIVDWWAGVKIQVFADNETFNRMGKEHALVVCNHRSDIDWLVGWILAQRSGCLGSALAVMKKSSKFLPVIGWSMWFSEYLFLERNWAKDESTLKSGLQRLSDFPRPFWLALFVEGTRFTEAKLKAAQEYAASSELPIPRNVLIPRTKGFVSAVSNMRSFVPAIYDMTVTIPKTSPPPTMLRLFKGQPSVVHVHIKCHSMKDLPESDDAIAQWCRDQFVAKDALLDKHIAADTFPGQQEQNIGRPIKSLAVVLSWACVLTLGAIKFLHWAQLFSSWKGITISALGLGIITLCMQILIRSSQSERSTPAKVVPAKPKDNHHPESSSQTETEKEK

SEQ ID NO: 53 - DNA, 1149 bp, synthetic construct
  source 1..1149: mol_type = other DNA; note = Nucleotide sequence encoding ER LPAT2 of Helianthus annuus
atggctatcgcagcagcagctgttatcgtccctattggcgtcctcttcttcgtctccggcctcatcgtcaatctcattcaggcgattatatttgtgactgtacgaccgttctcgaagagcttgtttaggcggattaacagacaggtagctgagttgttgtggctggagcttgtgtggattgttgattggtgggctggagttaaggttaacctgtacacagatgccgagaccctgaagatgatgggtaaagaacatgctcttgtgatagctaatcataaaagtgacattgattggctcattggatgggtgtttgctcagagatcaggttgtcttggtagcacattggctgtcatgaagaaatcatcgaagtttcttcccgtcattggttggtcaatgtggttttctgagtatctttttcttgagagaagttgggctaaagatgaaagtaccttgaagtcaggcctccgacgtctaaaagattaccctcaacccttttggttggccctttttgttgaagggactcgctttactaaagcaaaacttttagcagctcaagaatatgcatcttcaatgggattacctgttcccagaaatgtcttaattccaagaacaaagggatttgttacttcagtgagtgaaatgagatcatttgctcctgcaatttacgatatgacggttgcgattcccaaagattcaactccgccaacaatgctgcgcctctttaaagggcagtcgtctgtgattcacgttaaagttaagcgtcatttaatgaaggacctgccagaaacagatgaaggtgttgcacaatggtgtaaagatatttttgttgccaaggatgatatattagataaacataaagaattaaacgcctttcctgattcagaactccatgaaattggccgaccattgaagtctcttgtggtggttgtatcttgggcatgtctgcttgtactcgggatcttcaagttcctgcaatggtctaatcttttatcctcatggaaggggctcacattcactgcaattgggttggggattgttacctttttaatgcaaatcttgattcagttttcgcaatctgaacgttctacacctgcaaaagtggcccccacaaggtctagcaatggtaatgtacaagagaaactgcactga

SEQ ID NO: 54 - protein, 382 aa, Helianthus annuus
MAIAAAAVIVPIGVLFFVSGLIVNLIQAIIFVTVRPFSKSLFRRINRQVAELLWLELVWIVDWWAGVKVNLYTDAETLKMMGKEHALVIANHKSDIDWLIGWVFAQRSGCLGSTLAVMKKSSKFLPVIGWSMWFSEYLFLERSWAKDESTLKSGLRRLKDYPQPFWLALFVEGTRFTKAKLLAAQEYASSMGLPVPRNVLIPRTKGFVTSVSEMRSFAPAIYDMTVAIPKDSTPPTMLRLFKGQSSVIHVKVKRHLMKDLPETDEGVAQWCKDIFVAKDDILDKHKELNAFPDSELHEIGRPLKSLVVVVSWACLLVLGIFKFLQWSNLLSSWKGLTFTAIGLGIVTFLMQILIQFSQSERSTPAKVAPTRSSNGNVQEKLH

SEQ ID NO: 55 - DNA, 1517 bp, synthetic construct
  source 1..1517: mol_type = other DNA; note = Nucleotide sequence encoding for ER LPAT2 of Camelina sativa
ctccttataatttcaatcgctcccatttaatcgcaaagttatttttattttatttttctggttggcttattttttattttattttatgttttaaatgttctgcaacttgtcttgaaatctggggtaaaaagaaaaaagaaaaaaaaaacttcgctattggtttttctggaaatctcagaaacgattattttgggttttctatacttcgcttcctcacattagagcttcggtgttcttttttttccttttctttttctttttatttgggtgtgagggacatttttccatggtgattgctgcagctgtcatcgtgcctttgggccttctcttcttcatatctggtctcgttgtcaatctcattcaggcactttgctatgtcctcattcggccactgtctaagaacacttaccggaaaatcaaccgggtggttgctgaaaccttgtggttggagcttgtttggattgttgattggtgggctggagtaaagatccaagtgtttgctgataatgagaccttcaatcgaatgggcaaagaacacgctcttgtcgtttgtaatcaccgtagtgatattgattggcttgttggatgggttctggctcagcggtcaggttccctgggaagcgctttggctgtaatgaagaagtcttccaaattccttccagtcataggctggtcaatgtggttctcagagtatctgtttctggaaagaaattggtccaaggatgaaagcactctaaagtcaggtcttcagcgcttgagtgactttcctcgacctttctggctagccctttttgtggagggaactcgctttacagaggctaaactcaaagcagcacaagagtatgcagcctcctctgacttgcctatccctcgaaatgtgttgattcctcgcaccaaaggttttgtgtcagctgttagtaatatgcgttcatttgtcccagccatttatgatatgacagtgactattccaaaaacttctccaccgcccacgatgctaagactattcaaaggacaaccttctgtggtacatgttcacatcaagtgtcactccatgaaagacttgcctgaatcagatgacgcaattgcacagtggtgcagagatcagtttgtggctaaggatgctttgttagacaaacacatagctgcagacactttccccggtcaacaggaacagaacattggccgtcccataaagtcccttgcggtggttctatcatgggcatgcgtattaactcttggagcaattaagttcctacactgggcacaactcttttcgtcatggaaaggtatcgcgctatcggggcttgctctgggtatcatcactctcggtatgcagatcctgatacgctcgtctcagtcagagcgttcaaccccagccaaagtggttccagcaaagccaaaggaccatcacaactcagaatcatcctcccaaacagaagtggagaagcagaagtaaaaaaagtggatatcaaagatcaaacaacaaacagaagaagaaaagcgtatcagttttgtta

SEQ ID NO: 56 - protein, 389 aa, Camelina sativa
MVIAAAVIVPLGLLFFISGLVVNLIQALCYVLIRPLSKNTYRKINRVVAETLWLELVWIVDWWAGVKIQVFADNETFNRMGKEHALVVCNHRSDIDWLVGWVLAQRSGSLGSALAVMKKSSKFLPVIGWSMWFSEYLFLERNWSKDESTLKSGLQRLSDFPRPFWLALFVEGTRFTEAKLKAAQEYAASSDLPIPRNVLIPRTKGFVSAVSNMRSFVPAIYDMTVTIPKTSPPPTMLRLFKGQPSVVHVHIKCHSMKDLPESDDAIAQWCRDQFVAKDALLDKHIAADTFPGQQEQNIGRPIKSLAVVLSWACVLTLGAIKFLHWAQLFSSWKGIALSGLALGIITLGMQILIRSSQSERSTPAKVVPAKPKDHHNSESSSQTEVEKQK

SEQ ID NO: 57 - DNA, 1173 bp, synthetic construct
  source 1..1173: mol_type = other DNA; note = Nucleotide sequence encoding for ER LPAT2 of Brassica napus
atggcgatggcagcagcagtgattgtgcctttggggattctcttcttcatttctggcctcgttgtcaatctcctccaggcagtttgctatgtcctcgttcgacctatgtctaagaacacatacagaaagatcaaccgggtggttgcagaaaccttgtggttggagcttgtctggatcgttgactggtgggctggagtcaagatccaagtctttgctgatgatgagacctttaatcgaatgggcaaagaacatgctcttgtcgtttgtaatcaccgaagtgatattgattggcttgttggatggattctcgctcagaggtcaggttgcctgggaagcgcattagctgtaatgaagaagtcttccaaatttctcccagtcataggctggtcaatgtggttctccgagtatctgtttcttgaaagaaattgggcaaaggatgaaagcactttacagtcaggtcttcaacgcttgaacgacttcccacggcctttctggctagctctttttgtggagggaacccgcttcacagaggcaaaacttaaagcagcacaagagtacgcagcctcctctgagttgcctgtccctcgaaatgtgttgattcctcgcaccaaaggttttgtgtcagctgttagtaacatgcgttcatttgtgccagccatatatgatatgaccgtggctattccaaaaacttctccacccccaacgatgctaagactattcaaaggacaaccttctgtggtgcatgttcacatcaagtgtcactcgatgaaggacttgcctgaaccagaagacgaaattgcacagtggtgcagagatcagtttgtggctaaggatgcactgttagacaaacacatagctgcagacactttccccggtcagaaagaacagaacattggccgtcccataaagtctcttgcagtggttgtatcatgggcatgcctactaactcttggagcaatgaagttcttacactggtcaaacctcttttcttcgtggaaaggcatcgcattatcagcctttggtctaggcatcatcactctctgtatgcagatcctgatccgctcctctcagtcggagcgttcaacacctgccaaagtcgctccagccaagccaaaggacaatcaccagtcaggaccatcctcccaaacagaagtggaggagaagcagaagtaa

SEQ ID NO: 58 - protein, 390 aa, Brassica napus
MAMAAAVIVPLGILFFISGLVVNLLQAVCYVLVRPMSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLQSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPEPEDEIAQWCRDQFVAKDALLDKHIAADTFPGQKEQNIGRPIKSLAVVVSWACLLTLGAMKFLHWSNLFSSWKGIALSAFGLGIITLCMQILIRSSQSERSTPAKVAPAKPKDNHQSGPSSQTEVEEKQK

SEQ ID NO: 59 - DNA, 1152 bp, synthetic construct
  source 1..1152: mol_type = other DNA; note = Nucleotide sequence encoding ER LPAT2 of Glycine max
atgactgcagttgtggtcgtcccattgggccttctcttcttcgcttccggcctcatcgttaacctcattcaggcaatatgctacgtggttgtgcggccggtgtcgaagaatctgtaccggcggatgaacagggtggtggcggagctgctctggctggagctcgtgtggattattgattggtgggccggtgttaaggttcaagtattcacagatcctgaaacctttcgttcaatgggtaaagagcatgctcttgttatctccaatcacagaagtgacattgattggcttgttggatgggttttagctcagcgttcaggttgccttggcagcactcttgctgtgatgaagaaatcatcgaagtttctaccggtcattggttggtcaatgtggttttctgaatatcttttcctggagagaagttgggccaaggatgaacgcacattaaagtcaggcctacagcaactgagggatttccctcttcccttttggttggctctctttgtagaaggaactcgctttacacaggccaaactattagctgctcaggagtatgcagcctcagctggattgcctgttccaagaaatgttttgattccaagaactaagggttttgtttcagcagtaaaccatatgcgctcttttgttcctgccatttatgatgtaactgtggcaattcccaagagttcacctgctcctacaatgctaagactcttcagagggaaatcttcagtggttcatgtgcatattaagcggcatgcaatgaaggatttgccagaagaagatgaagctgttgctcaatggtgtcgagatatgtttgtggctaaggatacattgttagacaaacatatagctgaggacacattcagtgatcaagagctgcaggataccggtcgacccataaagtctctagtggttgtcatatcgtgggcatgtgtcgttgttatgggggtcgtaaagttcctccaatggtcatcactactatcctcctggaagggtgttgcattttcagcatttggtttgggagttgttactctactcatgcacatcttgatcatgttctcacaatctgagcgttcaacccctaccaaggttgcccctgcaaagtccaagaatagcgaacaactggaggctagggataacaaacaagactag

SEQ ID NO: 60 - protein, 383 aa, Glycine max
MTAVVVVPLGLLFFASGLIVNLIQAICYVVVRPVSKNLYRRMNRVVAELLWLELVWIIDWWAGVKVQVFTDPETFRSMGKEHALVISNHRSDIDWLVGWVLAQRSGCLGSTLAVMKKSSKFLPVIGWSMWFSEYLFLERSWAKDERTLKSGLQQLRDFPLPFWLALFVEGTRFTQAKLLAAQEYAASAGLPVPRNVLIPRTKGFVSAVNHMRSFVPAIYDVTVAIPKSSPAPTMLRLFRGKSSVVHVHIKRHAMKDLPEEDEAVAQWCRDMFVAKDTLLDKHIAEDTFSDQELQDTGRPIKSLVVVISWACVVVMGVVKFLQWSSLLSSWKGVAFSAFGLGVVTLLMHILIMFSQSERSTPTKVAPAKSKNSEQLEARDNKQD

SEQ ID NO: 61 - DNA, 849 bp, synthetic construct
  source 1..849: mol_type = other DNA; note = Nucleotide sequence encoding for ER LPAT of Yarrowia lipolytica
atgtccgttgcatccaagctcgtcttctacgtccgcgccgccatcgccgtggtcatctttgccgcctgtgccacctacggcgtgctggcgtccaccattctcaccgccatcggcaagcagggcctggcccaatggaccgttgccagagccttctactactcggtgcgcatcttcctgggtatcagcatcaagctgcgtagccggcaggtgaccggaaccgccggtctggatgcctccaagatccaggtcgccaacaccaccaagcccattgacgacatcaccaaacacctgccccgaccatgcattctgatttccaaccaccagaacgaaatggacattctggtgctcggtcgcatcttcccccagtactgctccgtcaccgccaaaaaggccctcaagtggtaccctctgctgggccagttcatggcgctgtccggcaccatcttcctggaccgaaaggaccgaaccaagtccgtgcagaccctcggcggcgccgtcaagaccatccagagcggcaacggaggcaagggccagagcgtcttcatgttccccgagggaacccgatcctactccaaggacgtcggcatcatgcccttcaagaagggctgtttccacctggcggtccagtcgggcgctcccattgtccccgtggtggtccagaacacctcccgaatgttttctttcggccgaggcaagctggacgccggagagatccttgtcgacgtcctgagccccattgagaccaagggtctggacgccagcaacgtcgacgctctcatggccaccacttataaggccatgtgcgagactgccgaccagattggctacgctggccagaagactcagtag

SEQ ID NO: 62 - protein, 282 aa, Yarrowia lipolytica
MSVASKLVFYVRAAIAVVIFAACATYGVLASTILTAIGKQGLAQWTVARAFYYSVRIFLGISIKLRSRQVTGTAGLDASKIQVANTTKPIDDITKHLPRPCILISNHQNEMDILVLGRIFPQYCSVTAKKALKWYPLLGQFMALSGTIFLDRKDRTKSVQTLGGAVKTIQSGNGGKGQSVFMFPEGTRSYSKDVGIMPFKKGCFHLAVQSGAPIVPVVVQNTSRMFSFGRGKLDAGEILVDVLSPIETKGLDASNVDALMATTYKAMCETADQIGYAGQKTQ

SEQ ID NO: 63 - DNA, 906 bp, synthetic construct
  source 1..906: mol_type = other DNA; note = Nucleotide sequence encoding for PDCT of Arabidopsis thaliana
atgtcagccgccgcagctgaaaccgacgtctctctccgtcgcagatctaactctcttaacggaaaccacactaacggcgtcgccattgacggaaccctagacaacaacaaccgtcgcgtcggagatacaaacactcacatggatatatctgctaagaaaactgacaacggctacgccaatggtgtcggaggaggaggatggagaagcaaagcgtcgttcacgacgtggacggcgcgtgatatcgtctacgtggtgagataccattggataccgtgcatgttcgctgccggacttctgttcttcatgggcgtggagtacacgcttcagatgattcccgcgagatctgagccgttcgatcttgggtttgtggtcacgcgctctttgaatcgcgtattagcatcttcaccggatcttaacactgttttagccgcactaaacacggtgttcgtagggatgcaaacaacgtatattgtatggacatggttagtggaaggacgagcacgagccaccatcgcggctttattcatgttcacttgtcgcggcattctcggctactctactcagcttcctctccctcaggactttctaggatcaggggttgattttccggtgggaaatgtctctttcttcctcttcttctctggccatgtcgccggctcgatgatcgcatcattggacatgagaagaatgcagaggttgagacttgcaatggtctttgacatcctcaatgtattacagtcgatcagactgctcggtacaagaggacattacacaatcgaccttgcggttggagttggcgctgggattctcttcgactcattggccggaaagtacgaagagatgatgagcaagagacatttaggcactggttttagtttgatttcgaaagactctctagtcaattaa

SEQ ID NO: 64 - protein, 301 aa, Arabidopsis thaliana
MSAAAAETDVSLRRRSNSLNGNHTNGVAIDGTLDNNNRRVGDTNTHMDISAKKTDNGYANGVGGGGWRSKASFTTWTARDIVYVVRYHWIPCMFAAGLLFFMGVEYTLQMIPARSEPFDLGFVVTRSLNRVLASSPDLNTVLAALNTVFVGMQTTYIVWTWLVEGRARATIAALFMFTCRGILGYSTQLPLPQDFLGSGVDFPVGNVSFFLFFSGHVAGSMIASLDMRRMQRLRLAMVFDILNVLQSIRLLGTRGHYTIDLAVGVGAGILFDSLAGKYEEMMSKRHLGTGFSLISKDSLVN

SEQ ID NO: 65 - DNA, 840 bp, synthetic construct
  source 1..840: mol_type = other DNA; note = Nucleotide sequence encoding for PDCT of Helianthus annuus
atggggacaatcacaatgaacgccgataagatccaccaacgttccataccacaaacaaccaccctcctcaaaacatcaaacaacgtttccattaacaatttcaatctcaaaaagaccaacaattctacgaccacatggggatcgctagcggacgcgtcgtttttacaatggacaacatcggatgtgttcggtttgtttaagtaccaccccgtcccgtgtttctttgctgtttcgttactgttttttatgggtgtggagtatacgctgcggatgataccgccctcgtcacccccgtttgatattgggttcgttgccactgcgtatttgcatcgtgtcctcgtcgctagtccgacgctaaacacagttctcgcagggcttaatacggtgtttgtggggatgcaaacaacctatataatagggacgtgggtcattgaagggcggccgagagccacagtcgcggcgctgttcatgtttacttgcagagggattttgggttattcgacacagcttcctttgcctgagggattcttgggttcgggagcagactttccggtaggcaatgtgtccttcttcttgttttattctggtcatgtagcaggatcagtaattgcatcgtttgatatgaagagaatgcataggtgggagttggcctttctattcgacaccttgaaccttttgcaggtggtgcggttactaagcacaaggggacactacaccattgacttagccgtcggagtaggtgccggcatgttgtttgattccctcgccggaaaatacatgaagaaggctatggctgaaggaggtgatgtcttgtatacaaacctacctcacttatcttaa

SEQ ID NO: 66 - protein, 279 aa, Helianthus annuus
MGTITMNADKIHQRSIPQTTTLLKTSNNVSINNFNLKKTNNSTTTWGSLADASFLQWTTSDVFGLFKYHPVPCFFAVSLLFFMGVEYTLRMIPPSSPPFDIGFVATAYLHRVLVASPTLNTVLAGLNTVFVGMQTTYIIGTWVIEGRPRATVAALFMFTCRGILGYSTQLPLPEGFLGSGADFPVGNVSFFLFYSGHVAGSVIASFDMKRMHRWELAFLFDTLNLLQVVRLLSTRGHYTIDLAVGVGAGMLFDSLAGKYMKKAMAEGGDVLYTNLPHLS

SEQ ID NO: 67 - DNA, 1261 bp, synthetic construct
  source 1..1261: mol_type = other DNA; note = Nucleotide sequence encoding for PDCT of Camelina sativa
caccaaagagttagagtgtatttaactaacaaccataaattaaaaatgtccttctcaaaaataactattcatcactttcatatgacaattttctaacccaaacaagtctcctttgaaatctccgccctcgtatctcttatataacacatctctcttcaaactcccaaaaatgtccctctccgttaactctaacggccatgtcagtcgccgcagctaaacccgccgtctctcgccgtcacgtatctaacggaaacaacactaacaacgtcgccattgacgacgatcacaaccaccaacgccgcatcgtcggagataaaaacactcgaatggagatcgctgctaagaacaacggctacgccaacggtgtcatcggaggaggaggatggaggagcaaggcgtcgttcatgacgtggacgacgcgtgacgttgtctacgtggcgagacaccattggataccgtgcatgttcgctgccgggcttttgttcttcatgggggtcgagtacacgctccagatgattcccgcgagatctgagccgttcgatcttgggttcgtggccacgcgctctttgaatcgcgtcttagcatcttccccggatcttaacaccgttttagccgcactaaacacggtgttcgtattgatgcaaacaacgtatattgtatggacatggttagtggaaggacgagcacgagcaaccatctcggctttattcatgttcacgtgtcggggcattctcggctactctactcagcttcctctccctcaggattttttaggatcaggagttgattttccagtgggaaacgtctctttcttcctcttcttctcgggccacgttgccggctcgatgatcgcatcactggacatgaggagaatgcagaggtttaagctggcgagggtttttgacatcctcaatgtattacaatcgatcaggctgctcggtacaagaggacactacaccatcgaccttgcggttggagttggcgctgggattctctttgactcactggccgggaagtacgaagagatgagcagaagacaccacctaggaactggttttagtttgatatcgaaagactctctagtcaattaaatttgttttctatcaaatgtttttagttcaacacatttagttaagttgaatttagtctaatgacttatttatttcttctttataaatgcctaaatggttcattgtataccgtttgttgaacctaattatcgatctaatgttacttaaagggttgtagccttgtagttgta

SEQ ID NO: 68 - protein, 297 aa, Camelina sativa
MSVAAAKPAVSRRHVSNGNNTNNVAIDDDHNHQRRIVGDKNTRMEIAAKNNGYANGVIGGGGWRSKASFMTWTTRDVVYVARHHWIPCMFAAGLLFFMGVEYTLQMIPARSEPFDLGFVATRSLNRVLASSPDLNTVLAALNTVFVLMQTTYIVWTWLVEGRARATISALFMFTCRGILGYSTQLPLPQDFLGSGVDFPVGNVSFFLFFSGHVAGSMIASLDMRRMQRFKLARVFDILNVLQSIRLLGTRGHYTIDLAVGVGAGILFDSLAGKYEEMSRRHHLGTGFSLISKDSLVN

SEQ ID NO: 69 - DNA, 1204 bp, synthetic construct
  source 1..1204: mol_type = other DNA; note = Nucleotide sequence encoding for PDCT of Brassica napus
atgaacttttgtagcccaaacaaccttcctttccttccacaagtttcataatatctcttatataacccatatctccaagcctctcgaaatgttcttctccgttaaatctaacggtcatgtcaactacaacaatcgtccctctccgtcgcagttctaactctctcaatgaataccacactaacgcagtcgcctttgacggaatcgtcgggtcaacaagtactagccaaatggaggagattgttacgcaaaccgacgactgctacgccaatcacaacggagatggagggagaagcaaggcatcgtttatgacgtggaggatgtgcaatcctgtccaggtggcgagagtccattggataccgtgtttgctagcggtaggagttctgttcttcacgggcgtagaggagtacatgctccagatgattccggcgagttctgagccgttcgatattggttttgtggcgacgcgctctctgtatcgactcttggcttcttcaccggatcttaataccgttttagctgctctcaacacggtgtttgtagggatgcaaacgacgtatattttatggacatggttggtggaaggacgaccacgagcgaccatctcggcttgcttcatgtttacttgtcgtggcattcttggttactctactcagctccctcttcctcaggattttctaggatcaggggtagattttccggtaggaaacgtctcgttcttcctcttctactcaggccatgtcgcagggtcgacgatagcatccttggatatgaggagaatgaagaggttgagacttgccttgctttttgacatcctcaatgtattacaatcgatcaggcttctcgggacgagaggacaatacacgatcgatctcgctgtcggagttggcgctggggttctctttgactcactggctggaaaatacgaagagatgatgagcaagagacgcaatgtaggcaatggttttagtttgatttcgtctcgctagttattaatttttgtttttttttttttatgtttttagtctggacatatttaatttagttgaaatctaatgacttaaatttgctttctttcaaaatgctctaactgacggacctaactaaatgtgtacgttattgtgtagttaccatagaggtttcgtattgtcttgagcctgatattttgattttagagctcgtttatacggtagctaataataaaaaa

SEQ ID NO: 70 - protein, 289 aa, Brassica napus
MSTTTIVPLRRSSNSLNEYHTNAVAFDGIVGSTSTSQMEEIVTQTDDCYANHNGDGGRSKASFMTWRMCNPVQVARVHWIPCLLAVGVLFFTGVEEYMLQMIPASSEPFDIGFVATRSLYRLLASSPDLNTVLAALNTVFVGMQTTYILWTWLVEGRPRATISACFMFTCRGILGYSTQLPLPQDFLGSGVDFPVGNVSFFLFYSGHVAGSTIASLDMRRMKRLRLALLFDILNVLQSIRLLGTRGQYTIDLAVGVGAGVLFDSLAGKYEEMMSKRRNVGNGFSLISSR

SEQ ID NO: 71 - DNA, 804 bp, synthetic construct
  source 1..804: mol_type = other DNA; note = Nucleotide sequence encoding for PDCT of Glycine max
atgaatggcggcgctgaggcctccctcaatcacaggcgcaaacaccaaacagctcccgccgacggcgctaaaggcgttaaggtagcaaacggagccatggggaagccgtcctcttccaagcactcctgcggcgcgtcgttcatgaaatggaccgtggctgacgctgtccacgtggtgacgcaccattggatgccgtgcttgttcgcattggggcttctcttcttcatggccgtggagtacacgcttctcatggtgccgccgtcgtcgccgcccttcgaccttggcttcatcgccacacgctccctccacgcgctcctcgagtcgtcgccgaatctcaacacgctcttcgccgggctcaatacggtgtttgtggggatgcaaacgagttatatcttatggacgtggctgattgaaggacgccccagagccacaatttcagcattgttcatgttcacatgccgtgggattttaggctactccacccagctcccattgcctcaggggtttttgggctcgggtgtggacttccctgttgggaacgtgtcttttttcttgtttttttctgggcacgttgcagggtcagtgattgcttcattggacatgaggaggatgcagaggtgggaactggcttggacttttgatgtgctcaatgttttgcaagctgtgaggttgctgggtacaagaggccattacactattgatttggccgtaggggttggtgctggaattctctttgattctttagctggcaagtacgaagatagcaaaaggaatggtgctctcaaacacaatttgattgcgtga

SEQ ID NO: 72 - protein, 267 aa, Glycine max
MNGGAEASLNHRRKHQTAPADGAKGVKVANGAMGKPSSSKHSCGASFMKWTVADAVHVVTHHWMPCLFALGLLFFMAVEYTLLMVPPSSPPFDLGFIATRSLHALLESSPNLNTLFAGLNTVFVGMQTSYILWTWLIEGRPRATISALFMFTCRGILGYSTQLPLPQGFLGSGVDFPVGNVSFFLFFSGHVAGSVIASLDMRRMQRWELAWTFDVLNVLQAVRLLGTRGHYTIDLAVGVGAGILFDSLAGKYEDSKRNGALKHNLIA

SEQ ID NO: 73 - protein, 412 aa, Arabidopsis thaliana
MVATSATSSFFPVPSSSLDPNGKGNKIGSTNLAGLNSAPNSGRMKVKPNAQAPPKINGKKVGLPGSVDIVRTDTETSSHPAPRTFINQLPDWSMLLAAITTIFLAAEKQWMMLDWKPRRSDMLVDPFGIGRIVQDGLVFRQNFSIRSYEIGADRSASIETVMNHLQETALNHVKTAGLLGDGFGSTPEMFKKNLIWVVTRMQVVVDKYPTWGDVVEVDTWVSQSGKNGMRRDWLVRDCNTGETLTRASSVWVMMNKLTRRLSKIPEEVRGEIEPYFVNSDPVLAEDSRKLTKIDDKTADYVRSGLTPRWSDLDVNQHVNNVKYIGWILESAPVGIMERQKLKSMTLEYRRECGRDSVLQSLTAVTGCDIGNLATAGDVECQHLLRLQDGAEVVRGRTEWSSKTPTTTWGTAP

SEQ ID NO: 74 - DNA, 24 bp, synthetic construct
  misc_feature 1..24: note = AGPAT1 Primer 1
  source 1..24: mol_type = other DNA
caccatggatttatggcctggtgc

SEQ ID NO: 75 - DNA, 18 bp, synthetic construct
  misc_feature 1..18: note = AGPAT1 Primer 2
  source 1..18: mol_type = other DNA
tcatcctcctccacctgg

SEQ ID NO: 76 - DNA, 28 bp, synthetic construct
  misc_feature 1..28: note = AGPAT1 Primer 3
  source 1..28: mol_type = other DNA
cggaattcatggatttatggcctggtgc

SEQ ID NO: 77 - DNA, 26 bp, synthetic construct
  misc_feature 1..26: note = AGPAT1 Primer 4
  source 1..26: mol_type = other DNA
gctctagatcatcctcctccacctgg

SEQ ID NO: 78 - DNA, 1626 bp, synthetic construct
  source 1..1626: mol_type = other DNA; note = Arabidopsis thaliana At1g74960 (FAB1) nucleotide sequence
atggtgggtgcgtcttcctcttacgcatctccgttatgtacctggtttgttgctgcttgcatgtccgtctctcacggtggaggagatagccgtcaggctgttgctcttcaatctggtgggcggagtcggcgaaggaggcagcttagcaaatgctctgtcgcttctggatccgctagcattcaggctctcgtcacttcttgtttggattttggtccttgtactcactacaacaacaacaatgcattgtcttctctctttggatcgaatagtgtttctttgaatcgaaaccagaggagattgaatcgtgctgctagctccggtggagccatggcagtgatggagatggaaaaggaagctgcggttaacaagaaaccacctacggagcagcgtcgagttgtagtgacaggcatgggagttgaaacatcattgggtcatgacccacataccttctatgagaatttgctacaaggcaacagtggtattagccagattgaaaattttgattgttctgaatttcctacgcgaattgcgggagagatcaaaagcttctcgactgaaggatgggttgctccaaaactttctaaaaggatggacaaattcatgctctatcttctcacagctggtaagaaagctttggctgatggtggggttactgatgaagtaatggcagagtttgacaaaaccaaatgtggagttttgattggctcggcaatgggaggaatgaaggtcttttacgatgctattgaagctctgagaatctcttacaagaagatgaatcctttttgtgtaccttttgcgacaacaaacatgggttctgctatgcttgccatggatctgggatggatggggccaaactattctatttcaactgcttgtgccacaagcaacttttgcattctgaattcagcaaaccacattattaaaggtgaagctgatgtaatgctctgtggtggctcagatgcagttattattccaatagggttgggaggttttgttgcatgccgggctctttcacaaaggaataatgatcccacaaaagcttcacgtccttgggataccaatcgagatggtttcgtgatgggagagggagctggagttctacttttggaagaactcgagcatgctaagaaaagaggtgcaactatctacgcagagttcctcggtgggagtttcacatgtgatgcctatcacatgaccgagcctcaccctgatggggctggtgttattctctgtattgagagagcgttagctagtgctgggatttccaaggaacaaataaattacataaatgcacatgcaacctcaacgcatgctggagatattaaggaataccaagcccttgctcactgttttggccaaaatcctgagcttaaggtaaattccacaaaatctatgattggacacttgctgggagctgctggggccgtggaggctgttgcaactgtgcaggcgatacggaccggatgggttcatccaaatatcaacctcgagaatccagacagtggagtggatacaaagctgctggtgggtcctaagaaggagagactggacattaaagcagccttgtcaaattcattcgggtttggtggtcataactccagcatcatttttgctccttacaagtga

SEQ ID NO: 79 - protein, 541 aa, Arabidopsis thaliana
MVGASSSYASPLCTWFVAACMSVSHGGGDSRQAVALQSGGRSRRRRQLSKCSVASGSASIQALVTSCLDFGPCTHYNNNNALSSLFGSNSVSLNRNQRRLNRAASSGGAMAVMEMEKEAAVNKKPPTEQRRVVVTGMGVETSLGHDPHTFYENLLQGNSGISQIENFDCSEFPTRIAGEIKSFSTEGWVAPKLSKRMDKFMLYLLTAGKKALADGGVTDEVMAEFDKTKCGVLIGSAMGGMKVFYDAIEALRISYKKMNPFCVPFATTNMGSAMLAMDLGWMGPNYSISTACATSNFCILNSANHIIKGEADVMLCGGSDAVIIPIGLGGFVACRALSQRNNDPTKASRPWDTNRDGFVMGEGAGVLLLEELEHAKKRGATIYAEFLGGSFTCDAYHMTEPHPDGAGVILCIERALASAGISKEQINYINAHATSTHAGDIKEYQALAHCFGQNPELKVNSTKSMIGHLLGAAGAVEAVATVQAIRTGWVHPNINLENPDSGVDTKLLVGPKKERLDIKAALSNSFGFGGHNSSIIFAPYK

SEQ ID NO: 80 - DNA, 1152 bp, synthetic construct
  source 1..1152: mol_type = other DNA; note = Arabidopsis thaliana At3g12120 (FAD2) nucleotide sequence
atgggtgcaggtggaagaatgccggttcctacttcttccaagaaatcggaaaccgacaccacaaagcgtgtgccgtgcgagaaaccgcctttctcggtgggagatctgaagaaagcaatcccgccgcattgtttcaaacgctcaatccctcgctctttctcctaccttatcagtgacatcattatagcctcatgcttctactacgtcgccaccaattacttctctctcctccctcagcctctctcttacttggcttggccactctattgggcctgtcaaggctgtgtcctaactggtatctgggtcatagcccacgaatgcggtcaccacgcattcagcgactaccaatggctggatgacacagttggtcttatcttccattccttcctcctcgtcccttacttctcctggaagtatagtcatcgccgtcaccattccaacactggatccctcgaaagagatgaagtatttgtcccaaagcagaaatcagcaatcaagtggtacgggaaatacctcaacaaccctcttggacgcatcatgatgttaaccgtccagtttgtcctcgggtggcccttgtacttagcctttaacgtctctggcagaccgtatgacgggttcgcttgccatttcttccccaacgctcccatctacaatgaccgagaacgcctccagatatacctctctgatgcgggtattctagccgtctgttttggtctttaccgttacgctgctgcacaagggatggcctcgatgatctgcctctacggagtaccgcttctgatagtgaatgcgttcctcgtcttgatcacttacttgcagcacactcatccctcgttgcctcactacgattcatcagagtgggactggctcaggggagctttggctaccgtagacagagactacggaatcttgaacaaggtgttccacaacattacagacacacacgtggctcatcacctgttctcgacaatgccgcattataacgcaatggaagctacaaaggcgataaagccaattctgggagactattaccagttcgatggaacaccgtggtatgtagcgatgtatagggaggcaaaggagtgtatctatgtagaaccggacagggaaggtgacaagaaaggtgtgtactggtacaacaataagttatga

SEQ ID NO: 81 - protein, 383 aa, Arabidopsis thaliana
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNTGSLERDEVFVPKQKSAIKWYGKYLNNPLGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 82 - DNA, 1521 bp, synthetic construct
  source 1..1521: mol_type = other DNA; note = Arabidopsis thaliana At4g34520 (FAE1) nucleotide sequence
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgggtggctctacgcaatgtcaaggcatcggcaaatagtccttggcaacattgcatcgatagatatccggttaaaattgattctgatttgtcaaagtcaaagactcatgtccaaaacggtcggtcctaa

SEQ ID NO: 83 - protein, 506 aa, Arabidopsis thaliana
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 84 - DNA, 1260 bp, synthetic construct
  source 1..1260: mol_type = other DNA; note = Yarrowia lipolytica YALI0B10153g (FAD2) nucleotide sequence
atggattcgaccacgcagaccaacaccggcaccggcaaggtggccgtgcagccccccacggccttcattaagcccattgagaaggtgtccgagcccgtctacgacacctttggcaacgagttcactcctccagactactctatcaaggatattctggatgccattccccaggagtgctacaagcggtcctacgttaagtcctactcgtacgtggcccgagactgcttctttatcgccgtttttgcctacatggcctacgcgtacctgcctcttattccctcggcttccggccgagctgtggcctgggccatgtactccattgtccagggtctgtttggcaccggtctgtgggttcttgcccacgagtgtggccactctgctttctccgactctaacaccgtcaacaacgtcaccggatgggttctgcactcctccatgctggtcccttactacgcctggaagctgacccactccatgcaccacaagtccactggtcacctcacccgtgatatggtgtttgtgcccaaggaccgaaaggagtttatggagaaccgaggcgcccatgactggtctgagcttgctgaggacgctcccctcatgaccctctacggcctcatcacccagcaggtgtttggatggcctctgtatctgctgtctaacgttaccggacagaagtaccccaagctcaacaaatgggctgtcaaccacttcaaccccaacgccccgctgtttgagaagaaggactggttcaacatctggatctctaacgtcggtattggtatcaccatgtccgtcatcgcatactccatcaaccgatggggcctggcttccgtcaccctctactacctgatcccctacctgtgggtcaaccactggctcgtggccatcacctacctgcagcacaccgaccccactctgccccactaccacgccgaccagtggaacttcacccgaggagccgccgccaccatcgaccgagagtttggcttcatcggctccttctgcttccatgacatcatcgagacccacgttctgcaccactacgtgtctcgaattcccttctacaacgcccgaatcgccactgagaagatcaagaaggtcatgggcaagcactaccgacacgacgacaccaacttcatcaagtctctttacactgtcgcccgaacctgccagtttgttgaaggtaaggaaggcattcagatgtttagaaacgtcaatggagtcggagttgctcctgacggcctgccttctaaaaagtag

SEQ ID NO: 85 - protein, 419 aa, Yarrowia lipolytica
MDSTTQTNTGTGKVAVQPPTAFIKPIEKVSEPVYDTFGNEFTPPDYSIKDILDAIPQECYKRSYVKSYSYVARDCFFIAVFAYMAYAYLPLIPSASGRAVAWAMYSIVQGLFGTGLWVLAHECGHSAFSDSNTVNNVTGWVLHSSMLVPYYAWKLTHSMHHKSTGHLTRDMVFVPKDRKEFMENRGAHDWSELAEDAPLMTLYGLITQQVFGWPLYLLSNVTGQKYPKLNKWAVNHFNPNAPLFEKKDWFNIWISNVGIGITMSVIAYSINRWGLASVTLYYLIPYLWVNHWLVAITYLQHTDPTLPHYHADQWNFTRGAAATIDREFGFIGSFCFHDIIETHVLHHYVSRIPFYNARIATEKIKKVMGKHYRHDDTNFIKSLYTVARTCQFVEGKEGIQMFRNVNGVGVAPDGLPSKK

SEQ ID NO: 86 - DNA, 978 bp, synthetic construct
  source 1..978: mol_type = other DNA; note = Yarrowia lipolytica YALI0B20196p (FAE1/ELO2) nucleotide sequence
atgagcgccgtccctattgaattcaacgtcccctccgtggaccgaccctttggtatctacctctgggccatctttgaccaggcctgggagaagcttttcggctggcccgcgtcctctttcattttcgtgcgaaatgaccccaacatccccttttcctctacccctcccgtgatcattgccatcattgtgtactacattgtcatctttggcggccgagaggtgatgcgaaacctgtctcccatccgactcaactggctcttccagatccacaacatcttcctcacccttctgtccggtatgctcctcctcctcctcgttgagcagctcttccccatcattgtccgacagggtatcctctacgccatctgcgactacggatcttggactcagcccattgtcttctgctactacctcaactacctgaccaagtactttgagctgatcgacaccgttttccttgtgctgcgaaagaagaagctgactttcctccacacctaccaccatggtgccactgctcttctgtgctacacccagctcattggtaagacctcggtctcttgggtccccatcacccttaacctgtttgtccacgttgtcatgtacttctactacttcctggctgcgcgaggtatccgagtgtggtggaaggagtgggtcacccggctccagatcatccagttcgttatcgatcttggatttgtctactttgcctcttacacctacttcacctctacctactggccctggatgcccaacatgggctcttgtgccggcgaggagtttgctgctatttacggctgtggtctgctgacctcttacctcttcctcttcatcgccttctacatcaactcttaccgaaagccctcttccaagggaccttccaagcctgttgttgctgtcgatggccctgttggcggcgtcaacgcccagactggtgcttctcgaggccagaccactacccgatctcgacgagcataa

SEQ ID NO: 87 - protein, 325 aa, Yarrowia lipolytica
MSAVPIEFNVPSVDRPFGIYLWAIFDQAWEKLFGWPASSFIFVRNDPNIPFSSTPPVIIAIIVYYIVIFGGREVMRNLSPIRLNWLFQIHNIFLTLLSGMLLLLLVEQLFPIIVRQGILYAICDYGSWTQPIVFCYYLNYLTKYFELIDTVFLVLRKKKLTFLHTYHHGATALLCYTQLIGKTSVSWVPITLNLFVHVVMYFYYFLAARGIRVWWKEWVTRLQIIQFVIDLGFVYFASYTYFTSTYWPWMPNMGSCAGEEFAAIYGCGLLTSYLFLFIAFYINSYRKPSSKGPSKPVVAVDGPVGGVNAQTGASRGQTTTRSRRA

SEQ ID NO: 88 - DNA, 1185 bp, synthetic construct
  source 1..1185: mol_type = other DNA; note = Yarrowia lipolytica YALI0C10989g (CPT1) nucleotide sequence
atgggcgtattcattaaacaggagcagcttccggctctcaagaagtacaagtactccgccgaggatcactcgttcatctccaacaacattctgcgccccttctggcgacagtttgtcaaaatcttccctctgtggatggcccccaacatggtgactctgttgggcttcttctttgtcattgtgaacttcatcaccatgctcattgttgatcccacccacgaccgcgagcctcccagatgggtctacctcacctacgctctgggtctgttcctttaccagacatttgatgcctgtgacggatcccatgcccgacgaactggccagagtggaccccttggagagctgtttgaccactgtgtcgacgccatgaatacctctctgattctcacggtggtggtgtccaccacccatatgggatataacatgaagctgctgattgtgcagattgccgctctcggaaacttctacctgtcgacctgggagacctaccataccggaactctgtacctttctggcttctctggtcctgttgaaggtatcttgattctggtggctcttttcgtcctcaccttcttcactggtcccaacgtgtacgctctgaccgtctacgaggctcttcccgaatccatcacttcgctgctgcctgccagcttcctggacgtcaccatcacccagatctacattggattcggagtgctgggcatggtgttcaacatctacggcgcctgcggaaacgtgatcaagtactacaacaacaagggcaagagcgctctccccgccattctcggaatcgccccctttggcatcttctacgtcggcgtctttgcctgggcccatgttgctcctctgcttctctccaagtacgccatcgtctatctgtttgccattggggctgcctttgccatgcaagtcggccagatgattcttgcccatctcgtgcttgctcccttcccccactggaacgtgctgctcttcttcccctttgtgggactggcagtgcactacattgcacccgtgtttggctgggacgccgatatcgtgtcggttaacactctcttcacctgttttggcgccaccctctccatttacgccttctttgtgcttgagatcatcgacgagatcaccaactacctcgatatctggtgtctgcgaatcaagtaccctcaggagaagaagactgagtaa

SEQ ID NO: 89 - protein, 394 aa, Yarrowia lipolytica
MGVFIKQEQLPALKKYKYSAEDHSFISNNILRPFWRQFVKIFPLWMAPNMVTLLGFFFVIVNFITMLIVDPTHDREPPRWVYLTYALGLFLYQTFDACDGSHARRTGQSGPLGELFDHCVDAMNTSLILTVVVSTTHMGYNMKLLIVQIAALGNFYLSTWETYHTGTLYLSGFSGPVEGILILVALFVLTFFTGPNVYALTVYEALPESITSLLPASFLDVTITQIYIGFGVLGMVFNIYGACGNVIKYYNNKGKSALPAILGIAPFGIFYVGVFAWAHVAPLLLSKYAIVYLFAIGAAFAMQVGQMILAHLVLAPFPHWNVLLFFPFVGLAVHYIAPVFGWDADIVSVNTLFTCFGATLSIYAFFVLEIIDEITNYLDIWCLRIKYPQEKKTE

SEQ ID NO: 90 - DNA, 1332 bp, synthetic construct
  source 1..1332: mol_type = other DNA; note = Yarrowia lipolytica YALI0E26565g (EPT1) nucleotide sequence
atgggtaaaagcccctctatgattgggacttgcggttgggttgggctgggtacaaacacatgctatgctcatcagctcagccgtgccatgggccatcaaccaacttttctcccgaccctaatcccctccacactactaatgccaggattccaccacatcaaccccgacagtctagtgcatctaaagtcgtaccagtacagaagcgtcgataaatcctacctgtctaagtacatcctgaacccatggtggacttacgcagcaaccttcatgcctgattggctggctcccaacgccatcactctcatcggtgtctccggcatgctcctgtcaatcttcttcaccgtctggtacacccccgagctcaccggcgacggtccctcctggatctacttcttctctgccttttcgctcttcttctaccagaccatggacaatattgacggcaagcaggcccgtcgaactggctcttcctctcctctgggagagctgtttgaccacggaatcgactccctcaactgcacctacggcggaattgtcaactgcggtgctgtggcccttggttccacctcctacggaggcctcatggttctgtccacctgtattggtatgtacttttctacctgggagacttactacacccacactctttacctgggagttgttaacggccccaccgaagggctagtggtggccctgtctaccatgctaatctccggcttcatgggcaccgacatctggaaggaggatgccgttgaggtgctccccttcctctccttcatggttcccgagtacctcaagctcaacgagttctgggtctgggtcgtcatgttcaccctcgtggtgctgcacgtgcctttctgcgtgtggaacgtctattgggcctgcaaggaggacgatgtgcctttctccgaggctctggtgggcctcctgccctttggcgtggccggaggagctgcctacgtgtggctgcagagtccttattccactgtgctggttgacaaccaccttgtgctatttggactgaccgcctcgtgggtctttggccggctgaccaccggtgtgattctcaaccacctcaccaagctcgagttccctctgtggaactccaccctgattcctcttctgggagccaccgttctgttctacctgctccctgccctgggtctactgccccaggacaatccccacttcgagactctgtatctgtggggcttctttgtctacgctgccgcgaactttttgacctgggctgttaacaccatcaatgtcatctgttcctaccttggcatccgatgtctgtctctgcgacccgtggacaacaagaccaactag

SEQ ID NO: 91 - protein, 443 aa, Yarrowia lipolytica
MGKSPSMIGTCGWVGLGTNTCYAHQLSRAMGHQPTFLPTLIPSTLLMPGFHHINPDSLVHLKSYQYRSVDKSYLSKYILNPWWTYAATFMPDWLAPNAITLIGVSGMLLSIFFTVWYTPELTGDGPSWIYFFSAFSLFFYQTMDNIDGKQARRTGSSSPLGELFDHGIDSLNCTYGGIVNCGAVALGSTSYGGLMVLSTCIGMYFSTWETYYTHTLYLGVVNGPTEGLVVALSTMLISGFMGTDIWKEDAVEVLPFLSFMVPEYLKLNEFWVWVVMFTLVVLHVPFCVWNVYWACKEDDVPFSEALVGLLPFGVAGGAAYVWLQSPYSTVLVDNHLVLFGLTASWVFGRLTTGVILNHLTKLEFPLWNSTLIPLLGATVLFYLLPALGLLPQDNPHFETLYLWGFFVYAAANFLTWAVNTINVICSYLGIRCLSLRPVDNKTN

(19) United States
(12) Patent Application Publication
(10) Pub. No.: US 2025/0230460 A1
(43) Pub. Date: Jul. 17, 2025

(54) NON-HUMAN ORGANISM FOR PRODUCING TRIACYLGLYCEROL

(71) Applicant: ROTHAMSTED RESEARCH LTD, Harpenden Hertfordshire (GB)

(72) Inventors: Peter EASTMOND, Harpenden Hertfordshire (GB); Henricus Egbertus Gerardus VAN ERP, Harpenden Hertfordshire (GB); Govindprasad BHUTADA, Harpenden Hertfordshire (GB)

(73) Assignee: ROTHAMSTED RESEARCH LTD, Harpenden Hertfordshire (GB)

(21) Appl. No.: 19/086,458

(22) Filed: Mar. 21, 2025

Related U.S. Application Data
(63) Continuation of application No. 17/633,390, filed on Feb. 7, 2022, now Pat. No. 12,281,317.

(30) Foreign Application Priority Data
Aug. 7, 2019 (GB) 1911317.4

(51) Int. Cl. C12N 15/82; A23L 33/115

(52) U.S. Cl. CPC C12N 15/8247; A23L 33/115

(57) ABSTRACT

Described herein are non-human organisms for producing triacylglycerol wherein the non-human organism is genetically modified to express a lysophosphatidic acid acyltransferase specific for C16:0-Coenzyme A (C16:0 LPAT) and wherein, once expressed, the C16:0 LPAT is localised in the endoplasmic reticulum. Also described are non-human organisms for producing triacylglycerol in which (a) a lysophosphatidic acid acyltransferase specific for C16:0-Coenzyme A (C16:0 LPAT) is localised in the endoplasmic reticulum; and (b) activity of native endoplasmic reticulum lysophosphatidic acid acyltransferase (ER LPAT) is suppressed or prevented. Further described are non-human organisms in which a chloroplast lysophosphatidic acid acyltransferase (LPAT) is expressed, wherein said chloroplast LPAT lacks a functional chloroplast targeting signal. The disclosure also relates to methods for extracting triacylglycerol from the organisms and the use thereof in infant formula.

### CROSS-REFERENCE TO RELATED APPLICATIONS

This application is a continuation under 35 U.S.C. § 120 of co-pending U.S. Ser. No. 17/633,390 filed Feb. 7, 2022, which is a 35 U.S.C. § 371 National Phase Entry Application of International Application No. PCT/GB2020/051875 filed Aug. 6, 2020, which designates the U.S. and claims benefit under 35 U.S.C. § 119(a) of G.B. Provisional Application No. 1911317.4 filed Aug. 7, 2019, the contents of which are incorporated herein by reference in their entireties.

### SEQUENCE LISTING

The instant application contains a Sequence Listing which has been submitted electronically in XML format and is hereby incorporated by reference in its entirety. Said XML copy, created Apr. 2, 2025, is named P16317S-PCT sequence listing 020425.xml and is 144,016 bytes in size.

The present invention relates to non-human organisms, in particular to plants, fungi and yeast, for producing triacylglycerol.

Infant formula is a manufactured food designed to substitute for human breast milk. Around half the calories in human milk are provided by fat (triacylglycerol; TAG) and in infant formula this fat is mainly sourced from plants. Although blended vegetable fats can replicate the fatty acyl composition of human milk fat (HMF), which mainly comprises palmitate (C16:0) and oleate (C18:1), the arrangement of acyl groups esterified to the glycerol backbone (i.e. the stereoisomeric structure) is profoundly different. In vegetable fats, saturated long-chain fatty acyl groups such as C16:0 occupy the outer stereospecific numbering (sn) positions (sn-1/3) and are virtually excluded from the middle (sn-2 or β) position. Whereas in HMF more than 70% of the C16:0 is present at the sn-2 position, with unsaturated fatty acyl groups (mainly C18: 1) occupying the outer sn-1/3 positions.

Multiple clinical trials on preterm and term infants have suggested that the unusual stereoisomeric structure of HMF is important for nutrient absorption in the neonatal gut. The proposed mechanism is as follows. During the intestinal phase of digestion lipases attack ingested fat at the sn-1/3 positions yielding 2-monoacylglycerols, which are easily absorbed. When unsaturated fatty acids are released from sn-1/3 positions they are also absorbed easily, but the release of long-chain saturated fatty acids such as C16:0 presents a problem. Their melting point is higher than body temperature and, at intestinal pH they are prone to form hydrated fatty acid soaps with minerals such as calcium and magnesium. The arrangement of C16:0 at the sn-1/3 positions of vegetable fats thus means that they are more poorly absorbed than HMF. There is evidence that the formation of C16:0 soaps also reduces calcium absorption, thus impairing early bone development, and accumulation of these soaps in the intestine also disrupts transit, causing infants discomfort.

To mimic the stereoisomeric structure of HMF several companies have developed HMF substitutes (HMFS). HMFS are made by enzyme-catalyzed acidolysis (or alcoholysis and esterification) using tripalmitin, unsaturated free fatty acids (mainly C18:1) together with an immobilized recombinant sn-1/3-regioselective lipase. The price of HMFS is substantially higher than that of conventional vegetable fat blends, primarily reflecting the added cost of enzyme-based catalysis, including generation of organic solvent waste. Different grades of HMFS are also available, providing a complete fat phase with between ˜40 and ˜70% of C16:0 at the sn-2 position. True HMF mimetics (with >70% of C16:0 at sn-2) are most expensive to produce because they require a two-step catalytic process and a pure tripalmitin feedstock derived from palm oil by special fractionation procedures and chemical randomisation. The tension between price and quality is one factor that has likely restricted the use of HMFS and despite mounting clinical evidence that this ingredient is beneficial, it is currently only found in around 10% of infant formula, particularly premium products formulated and marketed for ease-of-digestion. Even in these products, there remains a substantial gap in C16:0 enrichment at the sn-2 position versus HMF.

It is, therefore, an object of the present invention to seek to alleviate the above identified problems.

### SUMMARY OF THE INVENTION

According to one aspect of the present invention, there is provided a non-human organism for producing triacylglycerol wherein the non-human organism is genetically modified to express a lysophosphatidic acid acyltransferase specific for C16:0-Coenzyme A (C16:0 LPAT) and wherein, once expressed, the C16:0 LPAT is localised in the endoplasmic reticulum.

Preferably, activity of native endoplasmic reticulum lysophosphatidic acid acyltransferase (ER LPAT) is suppressed or prevented in the organism.

According to another aspect of the present invention, there is provided a non-human organism for producing triacylglycerol in which:
(a) a lysophosphatidic acid acyltransferase specific for C16:0-Coenzyme A (C16:0 LPAT) is localised in the endoplasmic reticulum; and
(b) activity of native endoplasmic reticulum lysophosphatidic acid acyltransferase (ER LPAT) is suppressed or prevented.

Preferably, the activity of diacylglycerol conversion to and from phosphatidylcholine is suppressed or prevented.

Preferably, the activity of one or more native endoplasmic reticulum lysophosphatidic acid acyltransferases (ER LPATs) is suppressed or prevented.

According to another aspect of the present invention, there is provided a non-human organism for producing triacylglycerol in which:
(a) a lysophosphatidic acid acyltransferase specific for C16:0-Coenzyme A (C16:0 LPAT) is localised in the endoplasmic reticulum;
(b) activity of native endoplasmic reticulum lysophosphatidic acid acyltransferase (ER LPAT) is suppressed or prevented; and
(c) activity of diacylglycerol conversion to and from phosphatidylcholine is suppressed or prevented.

Remarkably, as discussed in further detail herein, by providing a lysophosphatidic acid acyltransferase specific for C16:0-Coenzyme A (CoA) which is localised in the endoplasmic reticulum, the enzyme operates within the cytosolic glycerolipid biosynthetic pathway to esterify C16:0 to the middle position during TAG biosynthesis. This, combined with suppression of native endoplasmic reticulum lysophosphatidic acid acyltransferase and optionally suppression of diacylglycerol conversion to and from phosphatidylcholine, means that a much greater amount of C16:0 is esterified to the middle position on the glycerol backbone than to the outer positions.

Preferably, the organism comprises an endoplasmic reticulum.

Preferably, the organism is a plant, fungi, yeast or algae.

Preferably, the organism is not a mammal.

Preferably, the organism is a plant. Preferably, the plant is an oilseed plant.

Preferably, the organism is a plant belonging to the order of Brassicales, Asterales, Fabales, Malpighiales, Malvales, Rosales, Lamiales, Solanales, Arecales or Poales. Most preferably, the plant belongs to the order Brassicales or Asterales, more preferably Brassicales.

Preferably, the organism is a plant belonging to the family of Brassicaceae, Asteraceae, Fabaceae, Linaceae, Malvaceae, Cannabaceae, Pedaliaceae, Oleaceae, Solanaceae, Arecaceae or Poaceae. Most preferably, the plant belongs to the family Brassicaceae or Asteraceae, more preferably Brassicaceae.

Preferably, the organism is a plant belonging to the genus of Arabidopsis, Camelina, Brassica, Thlaspi, Lepidium, Helianthus, Glycine, Arachis, Linum, Nicotiana, Gossypium, Cannabis, Sesamum, Olea, Elaeis, Zea, Avena or Oryza. Most preferably, the plant belongs to the genus Arabidopsis, Camelina, Brassica or Helianthus.

Preferably, the plant is selected from Arabidopsis thaliana, Camelina sativa, Brassica napus, Brassica carinata, Brassica oleracea, Brassica rapa, Brassica juncea, Thlaspi arvense, Lepidium sativum, Helianthus annuus, Glycine max, Arachis hypogaea, Linum usitatissimum, Gossypium hirsutum, Cannabis sativa, Sesamum indicum, Olea europaea, Nicotiana benthamiana, Elaeis guineensis, Elaeis oleifera, Zea mays, Avena sativa or Oryza sativa.

Preferably, the plant is selected from Arabidopsis thaliana, Helianthus annuus, Glycine max, Camelina sativa and Brassica napus.

Preferably, the plant is selected from Arabidopsis thaliana, Camelina sativa and Brassica napus.

Preferably, the plant is Arabidopsis thaliana.

Preferably, the organism is a plant from which triacylglycerol can be extracted, for example from the seeds, fruits or leaves of the plant.

Preferably, an oilseed plant is a plant from which triacylglycerol can be extracted from seeds or fruits.

Preferably, the organism is a yeast.

Preferably, the organism is a fermentative and/or respiratory yeast.

Preferably, the organism is a yeast of an oleaginous species.

Preferably, the organism is a yeast capable of accumulating at least about 20% triacylglycerol in cellular biomass.

Preferably, the organism is a fungus belonging to the order of Saccharomycesles, Saccharomyces Tremellales, Ustilaginales, Sporidiobolales, Mucorales, Mortierellales or Eurotiales. Most preferably, the organism is from the order of Saccharomycesles.

Preferably, the organism is a fungus belonging to the family of Dipodascaceae, Saccharomycesceae, Saccharomycopsidaceae, Tremellaceae, Ustilaginaceae, Sporidiobolaceae, Trichosporonaceae, Phaffomycetaceae, Trichomonascaceae, Mucoraceae, Mortierellaceae, Cunninghamellaceae or Trichocomaceae. Most preferably, the organism is from the family of Dipodascaceae.

Preferably, the organism is a fungus belonging to the genus of Saccharomyces, Yarrowia, Cryptococcus, Candida, Rhodosporidium, Rhodotorula, Lipomyces, Trichosporon, Wickerhamomyces, Pichia, Endomycopsis, Zygoascus, Mucor, Mortierella, Cunninghamella or Aspergillus. Most preferably, the organism is from the genus of Yarrowia.

Preferably, the organism is an obligate respiratory and crabtree-negative yeast.

Preferably, the organism is Yarrowia lipolytica.

Preferably, the organism is Yarrowia lipolytica and the C16:0 LPAT is CreLPAT.

Preferably, the organism is a gsy 14-CreLPAT strain of Yarrowia lipolytica.

Preferably, the organism is cultured in a media comprising a carbon source, wherein the carbon source comprises one or more sugars.

Preferably, the one or more sugars comprise one or more fermentable sugars.

Preferably, the one or more sugars is selected from one or more of xylose, lactose, cellulose, glucose, fructose, sucrose, or hydrolysed lignocellulosic materials.

Preferably, the organism is cultured in a media comprising a carbon source, wherein the carbon source comprises one or more fatty acids and/or fatty acid esters.

Preferably, the carbon source comprises C16:0.

Preferably, the carbon source comprises a mixture of fatty acids and/or fatty acid esters wherein at least about 30% w/w of the fatty acids and/or fatty acid esters comprises C16:0 and/or at least about 30% w/w of the fatty acids and/or fatty acid esters comprises C18:1.

Preferably, the organism is cultured in a media comprising a carbon source, wherein the carbon source comprises one or more vegetable oils.

Preferably, the organism is cultured in a media comprising a carbon source, wherein the carbon source comprises a mixture of (i) one or more sugars and (ii) one or more fatty acids and/or fatty acid esters.

Preferably, the organism is cultured in a media comprising a carbon source, wherein the carbon source comprises a mixture of (i) one or more sugars and (ii) one or more oils, preferably vegetable oils.

Preferably, the carbon source comprises glycerol.

Preferably, the organism is cultured in a media comprising a carbon source, wherein the carbon source is selected from glycerol, glucose and/or palm oil.

Preferably, the carbon source comprises palm oil.

Preferably, the carbon source comprises palm oil in combination with glucose and/or glycerol.

Preferably, the carbon source is present at a concentration of at least about 5 gL−1, preferably at least about 10 gL−1, preferably at least about 15 gL−1, preferably at least about 20 gL−1.

Preferably, the carbon source is present at a concentration of between about 5 gL−1 and about 35 gL−1, preferably between about 10 gL−1 and about 30 gL−1, preferably between about 15 gL−1 and about 25 gL−1, preferably at a concentration of about 20 gL−1.

Preferably, the media comprises a first carbon source at a concentration of at least about 2.5 gL−1 and a second carbon source at a concentration of at least about 2.5 gL−1.

Preferably, the media comprises a first carbon source at a concentration of at least about 5 gL−1, preferably at least about 10 gL−1, and a second carbon source at a concentration of at least about 5 gL−1, preferably at least about 10 gL−1.

Preferably, the media comprises a first carbon source at a concentration of between about 2.5 gL−1 and about 20 gL−1, and a second carbon source at a concentration of between about 2.5 gL−1 and about 20 gL−1.

Preferably, the media comprises a first carbon source at a concentration of between about 5 gL−1 and about 15 gL−1, preferably about 10 gL−1, and a second carbon source at a concentration of between about 5 gL−1 and about 15 gL−1, preferably about 10 gL−1.

Preferably, the first carbon source comprises one or more sugars.

Preferably, the one or more sugars comprise one or more fermentable sugars.

Preferably, the one or more sugars is selected from one or more of xylose, lactose, cellulose, glucose, fructose, sucrose, or hydrolysed lignocellulosic materials.

Preferably, the first carbon source glycerol and/or one or more sugars.

Preferably, the second carbon source comprises one or more fatty acids and/or fatty acid esters.

Preferably, the second carbon source comprises C16:0.

Preferably, the second carbon source comprises a mixture of fatty acids and/or fatty acid esters wherein at least about 30% w/w of the fatty acids and/or fatty acid esters comprises C16:0 and/or at least about 30% w/w of the fatty acids and/or fatty acid esters comprises C18:1.

Preferably, the second carbon source comprises one or more vegetable oils.

Preferably, the second carbon source comprises palm oil.

Preferably, the media is a nitrogen limited media.

Preferably, the media comprises carbon and nitrogen, wherein the carbon to nitrogen ratio is greater than about 40.

Preferably, the organism is a yeast and the C16:0 LPAT is AGPAT1.

Preferably, the organism is Saccharomyces cerevisiae and the C16:0 LPAT is AGPAT1.

According to another aspect of the present invention, there is provided a non-human organism, wherein the organism is Saccharomyces cerevisiae genetically modified to express AGPAT1.

Preferably, the organism is a Prototheca algae.

Preferably, the C16:0 LPAT is selected from:
(i) a chloroplast LPAT which lacks a functional chloroplast targeting sequence, preferably a plant chloroplast LPAT which lacks a functional chloroplast targeting sequence;
(ii) AGPAT1 (Human 1-acylglycerol-3-phosphate O-acyltransferase isoform 1);
(iii) CreLPAT;
(iv) Nannochloropsis sp. LPAT2;
(v) Nannochloropsis sp. LPAT3;
(vi) Nannochloropsis sp. LPAT4; and
(vii) Synechocystis sp. LPAT.

Preferably, the organism is treated or genetically modified to disrupt the chloroplast targeting sequence.

Preferably, the chloroplast LPAT is treated or genetically modified to disrupt the chloroplast targeting sequence.

Preferably, the chloroplast LPAT is a modified chloroplast LPAT which lacks at least a part of, preferably all of, a chloroplast targeting sequence, preferably a modified plant chloroplast LPAT which lacks at least a part of, preferably all of, a chloroplast targeting sequence.

Preferably, the chloroplast targeting sequence is an N-terminal chloroplast targeting sequence.

Preferably, the organism is modified or treated to disrupt the chloroplast LPAT chloroplast targeting sequence, for example by genetic modification, preferably by genetic modification of the chloroplast targeting sequence.

Preferably, said genetic modification comprises the addition or removal of nucleic acid and/or amino acid residues.

Preferably, the organism is genetically modified to express a lysophosphatidic acid acyltransferase specific for C16:0-CoA (C16:0 LPAT) localised in the endoplasmic reticulum.

Preferably, the organism is a plant and the C16:0 LPAT is expressed under the control of a seed-specific promoter, preferably the oleosin, napin or glycinin seed specific promoter, most preferably, the seed-specific soybean glycinin-1 promoter (ProGLY).

Preferably, the organism is treated or genetically modified to suppress or prevent activity of native ER LPAT.

Preferably, the organism is treated or genetically modified to suppress or prevent activity of one or more native ER LPATs.

Preferably, the ER LPAT is not specific for C16:0-CoA.

Preferably, the activity of native ER LPAT is suppressed or prevented using artificial micro-RNA.

Preferably, the activity of native ER LPAT is suppressed or prevented using RNAi and/or genome editing and/or mutation breeding.

Preferably, the artificial micro-RNA is under the control of a seed-specific promoter, preferably the oleosin, napin or glycinin seed specific promoter.

Preferably, the organism comprises a disruptive insertion in a non-coding region 5′ of the ER LPAT translational start site. Preferably, the disruptive insertion is at about 139 bp 5′ of the ER LPAT translational start site. Preferably, the disruptive insertion is 139 bp 5′ of the ER LPAT translational start site. Preferably, the disruptive insertion is a T-DNA insertion.

Alternatively, the disruptive insertion is at about 302 bp 5′, for example 302 bp 5′, of the ER LPAT translational start site.

Preferably, conversion of diacylglycerol to phosphatidylcholine is suppressed or prevented in the organism.

Preferably, conversion of diacylglycerol to phosphatidylcholine is suppressed or prevented in the organism by suppressing or preventing the activity of choline phosphotransferase (CPT1) and/or ethanolamine phosphotransferase (EPT1).

Preferably, the organism is treated or genetically modified to disrupt CPT1 and/or EPT1.

Preferably, the activity of phosphatidylcholine:diacylglycerol cholinephosphotransferase (PDCT) is suppressed or prevented in the organism.

Preferably, the activity of one or more phosphatidylcholine:diacylglycerol cholinephosphotransferases (PDCTs) is suppressed or prevented in the organism.

Preferably, diacylglycerol conversion to and/or from phosphatidylcholine is reduced by suppressing or preventing the activity of phosphatidylcholine:diacylglycerol cholinephosphotransferase (PDCT) in the organism, for example by suppressing or preventing the activity of one or more phosphatidylcholine:diacylglycerol cholinephosphotransferases (PDCTs) in the organism.

Preferably, the organism is treated or genetically modified to increase total C16:0 content.

Preferably, the organism is a plant which is treated or genetically modified to increase total C16:0 content, preferably in the seeds, fruits or the leaves, most preferably in the seeds or fruits.

Preferably, the organism expresses a FATB thioesterase, preferably an Arabidopsis thaliana FATB thioesterase.

Preferably, the organism is genetically modified to expresses the/a FATB thioesterase.

Preferably, the organism is treated or genetically modified to disrupt plastidic beta-ketoacyl-ACP synthase II.

Preferably, the organism is treated or genetically modified to disrupt the KASII gene FAB1 (At1g74960).

Preferably, the organism is treated or genetically modified to disrupt the fatty acid elongase gene (FAE1).

Preferably, the organism is treated or genetically modified to disrupt the fatty acid desaturase 2 gene (FAD2).

Preferably, the organism expresses a FATB thioesterase, preferably an Arabidopsis thaliana FATB thioesterase and is treated or genetically modified to disrupt the fatty acid desaturase 2 gene (FAD2).

Preferably, the organism is treated or genetically modified to express the/a FATB thioesterase gene and is treated or genetically modified to disrupt the fatty acid desaturase 2 gene (FAD2).

Preferably, the organism, preferably a plant, is treated or genetically modified to disrupt the KASII gene FAB1, fatty acid elongase gene (FAE1) and fatty acid desaturase 2 gene (FAD2).

Preferably, (i) the organism, preferably a plant, is treated or genetically modified to disrupt the KASII gene FAB1, fatty acid elongase gene (FAE1), and fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

Preferably, (i) the organism, preferably a plant, is treated or genetically modified to disrupt the KASII gene FAB1, fatty acid elongase gene (FAE1), and fatty acid desaturase 2 gene (FAD2), (ii) conversion of diacylglycerol to phosphatidylcholine is suppressed or prevented in the organism, (iii) activity of native endoplasmic reticulum lysophosphatidic acid acyltransferase (ER LPAT) is suppressed or prevented in the organism, and (iv) the C16:0 LPAT is ΔCTS-LPAT1 or AGPAT1.

Preferably, (i) the organism, preferably a plant, expresses a FATB thioesterase, preferably an Arabidopsis thaliana FATB thioesterase and is treated or genetically modified to disrupt the fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

Preferably, (i) the organism, preferably a plant, expresses a FATB thioesterase, preferably an Arabidopsis thaliana FATB thioesterase and is treated or genetically modified to disrupt the fatty acid desaturase 2 gene (FAD2), (ii) conversion of diacylglycerol to phosphatidylcholine is suppressed or prevented in the organism, (iii) activity of native endoplasmic reticulum lysophosphatidic acid acyltransferase (ER LPAT) is suppressed or prevented in the organism, and (iv) the C16:0 LPAT is ΔCTS-LPAT1 or AGPAT1.

According to another aspect of the present invention, there is provided a non-human organism, preferably a plant, in which a C16:0 LPAT is expressed and wherein (i) the organism is treated or genetically modified to disrupt the KASII gene FAB1, fatty acid elongase gene (FAE1), and fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

According to another aspect of the present invention, there is provided a non-human organism, preferably a plant, in which a C16:0 LPAT is expressed and wherein (i) the organism expresses a FATB thioesterase, preferably an Arabidopsis thaliana FATB thioesterase and is treated or genetically modified to disrupt the fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

According to another aspect of the present invention, there is provided a non-human organism, preferably a plant, in which ΔCTS-LPAT1 is expressed and wherein (i) the organism is treated or genetically modified to disrupt the KASII gene FAB1, fatty acid elongase gene (FAE1), and fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

According to another aspect of the present invention, there is provided a non-human organism, preferably a plant, in which ΔCTS-LPAT1 is expressed and wherein (i) the organism expresses a FATB thioesterase, preferably an Arabidopsis thaliana FATB thioesterase and is treated or genetically modified to disrupt the fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

According to another aspect of the present invention, there is provided a non-human organism, preferably a plant, in which AGPAT1 is expressed and wherein (i) the organism is treated or genetically modified to disrupt the KASII gene FAB1, fatty acid elongase gene (FAE1), and fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

According to another aspect of the present invention, there is provided a non-human organism, preferably a plant, in which AGPAT1 is expressed and wherein (i) the organism expresses a FATB thioesterase, preferably an Arabidopsis thaliana FATB thioesterase and is treated or genetically modified to disrupt the fatty acid desaturase 2 gene (FAD2), (ii) conversion of diacylglycerol to phosphatidylcholine is suppressed or prevented in the organism, and (iii) activity of native endoplasmic reticulum lysophosphatidic acid acyltransferase (ER LPAT) is suppressed or prevented in the organism.

Preferably, conversion of diacylglycerol to phosphatidylcholine is suppressed or prevented in the organism by suppressing or preventing the activity of phosphatidylcholine:diacylglycerol cholinephosphotransferase (PDCT).

Preferably, conversion of diacylglycerol to phosphatidylcholine is suppressed or prevented in the organism by suppressing or preventing the activity of choline phosphotransferase (CPT1) and/or ethanolamine phosphotransferase (EPT1).

Preferably, the organism is treated or genetically modified to expresses the/a FATB thioesterase gene.

According to another aspect of the present invention, there is provided a non-human organism, preferably a plant, in which a chloroplast lysophosphatidic acid acyltransferase (LPAT) is expressed, wherein said chloroplast LPAT lacks a functional chloroplast targeting signal.

As detailed above, remarkably, when the N-terminal chloroplast targeting signal is prevented from functioning, it has been found that the chloroplast LPAT relocates to the endoplasmic reticulum where it esterifies C16:0 to the sn-2 position on the glycerol backbone.

Preferably, the organism is treated or genetically modified to disrupt the chloroplast targeting sequence.

Preferably, the chloroplast LPAT is treated or genetically modified to disrupt the chloroplast targeting sequence.

Preferably, the chloroplast LPAT is a modified chloroplast LPAT which lacks at least a part of, preferably all of, a chloroplast targeting sequence, preferably a modified plant chloroplast LPAT which lacks at least a part of, preferably all of, a chloroplast targeting sequence.

Preferably, the chloroplast targeting sequence is an N-terminal chloroplast targeting sequence.

Preferably, the organism is modified or treated to disrupt the chloroplast LPAT chloroplast targeting sequence, for example by genetic modification, preferably by genetic modification of the chloroplast targeting sequence.

Preferably, said genetic modification comprises the addition or removal of nucleic acid and/or amino acid residues.

Preferably, activity of native endoplasmic reticulum (ER) LPAT is suppressed or prevented in the organism.

Preferably, activity of one or more native endoplasmic reticulum (ER) LPATs is suppressed or prevented in the organism.

Preferably, the non-human organism is polyploid.

Within this specification, reference to “suppressed” means reduced but not prevented.

Preferably, suppressed means reduced by at least about 50%, preferably by at least about 60%, preferably by at least about 70%, preferably by at least about 80%, preferably by at least about 85%, preferably by at least about 90%, preferably by at least about 95%, preferably by at least about 98%.

Preferably, suppressed means reduced by about 83%.

Preferably, suppressed means reduced by between about 50% and about 98%, preferably between about 60% and about 95%, preferably between about 70% and about 90%, preferably between about 80% and about 90%.

Preferably, the C16:0 LPAT is codon optimised for expression in the organism.

Preferably, the C16:0 LPAT is codon optimised for expression in plants, fungi, yeast or algae. Most preferably, the lysophosphatidic acid acyltransferase is codon optimised for expression in plants or yeast.

Preferably, reference to a “modified chloroplast lysophosphatidic acid acyltransferase (LPAT)” means a protein encoded by a nucleotide sequence having at least about 80% sequence identity to SEQ ID NO: 1 or SEQ ID NO:38, preferably SEQ ID NO:1, preferably at least about 85% sequence identity, preferably at least about 90% sequence identity, preferably at least about 95% sequence identity, preferably at least about 98% sequence identity, preferably at least about 99% sequence identity to SEQ ID NO: 1 or SEQ ID NO:38, preferably to SEQ ID NO:1.

Preferably, reference to a “modified chloroplast lysophosphatidic acid acyltransferase (LPAT)” means a protein comprising an amino acid sequence having at least about 80% sequence identity to SEQ ID NO: 2, preferably at least about 85% sequence identity, preferably at least about 90% sequence identity, preferably at least about 95% sequence identity, preferably at least about 98% sequence identity, preferably at least about 99% sequence identity to SEQ ID NO:2.

Preferably, reference to a “modified chloroplast lysophosphatidic acid acyltransferase (LPAT)” means a protein encoded by the nucleotide sequence of SEQ ID NO:1 or SEQ ID NO:38 and/or a protein comprising the amino acid sequence of SEQ ID NO:2.

Preferably, the modified chloroplast LPAT is expressed under the control of a seed-specific promoter.

Preferably, the modified chloroplast LPAT is expressed under the control of the seed-specific soybean glycinin-1 promoter (ProGLY).

Preferably, the modified chloroplast LPAT is a truncated chloroplast LPAT which lacks a functional chloroplast targeting signal.

Preferably, the organism comprises a disruptive insertion in a non-coding region 5′ of the ER LPAT translational start site. Preferably, the disruptive insertion is at about 139 bp 5′ of the ER LPAT translational start site. Preferably, the disruptive insertion is 139 bp 5′ of the ER LPAT translational start site. Preferably, the disruptive insertion is a T-DNA insertion.

Preferably, the activity of phosphatidylcholine:diacylglycerol cholinephosphotransferase (PDCT) is suppressed or prevented in the organism.

Preferably, reference to “AGPAT1” means a protein comprising an amino acid sequence having at least about 80% sequence identity to SEQ ID NO:40, preferably, at least about 85% sequence identity, preferably at least about 90% sequence identity, preferably at least about 95% sequence identity, preferably at least about 98% sequence identity, preferably at least about 99% sequence identity to SEQ ID NO: 40.

Preferably, reference to “AGPAT1” means a protein encoded by a nucleotide sequence having at least about 80% sequence identity to SEQ ID NO:26, 37 or 39, preferably, at least about 85% sequence identity, preferably at least about 90% sequence identity, preferably at least about 95% sequence identity, preferably at least about 98% sequence identity, preferably at least about 99% sequence identity to SEQ ID NO:26, 37 or 39.

Preferably, reference to a “AGPAT1” means a protein encoded by the nucleotide sequence of SEQ ID NO:26, 37 or 39 and/or a protein comprising the amino acid sequence of SEQ ID NO:40.

Preferably, reference to “CreLPAT” means a protein comprising an amino acid sequence having at least about 80% sequence identity to SEQ ID NO:42, preferably, at least about 85% sequence identity, preferably at least about 90% sequence identity, preferably at least about 95% sequence identity, preferably at least about 98% sequence identity, preferably at least about 99% sequence identity to SEQ ID NO: 42.

Preferably, reference to “CreLPAT” means a protein encoded by a nucleotide sequence having at least about 80% sequence identity to SEQ ID NO:36 or SEQ ID NO:41, preferably, at least about 85% sequence identity, preferably at least about 90% sequence identity, preferably at least about 95% sequence identity, preferably at least about 98% sequence identity, preferably at least about 99% sequence identity to SEQ ID NO:36 or SEQ ID NO:41.

Preferably, reference to a “CreLPAT” means a protein encoded by the nucleotide sequence of SEQ ID NO:36 or SEQ ID NO:41 and/or a protein comprising the amino acid sequence of SEQ ID NO: 42.

Preferably, reference to “Nannochloropsis sp. LPAT2” means a protein comprising an amino acid sequence having at least about 80% sequence identity to SEQ ID NO:44, preferably, at least about 85% sequence identity, preferably at least about 90% sequence identity, preferably at least about 95% sequence identity, preferably at least about 98% sequence identity, preferably at least about 99% sequence identity to SEQ ID NO: 44.

Preferably, reference to “Nannochloropsis sp. LPAT2” means a protein encoded by a nucleotide sequence having at least about 80% sequence identity to SEQ ID NO:43, preferably, at least about 85% sequence identity, preferably at least about 90% sequence identity, preferably at least about 95% sequence identity, preferably at least about 98% sequence identity, preferably at least about 99% sequence identity to SEQ ID NO:43.

Preferably, reference to a “Nannochloropsis sp. LPAT2” means a protein encoded by the nucleotide sequence of SEQ ID NO:43 and/or a protein comprising the amino acid sequence of SEQ ID NO: 44.

Preferably, reference to “Nannochloropsis sp. LPAT3” means a protein comprising an amino acid sequence having at least about 80% sequence identity to SEQ ID NO:46, preferably, at least about 85% sequence identity, preferably at least about 90% sequence identity, preferably at least about 95% sequence identity, preferably at least about 98% sequence identity, preferably at least about 99% sequence identity to SEQ ID NO: 46.

Preferably, reference to “Nannochloropsis sp. LPAT3” means a protein encoded by a nucleotide sequence having at least about 80% sequence identity to SEQ ID NO:45, preferably, at least about 85% sequence identity, preferably at least about 90% sequence identity, preferably at least about 95% sequence identity, preferably at least about 98% sequence identity, preferably at least about 99% sequence identity to SEQ ID NO:45.

Preferably, reference to a “Nannochloropsis sp. LPAT3” means a protein encoded by the nucleotide sequence of SEQ ID NO:45 and/or a protein comprising the amino acid sequence of SEQ ID NO: 46.

Preferably, reference to “Nannochloropsis sp. LPAT4” means a protein comprising an amino acid sequence having at least about 80% sequence identity to SEQ ID NO:48, preferably, at least about 85% sequence identity, preferably at least about 90% sequence identity, preferably at least about 95% sequence identity, preferably at least about 98% sequence identity, preferably at least about 99% sequence identity to SEQ ID NO: 48.

Preferably, reference to “Nannochloropsis sp. LPAT4” means a protein encoded by a nucleotide sequence having at least about 80% sequence identity to SEQ ID NO:47, preferably, at least about 85% sequence identity, preferably at least about 90% sequence identity, preferably at least about 95% sequence identity, preferably at least about 98% sequence identity, preferably at least about 99% sequence identity to SEQ ID NO:47.

Preferably, reference to a “Nannochloropsis sp. LPAT4” means a protein encoded by the nucleotide sequence of SEQ ID NO:47 and/or a protein comprising the amino acid sequence of SEQ ID NO: 48.

Preferably, reference to a “Synechocystis sp. LPAT” means a protein comprising an amino acid sequence having at least about 80% sequence identity to SEQ ID NO:50, preferably, at least about 85% sequence identity, preferably at least about 90% sequence identity, preferably at least about 95% sequence identity, preferably at least about 98% sequence identity, preferably at least about 99% sequence identity to SEQ ID NO:50.

Preferably, reference to a “Synechocystis sp. LPAT” means a protein encoded by a nucleotide sequence having at least about 80% sequence identity to SEQ ID NO:49, preferably, at least about 85% sequence identity, preferably at least about 90% sequence identity, preferably at least about 95% sequence identity, preferably at least about 98% sequence identity, preferably at least about 99% sequence identity to SEQ ID NO:49.

Preferably, reference to a “Synechocystis sp. LPAT” means a protein encoded by the nucleotide sequence of SEQ ID NO:49 and/or a protein comprising the amino acid sequence of SEQ ID NO:50.

Preferably, reference to “native endoplasmic reticulum lysophosphatidic acid acyltransferase” means a protein comprising an amino acid sequence having at least about 80% sequence identity to SEQ ID NO: 52, SEQ ID NO:54, SEQ ID NO:56, SEQ ID NO:58, SEQ ID NO:60 or SEQ ID NO:62, preferably, at least about 85% sequence identity, preferably at least about 90% sequence identity, preferably at least about 95% sequence identity, preferably at least about 98% sequence identity, preferably at least about 99% sequence identity to SEQ ID NO:52, SEQ ID NO:54, SEQ ID NO:56, SEQ ID NO:58, SEQ ID NO:60 or SEQ ID NO:62.

Preferably, reference to “native endoplasmic reticulum lysophosphatidic acid acyltransferase” means a protein encoded by a nucleotide sequence having at least about 80% sequence identity to SEQ ID NO: 51, SEQ ID NO:53, SEQ ID NO:55, SEQ ID NO:57, SEQ ID NO:59 or SEQ ID NO:61, preferably, at least about 85% sequence identity, preferably at least about 90% sequence identity, preferably at least about 95% sequence identity, preferably at least about 98% sequence identity, preferably at least about 99% sequence identity to SEQ ID NO:51, SEQ ID NO:53, SEQ ID NO:55, SEQ ID NO:57, SEQ ID NO:59 or SEQ ID NO:61.

Preferably, reference to a “native endoplasmic reticulum lysophosphatidic acid acyltransferase” means a protein encoded by the nucleotide sequence of SEQ ID NO:51, SEQ ID NO:53, SEQ ID NO:55, SEQ ID NO:57, SEQ ID NO:59 or SEQ ID NO:61 and/or a protein comprising the amino acid sequence of SEQ ID NO:52, SEQ ID NO:54, SEQ ID NO:56, SEQ ID NO:58, SEQ ID NO: 60 or SEQ ID NO:62.

Preferably, reference to “phosphatidylcholine:diacylglycerol cholinephosphotransferase” means a protein comprising an amino acid sequence having at least about 80% sequence identity to SEQ ID NO: 64, SEQ ID NO:66, SEQ ID NO: 68, SEQ ID NO: 70 or SEQ ID NO: 72, preferably, at least about 85% sequence identity, preferably at least about 90% sequence identity, preferably at least about 95% sequence identity, preferably at least about 98% sequence identity, preferably at least about 99% sequence identity to SEQ ID NO:64, SEQ ID NO:66, SEQ ID NO: 68, SEQ ID NO: 70 or SEQ ID NO:72.

Preferably, reference to “phosphatidylcholine:diacylglycerol cholinephosphotransferase” means a protein encoded by a nucleotide sequence having at least about 80% sequence identity to SEQ ID NO: 63, SEQ ID NO: 65, SEQ ID NO: 67, SEQ ID NO: 69 or SEQ ID NO:71, preferably, at least about 85% sequence identity, preferably at least about 90% sequence identity, preferably at least about 95% sequence identity, preferably at least about 98% sequence identity, preferably at least about 99% sequence identity to SEQ ID NO: 63, SEQ ID NO: 65, SEQ ID NO: 67, SEQ ID NO: 69 or SEQ ID NO:71.

Preferably, reference to a “phosphatidylcholine:diacylglycerol cholinephosphotransferase” means a protein encoded by the nucleotide sequence of SEQ ID NO:63, SEQ ID NO:65, SEQ ID NO:67, SEQ ID NO:69 or SEQ ID NO:71 and/or a protein comprising the amino acid sequence of SEQ ID NO: 64, SEQ ID NO: 66, SEQ ID NO:68, SEQ ID NO: 70 or SEQ ID NO:72.

Preferably, reference to “Arabidopsis thaliana FATB thioesterase” means a protein comprising an amino acid sequence having at least about 80% sequence identity to SEQ ID NO:73, preferably, at least about 85% sequence identity, preferably at least about 90% sequence identity, preferably at least about 95% sequence identity, preferably at least about 98% sequence identity, preferably at least about 99% sequence identity to SEQ ID NO: 73.

Preferably, reference to “Arabidopsis thaliana FATB thioesterase” means a protein encoded by a nucleotide sequence having at least about 80% sequence identity to SEQ ID NO:33, preferably, at least about 85% sequence identity, preferably at least about 90% sequence identity, preferably at least about 95% sequence identity, preferably at least about 98% sequence identity, preferably at least about 99% sequence identity to SEQ ID NO:33.

Preferably, reference to a “Arabidopsis thaliana FATB thioesterase” means a protein encoded by the nucleotide sequence of SEQ ID NO:33 and/or a protein comprising the amino acid sequence of SEQ ID NO:73.

Preferably, reference to “FAB1” means a protein comprising an amino acid sequence having at least about 80% sequence identity to SEQ ID NO:79, preferably, at least about 85% sequence identity, preferably at least about 90% sequence identity, preferably at least about 95% sequence identity, preferably at least about 98% sequence identity, preferably at least about 99% sequence identity to SEQ ID NO: 79.

Preferably, reference to “FAB1” means a protein encoded by a nucleotide sequence having at least about 80% sequence identity to SEQ ID NO:78, preferably, at least about 85% sequence identity, preferably at least about 90% sequence identity, preferably at least about 95% sequence identity, preferably at least about 98% sequence identity, preferably at least about 99% sequence identity to SEQ ID NO: 78.

Preferably, reference to “FAB1” means a protein encoded by the nucleotide sequence of SEQ ID NO: 78 and/or a protein comprising the amino acid sequence of SEQ ID NO:79.

Preferably, reference to “FAD2” means a protein comprising an amino acid sequence having at least about 80% sequence identity to SEQ ID NO:81 or 85, preferably, at least about 85% sequence identity, preferably at least about 90% sequence identity, preferably at least about 95% sequence identity, preferably at least about 98% sequence identity, preferably at least about 99% sequence identity to SEQ ID NO:81 or 85.

Preferably, reference to “FAD2” means a protein encoded by a nucleotide sequence having at least about 80% sequence identity to SEQ ID NO:80 or 84, preferably, at least about 85% sequence identity, preferably at least about 90% sequence identity, preferably at least about 95% sequence identity, preferably at least about 98% sequence identity, preferably at least about 99% sequence identity to SEQ ID NO:80 or 84.

Preferably, reference to “FAD2” means a protein encoded by the nucleotide sequence of SEQ ID NO: 80 or 84 and/or a protein comprising the amino acid sequence of SEQ ID NO:81 or 85.

Preferably, reference to “FAE1” means a protein comprising an amino acid sequence having at least about 80% sequence identity to SEQ ID NO:83 or 87, preferably, at least about 85% sequence identity, preferably at least about 90% sequence identity, preferably at least about 95% sequence identity, preferably at least about 98% sequence identity, preferably at least about 99% sequence identity to SEQ ID NO:83 or 87.

Preferably, reference to “FAE1” means a protein encoded by a nucleotide sequence having at least about 80% sequence identity to SEQ ID NO:82 or 86, preferably, at least about 85% sequence identity, preferably at least about 90% sequence identity, preferably at least about 95% sequence identity, preferably at least about 98% sequence identity, preferably at least about 99% sequence identity to SEQ ID NO:82 or 86.

Preferably, reference to a “FAE1” means a protein encoded by the nucleotide sequence of SEQ ID NO: 83 or 87 and/or a protein comprising the amino acid sequence of SEQ ID NO:82 or 86.

Preferably, reference to “choline phosphotransferase (CPT1)” means a protein comprising an amino acid sequence having at least about 80% sequence identity to SEQ ID NO:89, preferably, at least about 85% sequence identity, preferably at least about 90% sequence identity, preferably at least about 95% sequence identity, preferably at least about 98% sequence identity, preferably at least about 99% sequence identity to SEQ ID NO:89.

Preferably, reference to “choline phosphotransferase (CPT1)” means a protein encoded by a nucleotide sequence having at least about 80% sequence identity to SEQ ID NO:88, preferably, at least about 85% sequence identity, preferably at least about 90% sequence identity, preferably at least about 95% sequence identity, preferably at least about 98% sequence identity, preferably at least about 99% sequence identity to SEQ ID NO:88.

Preferably, reference to a “choline phosphotransferase (CPT1)” means a protein encoded by the nucleotide sequence of SEQ ID NO:88 and/or a protein comprising the amino acid sequence of SEQ ID NO: 89.

Preferably, reference to “ethanolamine phosphotransferase (EPT1)” means a protein comprising an amino acid sequence having at least about 80% sequence identity to SEQ ID NO:91, preferably, at least about 85% sequence identity, preferably at least about 90% sequence identity, preferably at least about 95% sequence identity, preferably at least about 98% sequence identity, preferably at least about 99% sequence identity to SEQ ID NO:91.

Preferably, reference to “ethanolamine phosphotransferase (EPT1)” means a protein encoded by a nucleotide sequence having at least about 80% sequence identity to SEQ ID NO:90, preferably, at least about 85% sequence identity, preferably at least about 90% sequence identity, preferably at least about 95% sequence identity, preferably at least about 98% sequence identity, preferably at least about 99% sequence identity to SEQ ID NO:90.

Preferably, reference to a “ethanolamine phosphotransferase (EPT1)” means a protein encoded by the nucleotide sequence of SEQ ID NO:90 and/or a protein comprising the amino acid sequence of SEQ ID NO:91.

According to another aspect of the present invention, there is provided a cell of a non-human organism described herein. Preferably, the cell is a recombinant cell.

According to another aspect of the present invention, there is provided a seed for producing a plant as described herein.

According to another aspect of the present invention, there is provided a seed, fruit or a leaf obtained from a plant as described herein.

According to another aspect of the present invention, there is provided triacylglycerol produced from a non-human organism or a cell thereof as described herein. Preferably, there is provided triacylglycerol produced from a plant, preferably a seed, fruit or a leaf of a plant as described herein.

Preferably, the triacylglycerol comprises more than about 30% of the C16:0 at the sn-2 position, preferably more than about 35%, preferably more than about 40%, preferably more than about 45%, preferably more than about 50%, preferably more than about 55%, preferably more than about 60%, preferably more than about 65%, most preferably more than about 70% of the C16:0 at the sn-2 position.

Preferably, the triacylglycerol comprises between about 30% and about 100% of the C16:0 at the sn-2 position, preferably between about 35% and about 100%, preferably between about 40% and about 100%, preferably between about 45% and about 100%, preferably between about 50% and about 100%, preferably between about 55% and about 100%, preferably between about 60% and about 100%, preferably between about 65% and about 100%, most preferably between about 70% and about 100% of the C16:0 at the sn-2 position.

Preferably the organism is a plant and the triacylglycerol is obtained from a seed, fruit and/or a leaf of the plant.

According to another aspect of the invention, there is provided a method for extracting triacylglycerol from an organism as described herein, preferably a plant as described herein, preferably wherein the method comprises mechanical extraction and/or solvent extraction.

According to another aspect of the invention, there is provided a method for making triacylglycerol from a yeast as described herein, the method comprising culturing said yeast and extracting triacylglycerol therefrom.

In another aspect of the present invention, there is provided a method for producing an infant formula, comprising obtaining triacylglycerol from a non-human organism or a cell thereof as described herein and using said triacylglycerol to produce an infant formula.

Preferably, the organism is a plant and the triacylglycerol is extracted from the plant.

Preferably, the triacylglycerol is extracted from a seed, fruit and/or a leaf of the plant.

According to another aspect of the present invention, there is provided infant formula comprising triacylglycerol as described herein and/or produced from a method as described herein.

According to another aspect of the present invention, there is provided a non-human organism comprising a C16:0 LPAT, wherein the organism is cultured in a media comprising a carbon source, wherein the carbon source comprises a mixture of (i) one or more sugars and (ii) one or more fatty acids and/or fatty acid esters.

Preferably, the carbon source comprises a mixture of (i) one or more sugars and (ii) one or more oils, preferably vegetable oils.

Preferably, the one or more sugars comprise one or more fermentable sugars.

Preferably, the one or more sugars is selected from one or more of xylose, lactose, cellulose, glucose, fructose, sucrose, or hydrolysed lignocellulosic materials.

Preferably, the one or more fatty acids and/or fatty acid esters comprises C16:0.

Preferably, the one or more fatty acids and/or fatty acid esters comprises a mixture of fatty acids and/or fatty acid esters wherein at least about 30% w/w of the fatty acids and/or fatty acid esters comprises C16:0 and/or at least about 30% w/w of the fatty acids and/or fatty acid esters comprises C18:1.

Preferably, the carbon source comprises palm oil in combination with glucose and/or glycerol.

Preferably, the organism is treated or genetically modified to express a C16:0 LPAT.

Preferably, the C16:0 LPAT is a heterologous C16:0 LPAT.

As will be appreciated, the triacylglycerol forms part of the infant formula as an ingredient therein.

Preferably, the infant formula comprises one or more additional ingredients. Preferably, the one or more additional ingredients include one or more of water, lactose, emulsifiers, pre-biotics, pro-biotics, vitamins and/or minerals.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

### DETAILED DESCRIPTION

Example embodiments of the present invention will now be described with reference to the accompanying Figures, in which

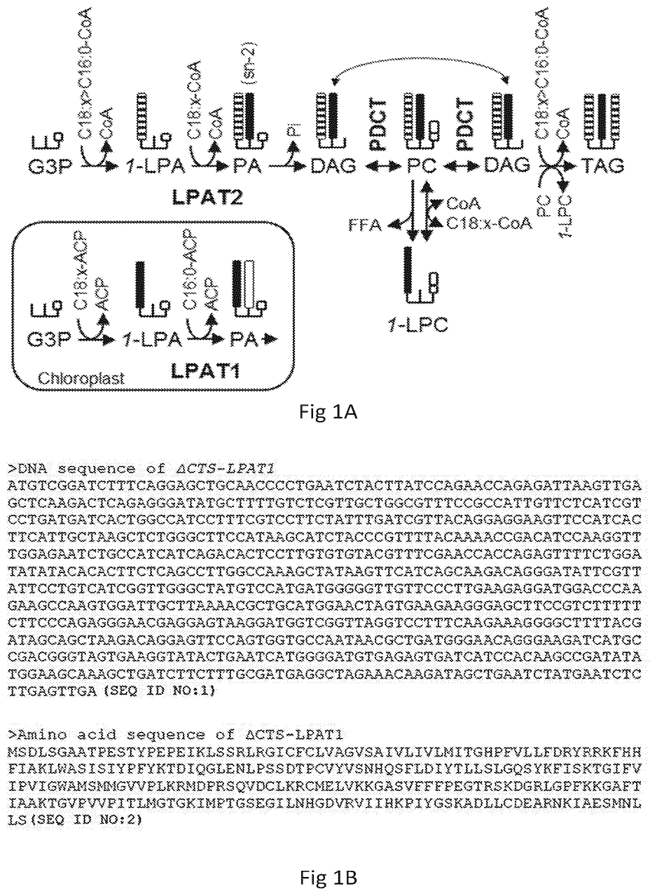

FIG. 1A shows a diagram illustrating the cytosolic and chloroplastic pathways for de novo glycerolipid biosynthesis in Arabidopsis. Three modifications enabled palmitoyl (C16:0) groups (white bars) to be incorporated into the sn-2 (or β) position of TAG in developing seeds. (1) Retargeting of LPAT1 to the ER, (2) knock down of LPAT2 and (3) knock out of PDCT. C18:x, long-chain mono- or polyunsaturated fatty acyl groups (black bars); CoA, Coenzyme A; ACP, acyl carrier protein; G3P, glycerol-3-phosphate; 1-LPA, sn-1 lysophosphatidic acid; PA, phosphatidic acid, DAG, diacylglycerol, TAG, triacylglycerol; PC, phosphatidylcholine; 1-LPC, sn-1 lysophosphatidylcholine; FFA, free fatty acid; LPAT, 1-LPA acyltransferase; PDCT, PC:DAG cholinephosphotransferase;

FIG. 1B shows the DNA sequence (SEQ ID NO:1) and the amino acid sequence (SEQ ID NO:2) of ΔCTS-LPAT1;

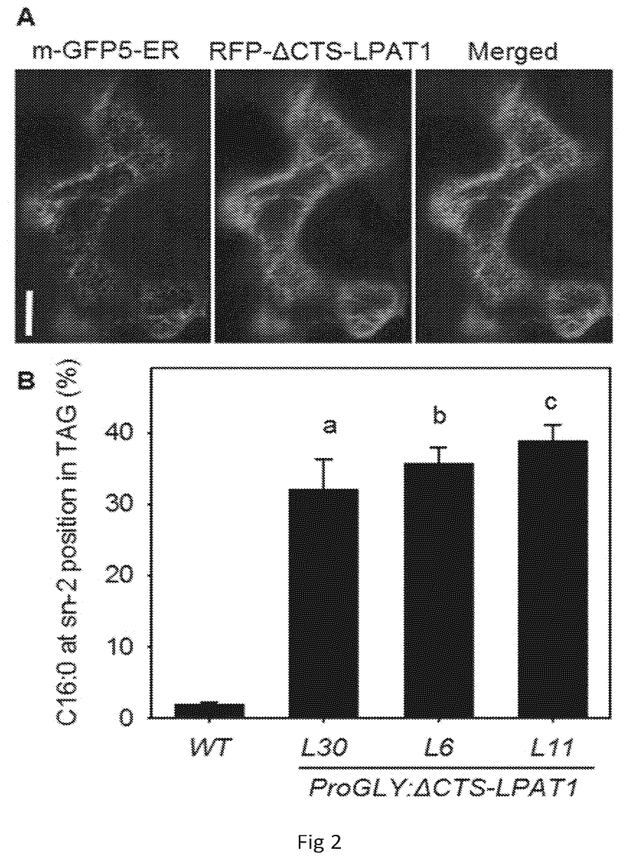

FIG. 2 shows that chloroplast LPAT1 can be retargeted to the cytosolic glycerolipid biosynthetic pathway to incorporate C16:0 into the sn-2 position of TAG. (A) Laser scanning confocal microscopy image of a N. benthamiana epidermal cell transiently expressing RFP-ΔCTS-LPAT1 and m-GFP5-ER marker. Scale bar=20 μm. (B) Effect of seed-specific ΔCTS-LPAT1 expression in Arabidopsis on the percentage of C16:0 esterified to the sn-2 position of TAG, verses sn-1+3. WT=wild type; L30, L6 and L11=three independent homozygous ProGLY:ΔCTS-LPAT1 lines. Values are the mean±SE of measurements made on separate seed batches from three plants of each genotype (n=3). a, b and c denote values significantly (P<0.05) different from WT (ANOVA+Tukey HSD test);

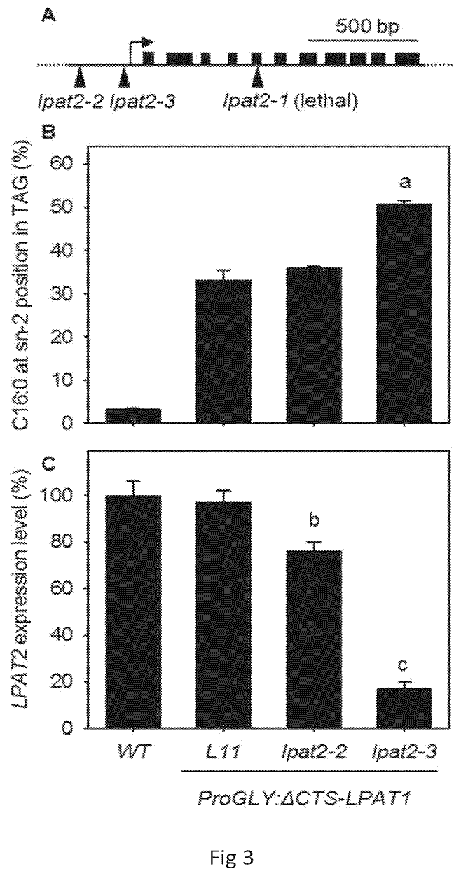

FIG. 3 shows disruption of ER-resident LPAT2 increases C16:0 incorporation into the sn-2 position of TAG. (A) Diagram of LPAT2 locus showing positions of T-DNA insertions in mutant alleles. Effect of lpat2 mutant backgrounds on (B) the percentage of C16:0 esterified to the sn-2 position of TAG, verses sn-1+3, and (C) LPAT2 transcript abundance in seeds expressing ΔCTS-LPAT1. WT=wild type; L11=homozygous ProGLY:ΔCTS-LPAT1 line. Values are the mean±SE of measurements made on separate batches of dry seeds in B and developing siliques in C from three plants of each genotype (n=3). LPAT2 expression was normalised to the geometric mean of three reference genes and expressed relative to WT. a, b & c denote values significantly (P<0.05) different from L11 (ANOVA+Tukey HSD test);

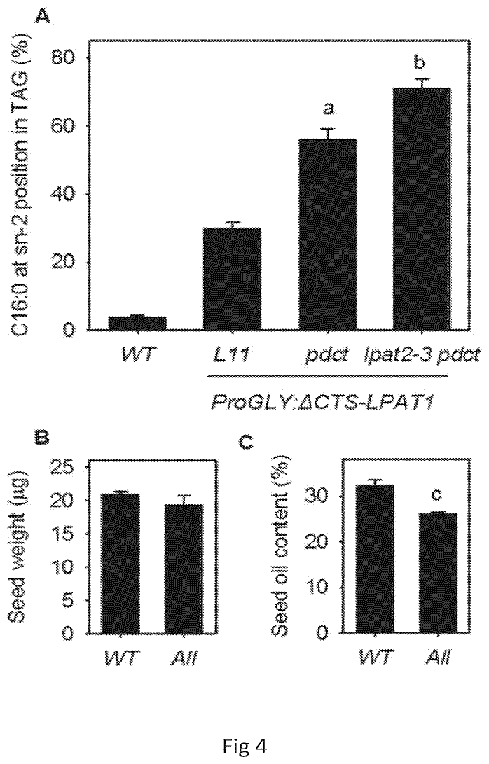

FIG. 4 shows that bypassing flux through PC increases C16:0 incorporation into the sn-2 position of TAG. (A) Effect of pdct mutant background on percentage of C16:0 esterified to the sn-2 position of TAG in ProGLY:ΔCTS-LPAT1 and ProGLY:ΔCTS-LPAT1 lpat2-3 seeds. WT=wild type; L11=homozygous ProGLY:ΔCTS-LPAT1 line. (B) Seed weight and (C) percentage oil content of WT and ProGLY:ΔCTS-LPAT1 lpat2-3 pdct (All). Values are the mean±SE of measurements on separate seed batches from between three and six plants in A and five plants in B and C of each genotype (n=3 to 6). a and b denote values significantly (P<0.05) different from L11 and pdct, respectively (ANOVA+Tukey HSD test) and c from WT (two-tailed Student's t test);

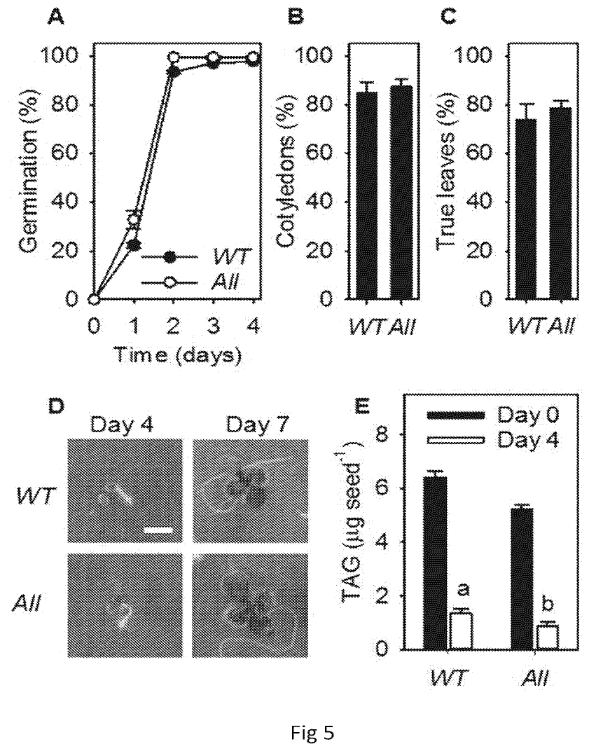

FIG. 5 shows the effect of genetic modifications on seed vigour. Percentage (A) seed germination, (B) cotyledons expanded by day 4 and (C) true leaves developing by day 7. (D) Representative images of seedlings with expanded cotyledons and developing true leaves. (E) Seed/seedling TAG content at day 0 and 4. WT=wild type; All=ProGLY:ΔCTS-LPAT1 lpat2-3 pdct. Values are the mean±SE of measurements made on separate seed batches from three plants of each genotype (n=3). In D, scale bar=2 mm. a and b denote values significantly (P<0.05) different from WT (two-tailed Student's t tests);

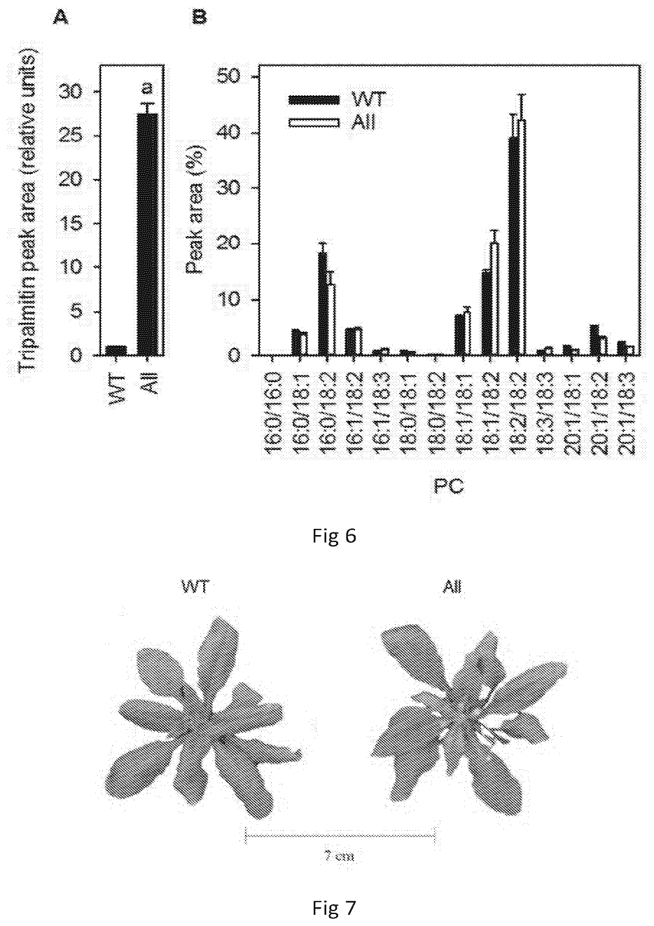

FIG. 6 shows the effect of genetic modifications on seed lipid composition. (A) Tripalmitin content and (B) PC composition. WT=wild type; All=ProGLY:ΔCTS-LPAT1 lpat2-3 pdct. Values are the mean±SE of measurements made on separate seed batches from five plants of each genotype (n=5). a denotes a value significantly (P<0.05) different from WT (two-tailed Student's t tests);

FIG. 7 shows images of wild type (WT) and ProGLY:ΔCTS-LPAT1 lpat2-3 pdct (All) plants at onset of flowering;

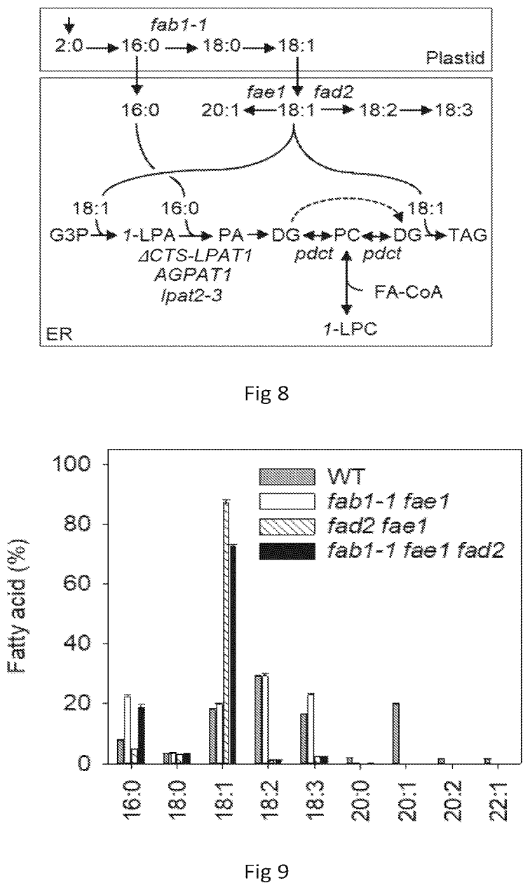

FIG. 8 shows a simplified diagram illustrating the strategy used to produce OPO in Arabidopsis seeds. A combination of the hypomorphic fab1-1 and null fae1 and fad2 mutant alleles was used to produce high levels of 16:0 and 18:1 in seeds. Expression of an ER-localised LPAT with 16:0 -CoA preference combined with a hypomorphic lpat2-3 and null pdct mutant allele was then used to enable 16:0 to be preferentially esterified to the sn-2 position of 1-LPA and the products channelled into TG. 16:0, palmitic acid; 18:1, oleic acid; CoA, Coenzyme A; G3P, glycerol-3-phosphate; 1-LPA, sn-1 lysophosphatidic acid; PA, phosphatidic acid, DG, diacylglycerol, TAG, triacylglycerol; PC, phosphatidylcholine; 1-LPC, sn-1 lysophosphatidylcholine; FA, fatty acid;

FIG. 9 shows fatty acid composition of TAG from HPHO seeds. Total fatty acid composition of TAG isolated from WT, fab1-1 fae1, fad2 fae1 and fab1-1 fae1 fad2 seeds. Values are the mean±SE of measurements on seeds from three plants of each genotype;

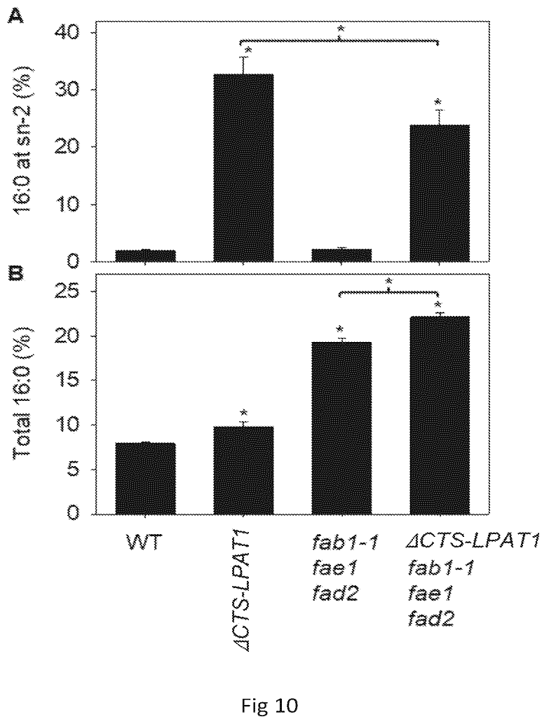

FIG. 10 shows 16:0 in TAG from WT and HPHO seeds expressing ΔCTS-LPAT1. Percentage of 16:0 esterified to the sn-2 position (A) and 16:0 as a percentage of total fatty acid content (B) measured in TAG isolated from WT, ProGLY:ΔCTS-LPAT1, fab1-1 fad2 fae1 and ProGLY:ΔCTS-LPAT1 fab1-1 fad2 fae1 seeds. Values are the mean±SE of measurements on seeds from three plants of each genotype. * denote values significantly (P<0.05) different either from WT or, where marked in parenthesis, from one another (ANOVA+Tukey HSD test);

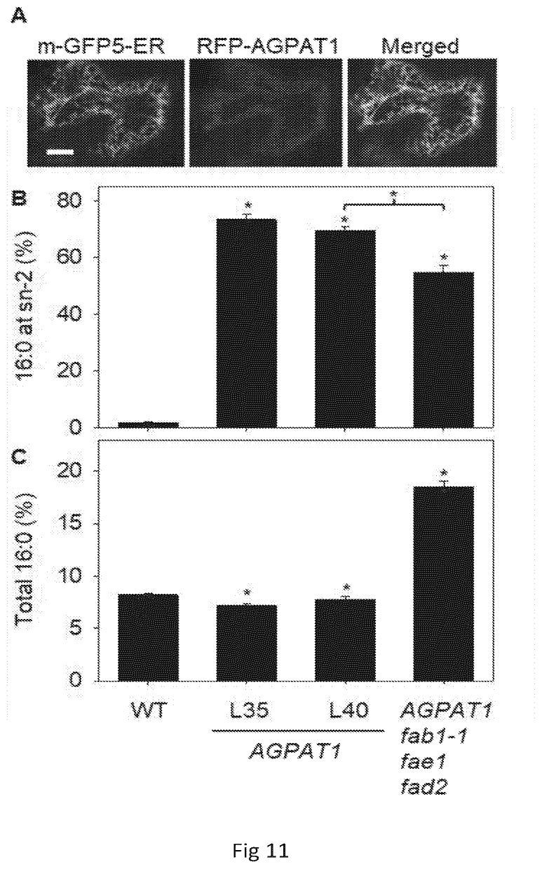

FIG. 11 shows 16:0 in TAG from WT and HPHO seeds expressing AGPAT1. Laser scanning confocal microscopy image of a N. benthamiana epidermal cell transiently expressing RFP-AGPAT1and m-GFP5-ER marker (A). Scale bar=20 μm. Percentage of 16:0 esterified to the sn-2 position (B) and 16:0 as a percentage of total fatty acid content (C) measured in TAG isolated from WT, ProGLY:AGPAT1, fab1-1 fad2 fae1 and ProGLY:AGPAT1 fab1-1 fad2 fae1 seeds. Values are the mean±SE of measurements on seeds from three plants of each genotype. * denote values significantly (P<0.05) different either from WT or, where marked in parenthesis, from one another (ANOVA+Tukey HSD test);

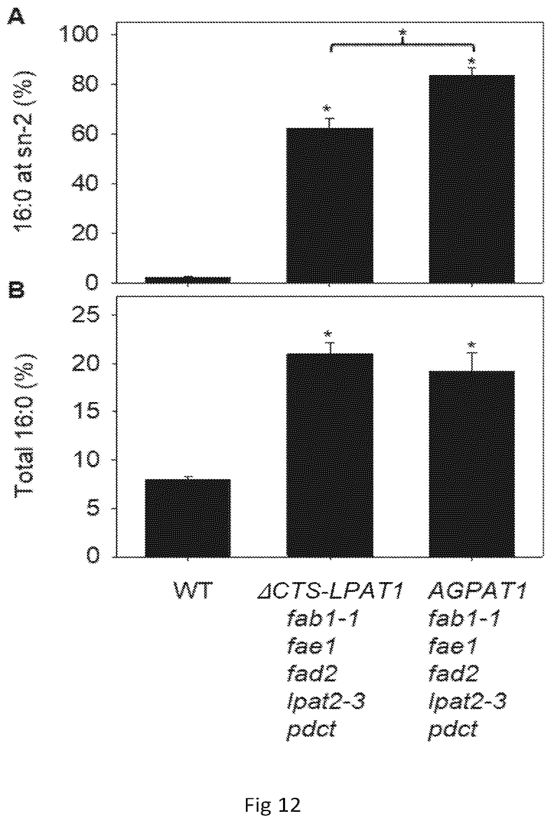

FIG. 12 shows 16:0 in TAG from HPHO lpat2-3 pdct seeds expressing ΔCTS-LPAT1 or AGPAT1. Percentage of 16:0 esterified to the sn-2 position (A) and 16:0 as a percentage of total fatty acid content (B) measured in TAG isolated from WT, ProGLY:ΔCTS-LPAT1 fab1-1 fae1 fad2, lpat2-3 pdct and ProGLY:AGPAT1 fab1-1 fae1 fad2 lpat2-3 pdct seeds. Values are the mean±SE of measurements on seeds from three plants of each genotype. * denote values significantly (P<0.05) different either from WT or, where marked in parenthesis, from one another (ANOVA+Tukey HSD test);

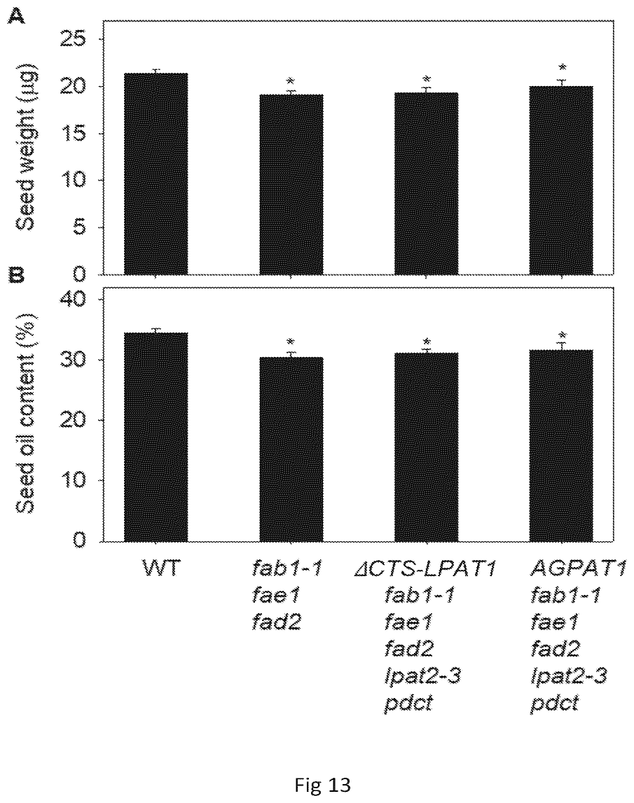

FIG. 13 shows oil content of HPHO lpat2-3 pdct seeds expressing ΔCTS-LPAT1 or AGPAT1. Seed weight (A) and percentage oil content (B) of WT, fab1-1 fae1 fad2, ProGLY:ΔCTS-LPAT1 fab1-1 fae1 fad2, lpat2-3 pdct and ProGLY:AGPAT1 fab1-1 fae1 fad2 lpat2-3 pdct seeds. Values are the mean±SE of measurements on seeds from three plants of each genotype. * denote values significantly (P<0.05) different either from WT (ANOVA+Tukey HSD test); and

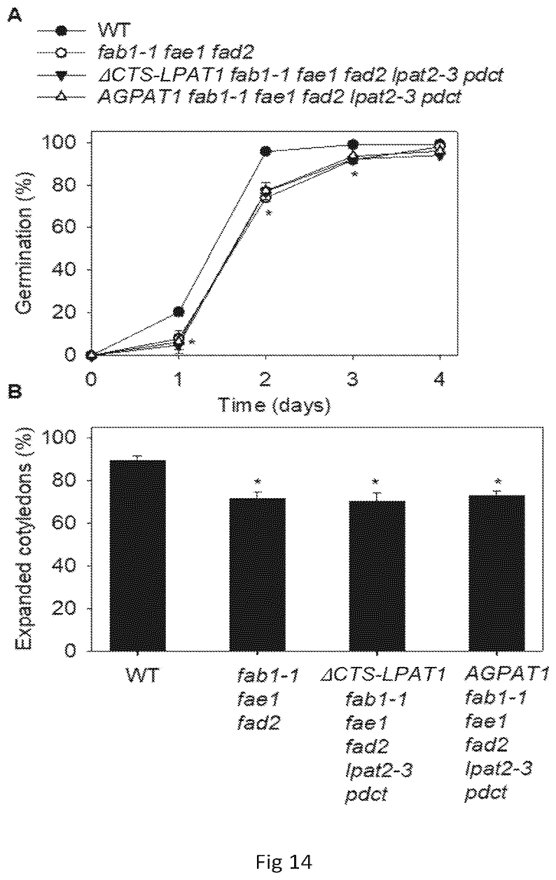

FIG. 14 shows vigour of HPHO lpat2-3 pdct seeds expressing ΔCTS-LPAT1 or AGPAT1. Percentage seed germination (A) and cotyledons fully expanded by day 4 (B) of WT, fab1-1 fae1 fad2, ProGLY ΔCTS-LPAT1 fab1-1 fae1 fad2, lpat2-3 pct and ProGLY:AGPAT1 fab1-1 fae1 fad2 lpat2-3 pdct seeds. Values are the mean±SE of measurements on seeds from three plants of each genotype. * denote values significantly (P<0.05) different either from WT (ANOVA+Tukey HSD test).

The present invention relates to a non-human organism for producing triacylglycerol in which the fatty acid stereoisomeric structure of human triacylglycerol is mimicked. In particular, the invention provides non-human organisms which produce triacylglycerol with a similar percentage of C16:0 at the sn-2 position of the glycerol backbone to that of human triacylglycerol.

The aim of the invention described herein was to explore whether the stereoisomeric structure of vegetable oil can be altered by iterative metabolic engineering, so that it mimics HMF. To our knowledge, no land plant (Embryophyta) produces TAG enriched in C16:0 at the sn-2 (verses sn-1/3 positions) and C16:0 is largely excluded from this position in virtually all cases (4,5,8). Even in palm oil that contains ˜48% C16:0 in total, only 9% of this occupies the sn-2 position. Described herein is a method for modifying TAG biosynthesis, in the model oilseed Arabidopsis thaliana, that results in a stereoisomeric redistribution of acyl groups such that the amount of C16:0 at the sn-2 position increases more than 20-fold to over 70% of the total; a level of enrichment that is comparable to HMF. It is envisaged that applying this technology to oilseed crops will provide a cheaper and therefore more widely accessible source of HMFS for infant formula, given that it could abrogate the need for enzyme-based catalysis.

Within this specification, the term “about” means plus or minus 20%, more preferably plus or minus 10%, even more preferably plus or minus 5%, most preferably plus or minus 2%.

Within this specification, the term “a lysophosphatidic acid acyltransferase specific for C16:0-Coenzyme A” means a lysophosphatidic acid acyltransferase which esterifies C16:0 to the sn-2 position on the glycerol backbone in preference to other fatty acyl-CoA substrates, preferably in preference to longer chain unsaturated fatty acyl-CoAs. Put another way, “a lysophosphatidic acid acyltransferase specific for C16:0-Coenzyme A” has greater activity using C16:0-Coenzyme A than other fatty acyl-CoAs, preferably than longer chain unsaturated fatty acyl-CoAs.

Preferably, the term “a lysophosphatidic acid acyltransferase specific for C16:0-Coenzyme A” means a lysophosphatidic acid acyltransferase which esterifies C16:0 to the sn-2 position on the glycerol backbone of sn-1 lysophosphatidic acid in preference to using fatty acyl-CoA substrates with acyl groups of more than 16 carbon atoms chain length and containing one or more double bonds.

Within this specification, the term “a lysophosphatidic acid acyltransferase which is not specific for C16:0-Coenzyme A” means a lysophosphatidic acid acyltransferase which does not esterify C16:0 to the sn-2 position on the glycerol backbone in preference to other fatty acyl-CoA substrates, preferably in preference to longer chain unsaturated fatty acyl-CoAs. Put another way, “a lysophosphatidic acid acyltransferase which is not specific for C16:0-Coenzyme A” has lower activity using C16:0-Coenzyme A than other fatty acyl-CoAs, preferably than longer chain unsaturated fatty acyl-CoAs.

Preferably, the term “a lysophosphatidic acid acyltransferase which is not specific for C16:0-Coenzyme A” means a lysophosphatidic acid acyltransferase which does not esterify C16:0 to the sn-2 position on the glycerol backbone of sn-1 lysophosphatidic acid in preference to using fatty acyl-CoA substrates with acyl groups of more than 16 carbon atoms and containing one or more double bonds.

Within this specification, “identity,” as it is known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. Percentage identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SLAM J. Applied Math., 48:1073 (1988), all of which are incorporated herein by reference in their entirety. Preferred methods to determine identity are designed to give the largest match between the sequences tested. Methods to determine identity are codified in publicly available computer programs. Preferred computer program methods to determine percentage identity between two sequences include, but are not limited to, the GCG program package (Devereux, J., et al., Nucleic Acids Research 12(1):387 (1984), which is incorporated herein by reference in its entirety), BLASTP, BLASTN, and FASTA (Atschul, S. F. et al., J. Molec. Biol. 215:403-410 (1990), which is incorporated herein by reference in its entirety). The BLAST X program is publicly available from NCBI and other sources (BLAST Manual, Altschul, S., et al, NCBI NLM NIH Bethesda, Md. 20894; Altschul, S., et al., J. Mol. Biol. 215:403-410 (1990), which is incorporated herein by reference in its entirety). As an illustration, by a polynucleotide having a nucleotide sequence having at least, for example, 95% “identity” to a reference nucleotide sequence of “SEQ ID NO: A” it is intended that the nucleotide sequence of the polynucleotide is identical to the reference sequence except that the polynucleotide sequence may include up to five point mutations per each 100 nucleotides of the reference nucleotide sequence of “SEQ ID NO: A.” In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid sequence of “SEQ ID NO: B” is intended that the amino acid sequence of the polypeptide is identical to the reference sequence except that the polypeptide sequence may include up to five amino acid alterations per each 100 amino acids of the reference amino acid of “SEQ ID NO: B.” In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

With reference to the sequence identity described herein, it will be appreciated that many of the non-human organisms to which the present invention relates are polyploid. This means that they can have multiple copies of each gene with small variations in sequence identity. It will, therefore, be appreciated that reference to a particular gene or protein described herein also includes reference to one or more additional copies of that gene and associated encoded protein from the same organism.

Preferably, the non-human organism is polyploid and reference to a particular sequence referred to herein includes reference to a sequence having at least about 80% sequence identity to said sequence, preferably at least about 85% sequence identity, preferably at least about 90% sequence identity, preferably at least about 95% sequence identity, preferably at least about 98% sequence identity, preferably at least about 99% sequence identity to said sequence.

It will be appreciated that reference to “one or more” includes reference to “a plurality”.

Within this specification, “genetically modified” means an organism in which the DNA of the organism has been modified. This could, for example, be by introducing DNA from another organism or by modifying the existing DNA.

### Example 1

### Results and Discussion

### LPAT1 Can Be Redirected to the ER by Removing its Chloroplast Targeting Signal

In plant cells, triacylglycerol (TAG) is formed by a cytosolic glycerolipid biosynthetic pathway situated on the endoplasmic reticulum (ER) and the enzyme responsible for acylation of the sn-2 position is lysophosphatidic acid acyltransferase (LPAT) (FIG. 1A). ER-resident isoforms of LPAT can discriminate against C16:0-Coenzyme A (CoA) as a substrate and this might be why C16:0 is excluded from the sn-2 position. To overcome this limitation, we decided to express an LPAT with specificity for C16:0-CoA (FIG. 1A). Several candidate transgenes have been described from cyanobacteria, mammals and algae. However, plants already possess an LPAT with the appropriate selectivity, that resides in the chloroplast (FIG. 1A). This LPAT uses a C16:0-acyl carrier protein (ACP) substrate but will also accept C16:0-CoA in vitro. We therefore decided to test whether chloroplast LPAT could be relocated to the ER (FIG. 1A). Chloroplast LPAT is an integral membrane protein that is nuclear encoded and contains an N-terminal chloroplast targeting signal (CTS). Using transient expression in Nicotiana benthamiana leaves, we found that when 101 amino acid residues containing the CTS are deleted from Brassica napus LPAT1 (FIG. 1B) and replaced with a red fluorescent protein (RFP) marker the RFP-ΔCTS-LPAT1 fusion protein localises to the ER (FIG. 2A).

ΔCTS-LPAT1 Expression Drives C16:0 Incorporation Into the sn-2 Position of TAG

Truncated versions of LPAT1 that lack the CTS are known to be active when expressed in Escherichia coli. To determine whether ΔCTS-LPAT1 functions in plants and can enable C16:0 to be incorporated into the sn-2 position of TAG, we expressed this truncated protein under the control of the seed-specific soybean glycinin-1 promoter (ProGLY) in the model oilseed Arabidopsis thaliana. We selected more than forty primary transformants (T1) using a DsRed fluorescent marker system and analysed the total fatty acyl composition of T2 seed batches. We found that several lines exhibited an increase in total C16:0 content, which suggested that the transgene was promoting C16:0 incorporation into TAG (Table 1).

TABLE 1
Total fatty acid composition of seed batches from individual
wild type (WT) and segregating T2 ProGLY:ΔCTS-LPAT1
lines (L1-42). Asterisk denotes single copy lines taken
forward for further analysis.

| | Fatty acid content (%) | | | | | |
|---|---|---|---|---|---|---|
| Line | 16:0 | 18:0 | 18:1 | 18:2 | 18:3 | 20:1 |
| WT 1 | 8.00 | 2.78 | 13.90 | 30.90 | 22.67 | 22.01 |
| WT 2 | 8.22 | 2.89 | 13.98 | 30.61 | 22.82 | 21.74 |
| WT 3 | 8.25 | 2.89 | 13.55 | 30.63 | 23.06 | 21.88 |
| WT 4 | 8.01 | 2.92 | 14.42 | 30.68 | 22.47 | 21.75 |
| WT 5 | 8.16 | 2.60 | 13.91 | 31.08 | 22.23 | 22.27 |
| L1 | 7.83 | 3.67 | 18.16 | 31.16 | 18.99 | 20.19 |
| L2 | 9.27 | 3.42 | 17.90 | 31.03 | 19.03 | 19.34 |
| L3 | 8.28 | 3.20 | 16.88 | 31.52 | 19.19 | 20.93 |
| L4 | 8.92 | 3.99 | 17.69 | 30.77 | 19.33 | 19.31 |
| L5 | 8.15 | 4.06 | 18.24 | 32.40 | 18.13 | 19.02 |
| L6* | 9.97 | 3.55 | 17.16 | 30.69 | 20.03 | 18.60 |
| L7 | 7.84 | 3.16 | 17.09 | 31.15 | 20.44 | 20.33 |
| L8 | 7.72 | 3.21 | 17.71 | 31.37 | 20.14 | 19.86 |
| L9 | 8.47 | 3.43 | 17.85 | 30.87 | 19.51 | 19.88 |
| L10 | 9.62 | 3.50 | 18.78 | 30.25 | 19.99 | 17.86 |
| L11* | 10.67 | 3.44 | 12.91 | 33.47 | 21.74 | 17.77 |
| L13 | 8.63 | 2.50 | 15.27 | 31.12 | 21.88 | 20.61 |
| L14 | 7.65 | 2.97 | 17.26 | 30.70 | 21.25 | 20.18 |
| L15 | 8.43 | 3.42 | 17.09 | 30.41 | 20.35 | 20.30 |
| L16 | 7.92 | 3.34 | 18.25 | 31.03 | 19.90 | 19.55 |
| L17 | 9.73 | 3.05 | 15.59 | 31.22 | 21.84 | 18.58 |
| L18 | 7.95 | 3.44 | 18.56 | 30.79 | 19.82 | 19.44 |
| L19 | 8.22 | 3.38 | 17.62 | 30.97 | 20.17 | 19.65 |
| L20 | 7.62 | 3.61 | 18.74 | 31.27 | 20.16 | 18.60 |
| L21 | 7.77 | 3.47 | 17.55 | 30.67 | 20.70 | 19.84 |
| L22 | 8.20 | 3.37 | 19.47 | 31.89 | 17.86 | 19.21 |
| L23 | 7.81 | 3.12 | 18.98 | 30.55 | 19.32 | 20.22 |
| L24 | 7.64 | 3.67 | 18.94 | 30.96 | 19.11 | 19.69 |
| L25 | 9.06 | 2.64 | 12.92 | 33.09 | 21.56 | 20.74 |
| L26 | 9.64 | 2.79 | 20.64 | 28.78 | 19.87 | 18.29 |
| L27 | 7.83 | 3.50 | 18.73 | 31.22 | 19.15 | 19.56 |
| L28 | 7.62 | 3.59 | 19.73 | 31.55 | 18.56 | 18.96 |
| L29 | 7.45 | 3.72 | 19.45 | 31.55 | 18.94 | 18.89 |
| L30* | 9.88 | 3.57 | 19.36 | 30.37 | 19.14 | 17.68 |
| L31 | 7.60 | 3.53 | 18.40 | 31.11 | 19.59 | 19.77 |
| L32 | 9.19 | 3.64 | 18.45 | 31.83 | 18.60 | 18.30 |
| L33 | 7.71 | 3.76 | 18.37 | 31.26 | 19.65 | 19.25 |
| L34 | 7.56 | 3.62 | 19.03 | 30.69 | 19.46 | 19.64 |
| L35 | 7.36 | 3.61 | 20.01 | 31.15 | 18.32 | 19.55 |
| L36 | 8.81 | 3.64 | 18.75 | 31.23 | 18.99 | 18.58 |
| L37 | 7.55 | 3.78 | 19.42 | 31.55 | 18.46 | 19.24 |
| L38 | 7.34 | 3.99 | 23.01 | 30.95 | 15.89 | 19.82 |
| L39 | 8.07 | 3.70 | 19.15 | 31.74 | 18.50 | 18.84 |
| L41 | 9.59 | 3.84 | 18.05 | 31.58 | 19.13 | 17.82 |
| L42 | 7.80 | 3.46 | 18.69 | 30.51 | 21.11 | 19.43 |

We selected three independent single copy T2 lines (L30, L6 and L11) with high C16:0 content and obtained homozygous T3 seed. When we purified TAG from these homozygous seed batches and determined its stereochemistry using lipase digestion, we found that the percentage of C16:0 at the sn-2 position (versus sn-1+3), had increased more than 16-fold, from only ˜2% in wild type to values ranging between ˜32 and ˜39% in the three independent ProGLY:ΔCTS-LPAT1 lines (FIG. 2B; Table 2).

TABLE 2
Total and sn-2 fatty acyl composition of TAG from wild type
(WT) and homozygous ProGLY:ΔCTS-LPAT1 seeds.

| | Fatty acid content (%) | | | | | | | | |
|---|---|---|---|---|---|---|---|---|---|
| Line | 16:0 | 18:0 | 18:1 | 18:2 | 18:3 | 20:0 | 20:1 | 20:2 | 22:1 |
| Total | | | | | | | | | |
| WT 1 | 8.23 | 4.17 | 17.56 | 28.10 | 15.87 | 1.97 | 21.02 | 1.63 | 1.36 |
| WT 2 | 8.14 | 4.05 | 18.19 | 28.06 | 16.31 | 2.01 | 20.21 | 1.62 | 1.42 |
| WT 3 | 8.31 | 3.99 | 16.46 | 28.77 | 16.48 | 2.10 | 20.53 | 1.83 | 1.53 |
| L30 1 | 9.39 | 3.08 | 17.75 | 29.03 | 16.15 | 1.90 | 19.49 | 1.56 | 1.64 |
| L30 2 | 10.26 | 3.27 | 19.08 | 28.37 | 15.43 | 1.95 | 18.89 | 1.37 | 1.38 |
| L30 3 | 8.53 | 3.24 | 18.90 | 28.24 | 15.23 | 1.94 | 20.80 | 1.48 | 1.64 |
| L6 1 | 8.55 | 3.08 | 17.70 | 28.49 | 16.28 | 1.85 | 20.74 | 1.64 | 1.67 |
| L6 2 | 9.58 | 3.26 | 17.94 | 28.43 | 16.32 | 1.95 | 19.54 | 1.53 | 1.53 |
| L6 3 | 8.04 | 2.77 | 17.50 | 29.24 | 16.26 | 1.70 | 20.94 | 1.74 | 1.74 |
| L11 1 | 7.15 | 3.15 | 16.97 | 27.94 | 18.17 | 2.43 | 20.75 | 1.93 | 1.50 |
| L11 2 | 7.68 | 3.29 | 16.20 | 26.98 | 15.14 | 2.14 | 24.81 | 2.19 | 1.30 |
| L11 3 | 7.57 | 3.27 | 15.79 | 26.20 | 18.70 | 2.13 | 21.95 | 2.46 | 1.93 |
| sn-2 | | | | | | | | | |
| WT 1 | 0.53 | 0.25 | 18.07 | 53.05 | 28.10 | 0.00 | 0.00 | 0.00 | 0.00 |
| WT 2 | 0.33 | 0.24 | 18.49 | 52.52 | 28.43 | 0.00 | 0.00 | 0.00 | 0.00 |
| WT 3 | 0.60 | 0.53 | 16.70 | 54.30 | 27.87 | 0.00 | 0.00 | 0.00 | 0.00 |
| L30 1 | 9.22 | 0.30 | 16.65 | 49.81 | 24.03 | 0.00 | 0.00 | 0.00 | 0.00 |
| L30 2 | 7.50 | 0.64 | 19.15 | 49.70 | 23.01 | 0.00 | 0.00 | 0.00 | 0.00 |
| L30 3 | 10.01 | 0.64 | 17.79 | 48.73 | 22.83 | 0.00 | 0.00 | 0.00 | 0.00 |
| L6 1 | 10.17 | 0.68 | 15.88 | 49.23 | 24.04 | 0.00 | 0.00 | 0.00 | 0.00 |
| L6 2 | 9.21 | 0.26 | 17.24 | 48.90 | 24.40 | 0.00 | 0.00 | 0.00 | 0.00 |
| L6 3 | 8.59 | 0.75 | 15.53 | 50.71 | 24.42 | 0.00 | 0.00 | 0.00 | 0.00 |
| L11 1 | 8.49 | 0.37 | 14.99 | 48.02 | 28.13 | 0.00 | 0.00 | 0.00 | 0.00 |
| L11 2 | 9.77 | n.d. | 14.34 | 48.84 | 27.06 | 0.00 | 0.00 | 0.00 | 0.00 |
| L11 3 | 7.88 | 1.17 | 14.76 | 47.08 | 29.12 | 0.00 | 0.00 | 0.00 | 0.00 |

ΔCTS-LPAT1 expression was therefore sufficient to allow incorporation of C16:0 into the sn-2 position of TAG, but not to achieve positive enrichment at this position verses the sn-1/3 positions, which can already incorporate a low proportion of C16:0 (FIG. 1A).

Disruption of LPAT2 Enhances C16:0 Incorporation Into the sn-2 Position of TAG

Competition between heterologous and native acyltransferases might be a factor that can limit the incorporation of specific fatty acyl groups into TAG. We therefore investigated whether ΔCTS-LPAT1-dependent incorporation of C16:0 into the sn-2 position of TAG could be enhanced by disrupting the function of the native ER-resident LPAT; believed to be LPAT2 in Arabidopsis (FIG. 1A). The lpat2-1 null mutant is embryo lethal. However, T-DNA insertions in non-coding regions of essential genes can be used to produce viable hypomorphic alleles. We therefore isolated two T-DNA mutants (lpat2-2 and lpat2-3) with insertions 302 and 139 bp 5′ of the LPAT2 translational start site (FIG. 3A). We then crossed ProGLY:ΔCTS-LPAT1 L11 into each of the new lpat2 alleles and recovered homozygous seed batches. When we purified TAG from these seed batches and performed positional analysis, we found that the percentage of C16:0 at the sn-2 position had increased from ˜33% in the parental ProGLY:ΔCTS-LPAT1 line to ˜51% in the lpat2-3 background, whereas the effect in the lpat2-2 background was not significant (P>0.05) (FIG. 3B; Table 3).

TABLE 3
Total and sn-2 fatty acyl composition of TAG from wild type (WT) and
homozygous ProGLY:ΔCTS-LPAT1 seeds containing lpat2 mutant alleles.

| | Fatty acid content (%) | | | | | | | | |
|---|---|---|---|---|---|---|---|---|---|
| Line | 16:0 | 18:0 | 18:1 | 18:2 | 18:3 | 20:0 | 20:1 | 20:2 | 22:1 |
| Total | | | | | | | | | |
| WT 1 | 7.39 | 3.51 | 18.41 | 29.26 | 16.08 | 2.00 | 20.41 | 1.70 | 1.25 |
| WT 2 | 7.53 | 3.44 | 17.51 | 29.13 | 16.69 | 2.02 | 20.32 | 1.80 | 1.56 |
| WT 3 | 7.96 | 3.41 | 17.68 | 29.31 | 16.91 | 1.92 | 19.64 | 1.72 | 1.46 |
| L11 1 | 8.58 | 3.45 | 13.18 | 26.68 | 19.35 | 2.73 | 22.10 | 2.11 | 1.82 |
| L11 2 | 9.03 | 2.79 | 15.48 | 27.50 | 20.67 | 1.90 | 19.16 | 1.84 | 1.63 |
| L11 3 | 8.49 | 2.87 | 15.33 | 27.07 | 20.89 | 1.96 | 20.00 | 1.89 | 1.50 |
| L11 | 8.69 | 3.29 | 15.66 | 26.67 | 19.76 | 2.10 | 20.58 | 1.82 | 1.44 |
| lpat2-2 1 | | | | | | | | | |
| L11 | 9.28 | 3.31 | 15.51 | 27.53 | 19.74 | 1.94 | 19.49 | 1.80 | 1.40 |
| lpat2-2 2 | | | | | | | | | |
| L11 | 8.58 | 2.77 | 13.18 | 29.05 | 20.04 | 2.30 | 20.56 | 2.02 | 1.50 |
| lpat2-2 3 | | | | | | | | | |
| L11 | 9.04 | 3.17 | 18.75 | 27.28 | 15.90 | 1.64 | 21.58 | 1.28 | 1.36 |
| lpat2-3 1 | | | | | | | | | |
| L11 | 9.08 | 3.09 | 18.33 | 27.50 | 16.11 | 1.63 | 21.37 | 1.39 | 1.50 |
| lpat2-3 2 | | | | | | | | | |
| L11 | 8.20 | 3.09 | 18.04 | 27.90 | 15.18 | 1.72 | 22.84 | 1.51 | 1.53 |
| lpat2-3 3 | | | | | | | | | |
| sn-2 | | | | | | | | | |
| WT 1 | 0.78 | 0.53 | 18.54 | 54.22 | 25.93 | 0.00 | 0.00 | 0.00 | 0.00 |
| WT 2 | 0.78 | 0.52 | 17.22 | 54.11 | 27.37 | 0.00 | 0.00 | 0.00 | 0.00 |
| WT 3 | 0.75 | 0.53 | 17.38 | 53.89 | 27.44 | 0.00 | 0.00 | 0.00 | 0.00 |
| L11 1 | 9.56 | 0.68 | 14.68 | 46.54 | 28.53 | 0.00 | 0.00 | 0.00 | 0.00 |
| L11 2 | 7.83 | 0.00 | 14.05 | 46.24 | 31.88 | 0.00 | 0.00 | 0.00 | 0.00 |
| L11 3 | 8.10 | 0.00 | 14.43 | 46.01 | 31.46 | 0.00 | 0.00 | 0.00 | 0.00 |
| L11 | 9.56 | 0.37 | 15.63 | 45.83 | 28.61 | 0.00 | 0.00 | 0.00 | 0.00 |
| lpat2-2 1 | | | | | | | | | |
| L11 | 9.79 | 0.65 | 14.12 | 45.05 | 30.39 | 0.00 | 0.00 | 0.00 | 0.00 |
| lpat2-2 2 | | | | | | | | | |
| L11 | 9.29 | 0.35 | 13.62 | 49.00 | 27.74 | 0.00 | 0.00 | 0.00 | 0.00 |
| lpat2-2 3 | | | | | | | | | |
| L11 | 13.31 | 0.60 | 16.58 | 46.78 | 22.73 | 0.00 | 0.00 | 0.00 | 0.00 |
| lpat2-3 1 | | | | | | | | | |
| L11 | 13.88 | 0.95 | 15.53 | 46.81 | 22.82 | 0.00 | 0.00 | 0.00 | 0.00 |
| lpat2-3 2 | | | | | | | | | |
| L11 | 12.82 | 1.30 | 16.70 | 48.02 | 21.15 | 0.00 | 0.00 | 0.00 | 0.00 |
| lpat2-3 3 | | | | | | | | | |

qRT-PCR analysis showed that LPAT2 expression is reduced by ˜83% in developing lpat2-3 siliques, but only by ˜24% in lpat2-2. (FIG. 3B). These data support the hypothesis that LPAT2 contributes to TAG biosynthesis in Arabidopsis seeds and that it competes with ΔCTS-LPAT1. The level of C16:0 enrichment at sn-2 also appears to respond to the strength of LPAT2 repression and achieving a greater reduction than ˜83% might therefore lead to even stronger enrichment.

Disruption of PDCT Also Enhances C16:0 Incorporation Into the sn-2 Position of TAG

In developing Arabidopsis seeds >90% of the glycerol backbone in TAG is derived from the membrane lipid phosphatidylcholine (PC), owing to rapid diacylglycerol (DAG)-PC interconversion, catalysed mainly by the plant-specific head group exchange PC:DAG cholinephosphotransferase (PDCT) (FIG. 1A). Although LPAT is responsible for the initial acylation of glycerolipids at sn-2, once these acyl groups are in PC they might be removed and replaced by acyl editing activities (FIG. 1A). To determine whether bypassing glycerolipid flux through PC (FIG. 1A) might increase ΔCTS-LPAT1-dependent incorporation of C16:0 into the sn-2 position of TAG, we crossed ProGLY:ΔCTS-LPAT1 L11 into the pdct (reduced oleate desaturation1) mutant. When we purified TAG from ProGLY:ΔCTS-LPAT1 pdct seed batches and performed positional analysis, we found that the percentage of C16:0 at sn-2 had increased from ˜30% in the parental ProGLY:ΔCTS-LPAT1 line to ˜56% in the pdet background (FIG. 4A; Table 4).

TABLE 4
Total and sn-2 fatty acyl composition of TAG from
wild type (WT) and homozygous ProGLY:ΔCTS-LPAT1
seeds containing rod1 and lpat2-3 mutant alleles.

| | Fatty acid content (%) | | | | | | | | |
|---|---|---|---|---|---|---|---|---|---|
| Line | 16:0 | 18:0 | 18:1 | 18:2 | 18:3 | 20:0 | 20:1 | 20:2 | 22:1 |
| Total | | | | | | | | | |
| WT 1 | 7.84 | 3.38 | 17.20 | 28.95 | 16.89 | 2.02 | 20.2 | 1.82 | 1.69 |
| WT 2 | 7.89 | 3.43 | 17.97 | 29.09 | 16.58 | 1.90 | 19.8 | 1.69 | 1.63 |
| WT 3 | 7.65 | 3.77 | 15.95 | 28.91 | 17.43 | 2.31 | 20.5 | 1.85 | 1.59 |
| WT 4 | 7.67 | 3.80 | 17.03 | 28.57 | 16.88 | 2.18 | 20.5 | 1.74 | 1.54 |
| WT 5 | 8.14 | 3.60 | 17.39 | 28.98 | 16.12 | 2.00 | 20.4 | 1.74 | 1.61 |
| WT 6 | 7.96 | 3.35 | 15.79 | 29.27 | 17.67 | 1.89 | 20.5 | 1.73 | 1.75 |
| L11 1 | 9.06 | 3.48 | 15.98 | 28.47 | 18.30 | 0.00 | 21.4 | 1.74 | 1.51 |
| L11 2 | 6.47 | 3.08 | 15.13 | 27.52 | 19.77 | 2.18 | 22.4 | 1.52 | 1.57 |
| L11 3 | 10.16 | 3.37 | 19.48 | 27.91 | 15.94 | 1.88 | 18.6 | 1.26 | 1.33 |
| L11 4 | 7.66 | 3.15 | 38.93 | 13.00 | 13.24 | 1.72 | 20.5 | 0.39 | 1.37 |
| L11 5 | 8.35 | 3.03 | 36.89 | 14.26 | 14.30 | 1.64 | 19.7 | 0.42 | 1.41 |
| L11 6 | 10.34 | 3.27 | 18.30 | 27.89 | 16.78 | 1.85 | 18.8 | 1.37 | 1.35 |
| L11 pdct 1 | 9.63 | 3.06 | 33.88 | 13.74 | 15.46 | 1.73 | 19.3 | 1.29 | 1.82 |
| L11 pdct 2 | 8.80 | 3.15 | 34.60 | 12.20 | 14.33 | 1.86 | 20.5 | 1.64 | 2.85 |
| L11 pdct 3 | 8.75 | 2.91 | 34.46 | 14.41 | 14.33 | 1.73 | 21.1 | 0.55 | 1.70 |
| L11 lpat2-3 pdct 1 | 10.44 | 2.92 | 35.40 | 15.08 | 14.48 | 1.41 | 18.4 | 0.46 | 1.38 |
| L11 lpat2-3 pdct 2 | 10.88 | 2.84 | 35.97 | 15.31 | 13.77 | 1.27 | 18.2 | 0.53 | 1.19 |
| L11 lpat2-3 pdct 3 | 8.77 | 2.95 | 36.57 | 13.57 | 12.28 | 1.48 | 22.9 | 0.00 | 1.41 |
| L11 lpat2-3 pdct 4 | 9.36 | 2.86 | 35.16 | 13.55 | 13.69 | 1.55 | 21.5 | 0.62 | 1.60 |
| L11 lpat2-3 pdct 5 | 9.50 | 3.07 | 34.06 | 13.88 | 14.43 | 1.64 | 21.4 | 0.63 | 1.33 |
| sn-2 | | | | | | | | | |
| WT 1 | 0.67 | 0.41 | 17.41 | 53.86 | 27.65 | 0.00 | 0.00 | 0.00 | 0.00 |
| WT 2 | 0.70 | 0.52 | 17.67 | 54.01 | 27.11 | 0.00 | 0.00 | 0.00 | 0.00 |
| WT 3 | 1.48 | 1.20 | 15.30 | 52.76 | 29.26 | 0.00 | 0.00 | 0.00 | 0.00 |
| WT 4 | 0.85 | 0.85 | 16.42 | 53.22 | 28.67 | 0.00 | 0.00 | 0.00 | 0.00 |
| WT 5 | 0.80 | 0.81 | 16.33 | 54.37 | 29.78 | 0.00 | 0.00 | 0.00 | 0.00 |
| WT 6 | 0.91 | 0.86 | 14.79 | 53.66 | 28.43 | 0.00 | 0.00 | 0.00 | 0.00 |
| L11 1 | 8.54 | 0.56 | 14.09 | 48.35 | 28.46 | 0.00 | 0.00 | 0.00 | 0.00 |
| L11 2 | 6.88 | 0.33 | 12.68 | 48.70 | 31.41 | 0.00 | 0.00 | 0.00 | 0.00 |
| L11 3 | 7.90 | 0.88 | 18.17 | 48.31 | 24.74 | 0.00 | 0.00 | 0.00 | 0.00 |
| L11 4 | 6.79 | 0.39 | 47.28 | 23.74 | 21.80 | 0.00 | 0.00 | 0.00 | 0.00 |
| L11 5 | 8.00 | 0.00 | 43.04 | 25.61 | 23.36 | 0.00 | 0.00 | 0.00 | 0.00 |
| L11 6 | 8.03 | 0.79 | 17.28 | 48.36 | 25.53 | 0.00 | 0.00 | 0.00 | 0.00 |
| L11 pdct 1 | 16.16 | 0.00 | 36.12 | 23.39 | 24.32 | 0.00 | 0.00 | 0.00 | 0.00 |
| L11 pdct 2 | 16.24 | 0.59 | 38.52 | 21.43 | 23.23 | 0.00 | 0.00 | 0.00 | 0.00 |
| L11 pdct 3 | 13.35 | 0.00 | 38.81 | 25.71 | 22.14 | 0.00 | 0.00 | 0.00 | 0.00 |
| L11 lpat2-3 pdct 1 | 19.54 | 0.00 | 36.33 | 23.93 | 20.20 | 0.00 | 0.00 | 0.00 | 0.00 |
| L11 lpat2-3 pdct 2 | 22.57 | 0.38 | 35.99 | 23.04 | 18.02 | 0.00 | 0.00 | 0.00 | 0.00 |
| L11 lpat2-3 pdct 3 | 20.36 | 0.41 | 40.58 | 21.56 | 17.09 | 0.00 | 0.00 | 0.00 | 0.00 |
| L11 lpat2-3 pdct 4 | 21.18 | 0.00 | 37.97 | 21.86 | 18.99 | 0.00 | 0.00 | 0.00 | 0.00 |
| L11 lpat2-3 pdct 5 | 20.30 | 0.00 | 37.53 | 22.35 | 19.82 | 0.00 | 0.00 | 0.00 | 0.00 |

These data suggest that a more direct flux of newly made DAG into TAG (FIG. 1A) favours C16:0 incorporation and/or retention at the sn-2 position. In WT seeds it is conceivable that C16:0 entering the sn-2 position of PC might either be edited from it by the action of lysophosphatidylcholine acyltransferase (LPCAT) or a phospholipase A2. Interestingly, some groups have provided in vitro evidence that the reverse activities of Arabidopsis LPCAT1 and LPCAT2 can selectively remove certain fatty acyl groups from PC, but C16:0 was not tested.

Disruption of LPAT2 and PDCT Has an Additive Effect on Incorporation of C16:0 at sn-2

To determine whether the combination of reducing LPAT competition and bypassing flux through PC would have an additive effect on ΔCTS-LPAT1-dependent incorporation of C16:0 into the sn-2 position of TAG (FIG. 1A), we crossed ProGLY:ΔCTS-LPAT1 lpat2-3 with ProGLY:ΔCTS-LPAT1 pdct. When we purified TAG from homozygous seed batches and performed positional analysis, we found that the percentage of C16:0 at sn-2 had increased from ˜56% in ProGLY:ΔCTS-LPAT1 pdct to ˜71% in ProGLY:ΔCTS-LPAT1 lpat2-3 pdct (FIG. 4A; Table 4). The combination of just three modifications to the TAG biosynthetic pathway in Arabidopsis (i.e. ΔCTS-LPAT1 expression, plus LPAT2 and PDCT suppression) is therefore sufficient to replicate the level of C16:0 enrichment at the sn-2 position (versus sn-1+3) that is found in HMF. Analysis of TAG composition in ProGLY:ΔCTS-LPAT1 lpat2-3 pdct (All) seeds using high resolution/accurate mass (HR/AM) lipidomics also confirmed the presence of C16:0 groups at the sn-2 position, since tripalmitin was 27-fold more abundant than in WT (FIG. 6A). By contrast, no dipalmitoyl PC was detected in ProGLY:ΔCTS-LPAT1 lpat2-3 pdct seeds and molecular species of PC containing one C16:0 group were not increased (FIG. 6B). These data suggest that an asymmetrical distribution of saturated and unsaturated fatty acyl groups in PC is maintained in ProGLY:ΔCTS-LPAT1 lpat2-3 pct seeds and this may be important to prevent membranes assuming the gel phase at physiological temperatures.

### Redistribution of C16:0 Reduces Seed Oil Content, but not Germination or Establishment

Many studies have shown that modifying fatty acyl composition can reduce TAG accumulation in oilseeds and in some cases can also impair seed germination and seedling establishment. Our primary objective in this study was not to alter fatty acyl composition per se, but to change the stereoisomeric structure of TAG. To examine the physiological impact of C16:0 enrichment at the sn-2 position of TAG, we compared seed batches from wild type and ProGLY:ΔCTS-LPAT1 lpat2-3 pdct plants that had been grown together under standard laboratory conditions. We found no significant difference (P>0.05) in seed weight between the two genotypes (FIG. 4B). However, the fatty acid content of ProGLY:ΔCTS-LPAT1 lpat2-3 pdct seeds was significantly (P<0.05) lower than that of wild type, when expressed as a percentage of seed weight (FIG. 4C). These data suggest that the modifications leading to incorporation of C16:0 into the sn-2 position, reduce TAG biosynthetic flux. This finding is consistent with previous studies in which seed TAG composition has been modified either using genetic engineering or mutant breeding methods. Despite the reduction in fatty acid content, ProGLY:ΔCTS-LPAT1 lpat2-3 pdct seed germination, scored as radicle emergence (FIG. 5A) and seedling establishment, scored as cotyledon expansion (FIG. 5B) and true leaf development (FIG. 5C), did not appear to be significantly (P<0.05) impaired. TAG breakdown also was not impeded in ProGLY:ΔCTS-LPAT1 lpat2-3 pdct seeds following germination (FIG. 5D), and this contrasts with some studies where seeds have been modified to incorporate uncommon fatty acyl groups into TAG. Finally, although ProGLY:ΔCTS-LPAT1 lpat2-3 pdct carries a hypomorphic allele of the essential gene LP AT2, this does not appear to adversely affect growth and morphology at the rosette stage (FIG. 7).

### Materials and Methods

### Plant Material and Growth Conditions

Arabidopsis thaliana wild type (Col-0) and LPAT2 (At3g57650) T-DNA insertion lines SALK_108026 (lpat2-2) and SALK_004681 (lpat2-3) (1) were obtained from the European Arabidopsis Stock Centre (University of Nottingham, UK). The pdct (rod1) mutant has been described previously (2). Seeds were surface sterilized, stratified at 4° C. for two days and germinated on agar plates containing ½ strength MS medium (Sigma-Aldrich) pH 5.7. Seedlings were transplanted to 7 cm² pots containing Levington F2 compost and grown in a chamber set to a 16-h light (22° C.)/8-h dark (16° C.) cycle, with a light intensity of 250 μmol m⁻² s⁻¹. The plants were bagged individually at the onset of flowering and the seeds were harvested at maturity.

### Cloning and Transformation

RNA was isolated from Brassica napus (cv. Kumily) seedlings and DNAse treated using the RNAeasy Plant Mini kit (Qiagen). RNA was reverse transcribed into cDNA with the SuperScript III Reverse Transcriptase kit (ThermoFisher Scientific). B. napus LPAT1 (GenBank: AF111161) lacking the chloroplast targeting sequence (CTS) was amplified by PCR with KOD DNA polymerase (Merck) using primer pair P1+P2 (Table 5).

TABLE 5
Primers used in study

| | |
|---|---|
| P1: | 5′-CACCATGTCGGATCTTTCAGGAGCTG-3′ |
| | (SEQ ID NO: 3) |
| P2 | 5′-TCAACTCAAGAGATTCATAGATTCA-3′ |
| | (SEQ ID NO: 4) |
| P3 | 5′-CGGAATTCATGTCGGATCTTTCAGGAGC-3′ |
| | (SEQ ID NO: 5) |
| P4 | 5′-GCTCTAGATCAACTCAAGAGATTCATAGATTC-3′ |
| | (SEQ ID NO: 6) |
| P5: | 5′-GTTGATTGAATCGAGGAAGGA-3′ |
| | (SEQ ID NO: 7) |
| P6: | 5′-CTTTTTACCACATGCAAAGGG-3′ |
| | (SEQ ID NO: 8) |
| P7: | 5′-CCATTTTGGTGGTCGTCTAT-3′ |
| | (SEQ ID NO: 9) |
| P8: | 5′-CAGAAAAATTAACCGGGTGGT-3′ |
| | (SEQ ID NO: 10) |
| P9: | 5′-CAGCTGAAACCGACGTCTCT-3′ |
| | (SEQ ID NO: 11) |
| P10: | 5′-GGTCACGCGCTCTTTGAATC-3′ |
| | (SEQ ID NO: 12) |
| P11: | 5′-GTAGCACCCAAGGCTTCCA-3′ |
| | (SEQ ID NO: 13) |
| P12: | 5′-GCCAGTGATCATCAGGACG-3′ |
| | (SEQ ID NO: 14) |
| P13: | 5′-GCGTGGACCGCTTGCTGCAACT-3′ |
| | (SEQ ID NO: 15) |
| P14: | 5′-CTGGTGGGCTGGAGTTAAGA-3′ |
| | (SEQ ID NO: 16) |
| P15: | 5′-AGAATCCATCCCACAAGCCA-3′ |
| | (SEQ ID NO: 17) |
| P16: | 5′-TGTCATCGGTTGGGCTATGT-3′ |
| | (SEQ ID NO: 18) |
| P17: | 5′-CATCAGCGTTATTGGCACCA-3′ |
| | (SEQ ID NO: 19) |
| P18: | 5′-GACGCTTCATCTCGTCC-3′ |
| | (SEQ ID NO: 20) |
| P19: | 5′-CCACAGGTTGCGTTAG-3′ |
| | (SEQ ID NO: 21) |
| P20: | 5′-TCCAGCTAAGGGTGCC-3′ |
| | (SEQ ID NO: 22) |
| P21: | 5′-GGTGGGTACTCGGAGA-3′ |
| | (SEQ ID NO: 23) |
| P22: | 5′-GAATTACCCGACGGACA-3′ |
| | (SEQ ID NO: 24) |
| P23: | 5′-ACGGTCTGCAATACCT-3′ |
| | (SEQ ID NO: 25) |

The resulting PCR product was purified with the QIAquick Gel Extraction Kit (Qiagen). For localisation studies, ΔCTS-LPAT1 was cloned behind Red Fluorescent Protein (RFP) in the pK7WGR2 vector (Vlaams Institute for Biotechnology). The PCR product was cloned in the pENTR™/D-TOPO™ vector (Thermo Fisher Scientific), sequenced (FIG. 1B) and recombined into the pK7WGR2 vector using the Gateway™ LR Clonase™ II Enzyme mix (Thermo Fisher Scientific). ΔCTS-LPAT1 was cloned in the pBinGlyRed3 vector in between the soybean glycinin-1 (GLY) promoter and terminator for seed specific expression (3). ΔCTS-LPAT1 was PCR-amplified from the pENTR-D-TOPO vector using KOD DNA polymerase and primer pair P3+P4. The PCR product was gel purified and digested with EcoRI and XbaI. The pBinGly Red3 vector was also digested with EcoRI and XbaI, alkaline phosphatase treated (Promega), gel purified and ΔCTS-LPAT1 was ligated into the vector using T4 DNA ligase (NEB). Heat shock was used to transform the vectors into Agrobacterium tumefaciens strain GV3101 and Arabidopsis transformation was then carried out using the floral-dip method (4). T1 seeds expressing the selectable marker were identified under a Leica M205 FA microscope using the DsRed filter.

Transient Expression in Nicotiana benthamiana and Imaging

Transient expression in Nicotiana benthamiana leaves was carried out as described by Wood et al., (5) using A. tumefaciens cultures transformed with vectors harbouring Pro35S: RFP-ΔCTS-LPAT1, Pro35S: m-GFP5-ER or Pro35S: p19. Cultures were hand-infiltrated into leaves and the inoculated plants were left for 48 h. N. benthamiana leaves were then mounted in water on a Zeiss LSM 780 laser scanning confocal microscope under an Apochromat 63×/1.20 W Korr M27 objective. GFP was excited at a wavelength of 488 nm and RFP at 561 nm. Filters with an emission band at 473-551 nm were used for detection.

### Genotyping

Genomic DNA was isolated using the DNeasy Plant Mini Kit (Qiagen). Homozygous T-DNA insertional mutants were identified by PCR (1) using the Promega PCR Master Mix (Promega). The following primer pairs were used for genotyping lpat2-2 (WT: P5+P6 or T-DNA: P5+P7) and lpat2-3 (WT: P8+P9 or T-DNA: P8+P7). PDCT was genotyped by sequencing PCR products amplified with primer pair P10+P11 spanning the site of the point mutation (2). ProGLY:ΔCTS-LPAT1 was genotyped by PCR using primer pair P12+P13 spanning ProGLY and ΔCTS-LPAT1.

### Lipid Analysis

Total lipids were extracted from seeds and seedlings and TAG was purified as described previously (6). TAG stereochemical analysis was performed by lipase digestion following the method described previously (7), except that 2-monoacylglycerols were separated by thin layer chromatography (Silica gel 60, 20×20 cm; Sigma-Aldrich/Merck) using hexane: diethylether: acetic acid (35:70:1.5, v/v/v) (8). Fatty acyl groups present in whole seeds and purified lipid fractions were trans-methylated and quantified by gas chromatography (GC) coupled to flame ionization detection, as described previously (9), using a 7890A GC system fitted with DB-23 columns (30 m×0.25 mm i.d.×0.25 μm) (Agilent Technologies).

TAG and PC molecular species composition were analysed by high resolution/accurate mass (HR/AM) lipidomics (10-12) using a Vanquish—Q Exactive Plus UPLC-MS/MS system (Thermo Fisher Scientific). Work flow consisted of using total lipids purified at 3 μg/μl and diluted 1 in 100 in chloroform: methanol (1:1, v/v). Internal tripalmitin standard (0.857 μM) was added and 20 μl injected into the UPLC. Lipids were separated using a Accucore C18 (2.1×150 mm, 2.6 mm) column (Thermo Fisher Scientific) at 35° C. with autosampler tray temperature, 10° C. flow rate at 400 μl min⁻¹. Mobile phase: A=10 mM ammonium formate in 50% acetonitrile+0.1% formic acid, B=2 mM ammonium formate in acetonitrile:propan-2-ol:water (10:88:2 v/v)+0.02% formic acid. Elution gradient ran for 28 minutes from 35% B at start to 100% at 24 mins. Thermo Q Exactive HESI II probe conditions, sweep plate in use probe position in C. Conditions were adjusted for separate positive and negative runs, running samples in a single polarity resulted in more identifications. LC/MS at 140K resolution and data-independent HCD MS2 experiments (35K resolution) were performed in positive and negative ion modes. Full Scan @ 140,000 resolution m/z 150-1200 Top 15 most abundant MS/MS @ 35,000 resolution using an isolation window of 1 m/z, maximum integration time of 75 ms and dynamic exclusion window of 8s. The stepped collision energy was 25, 30, 40 eV replacing 25 with 30 eV negative ion mode. Sheath gas set to 60, Aux gas 20, sweep gas 1 spray voltage 3.2 KV in positive ion mode with small adjustments in negative ion mode, capillary temperature 320 and aux gas heater set to 370° C. LipidSearch 4.2 experimental workflow (Thermo Fisher Scientific) was used for lipid characterization and potential lipid species were identified separately from positive or negative ion adducts. The data for each biological replicate were aligned within a chromatographic time window by combining the positive and negative ion annotations and merging these into a single lipid annotation.

qRT-PCR Analysis

DNAse-treated total RNA was isolated from developing siliques as described by Mendes et al., (13). The synthesis of single stranded cDNA was carried out using SuperScript™II RNase H-reverse transcriptase from Invitrogen Ltd. (Paisley, UK). Quantitative (q)-PCR was performed as described previously (13), except that LPAT2 and ΔCTS-LPAT1 expression were normalized to the geometric mean of three reference genes (UBQ5, EF-1a and ACT8) that were selected owing to their stable expression over the course of seed development (14). Primer pairs P14+P15, P16+P17, P18+P19, P20+P21 and P22+P23, were used for LPAT2, ΔCTS-LPAT1, UBQ5, EF-1a and ACT8, respectively.

### Seed Germination and Establishment Assays

Around 50 seeds from each plant were sown on a ½MS agar plate, stratified at 4° C. for two days and transferred to a growth chamber set to 200C, 16 h light/8 h dark, PPFD=150 μmol m⁻² s⁻¹. Germination (radicle emergence), expanded cotyledons and expanded true leaves were scored visually under a dissecting stereomicroscope every day for a week. Seeds and seedlings were also collected at zero and four days after stratification for lipid analysis.

### Statistical Analysis

All experiments were carried out using between three and six biological replicates and the data are presented as the mean values±standard error of the mean (SE). For statistical analysis we either used one-way analysis of variance (ANOVA) with post-hoc Tukey HSD (Honestly Significant Difference) tests, or two-tailed Student's t-tests.

### Conclusions

As described herein it is shown that the TAG biosynthetic pathway in plants can be engineered so that the stereoisomeric structure of seed storage oil is altered to mimic that of HMF, with >70% of C16:0 concentrated at the middle (sn-2 or B) position on the glycerol backbone. There is mounting evidence that this configuration is beneficial for infant nutrition, but it has not been found to occur naturally in vegetable fats where C16:0 is virtually excluded from the sn-2 position. Many infant formulas contain HMFS that are made by restructuring vegetable fats using enzyme-based catalysis, but they are relatively costly to produce; particularly for the manufacture of true mimetics with >70% of C16:0 at the sn-2 position. Translation of our technology from the model species Arabidopsis to an oilseed crop might conceivably provide a cheaper and more sustainable source of HMFS for infant formula, since it would abrogate the need for enzyme-based catalysis. The infant formula market is currently estimated to use around 450,000 metric tons of vegetable-derived fat per year, of which about 38,000 metric tons is HMFS. Several oilseed crops may be considered as possible hosts for HMFS production, and it is noteworthy that conventional sunflower and genetically modified oilseed rape varieties have already been developed that have the appropriate fatty acyl composition. Even an oilseed crop with more modest C16:0 enrichment at the sn-2 position that we have achieved here may still be desirable since clinical trials have reported benefits with as little as 43% of C16:0 at the sn-2 position and product surveys have found that this level of enrichment is common in infant formulas that are supplemented with HMFS.

Further studies have been conducted as detailed below.

### Example 2

fab1-1 fae1 Background

We crossed the ProGLY:ΔCTS-LPAT1 lpat2-3 pct line described in Example 1 into the fab1-1 fae1 mutant (15) to create ProGLY:ΔCTS-LPAT1 lpat2-3 pct fab 1-1 fae1. This Arabidopsis thaliana background has elevated levels of C16:0 in its seed TAG owing to a hypomorphic allele of the FAB1 gene encoding chloroplast 3-keto-acyl-ACP synthase II (KASII), and also reduced very long chain fatty acid levels due to a null FATTY ACID ELONGASE 1 (FAE1) allele (Nguyen et al., 2010). When we performed positional analysis, we found that the percentage of C16:0 at sn-2 was ˜2% in fab1-1 fae1 and ˜60% in ProGLY:ΔCTS-LPAT1 lpat2-3 pdct fab1-1 fae1 (Table 6). The data show that C16:0 enrichment at the sn-2 position in TAG can be produced in seeds with higher levels of total C16:0 than are present in WT seeds.

TABLE 6
Total and sn-2 C16:0 content of TAG from A. thaliana fab1-1 fae1 and
ProGLY:ΔCTS-LPAT1 lpat2-3 pdct fab1-1 fae1 seeds. Values are
measurements made on separate seed batches
from two plants of each genotype.

| Genotype | Total C16:0 (%) | % of C16:0 at sn-2 |
|---|---|---|
| fab1-1 fae1 | 19.31 | 2.11 |
| fab1-1 fae1 | 18.60 | 3.03 |
| ProGLY:ΔCTS-LPAT1 | 20.21 | 62.29 |
| lpat2-3 pdct fab-1 fae1 | | |
| ProGLY:ΔCTS-LPAT1 | 19.06 | 57.80 |
| lpat2-3 pdct fab-1 fae1 | | |

### Example 3

### Human LPAT AGPAT1

We expressed a plant codon optimised version of the human LPAT AGPAT1 under the control of the glycinin promoter in wild type (WT) A. thaliana Col-0 and fab1-1 fae1 seeds following the procedures described in Example 1. When we performed positional analysis, we found that the percentage of C16:0 at sn-2 was up to ˜73% in WT and up to ˜55% in fab1-1 fae1 background (Table 7). The data show that C16:0 enrichment at the sn-2 position in TAG can be produced in seeds by expression of AGPAT1.

TABLE 7
Total and sn-2 C16:0 content of TAG from A. thaliana WT, fab1-1
fae1, ProGLY:AGPAT1 and ProGLY:AGPAT1 L3
fab1-1 fae1 seeds. Values are means ± SE of measurements
made on separate seed batches from three plants of each genotype.

| Genotype | Total C16:0 (%) | % of C16:0 at sn-2 |
|---|---|---|
| WT | 8.22 ± 0.13 | 1.97 ± 0.31 |
| ProGLY:AGPAT1 L35 | 7.19 ± 0.16 | 73.35 ± 1.99 |
| ProGLY:AGPAT1 L40 | 7.77 ± 0.22 | 69.40 ± 1.46 |
| fab1-1 fae1 | 18.35 ± 0.31 | 2.69 ± 1.12 |
| ProGLY:AGPAT1 L3 fab1-1 fae1 | 15.49 ± 0.60 | 54.77 ± 1.22 |
| ProGLY:AGPAT1 L9 fab1-1 fae1 | 15.41 ± 2.11 | 43.55 ± 1.01 |

Codon optimized AGPATI DNA sequence
(SEQ ID NO: 26)
ATGGATTTATGGCCTGGTGCTTGGATGTTATTATTATTATTATTCTTGTT
ATTGCTCTTCCTCCTTCCTACTTTGTGGTTCTGTTCACCTTCTGCAAAGT
ATTTCTTTAAGATGGCTTTCTACAACGGATGGATTCTTTTCTTGGCTGTT
TTGGCAATCCCAGTTTGTGCTGTGAGAGGTAGGAACGTTGAAAACATGAA
GATTCTCAGACTCATGCTTTTGCATATCAAGTACCTTTACGGAATAAGAG
TGGAAGTTAGGGGTGCTCATCACTTTCCTCCATCTCAACCTTATGTTGTG
GTTTCAAACCACCAGTCTTCATTGGATCTCTTAGGAATGATGGAAGTGCT
CCCTGGTAGATGTGTTCCAATAGCAAAGAGGGAGCTTTTGTGGGCTGGAT
CAGCAGGTCTTGCTTGCTGGTTGGCTGGAGTTATTTTTATCGATAGAAAA
AGGACAGGTGATGCAATCTCAGTGATGAGTGAAGTTGCTCAAACTCTCTT
AACACAGGATGTGAGAGTTTGGGTGTTCCCTGAGGGAACCAGGAATCATA
ACGGTAGTATGTTACCATTTAAGAGAGGAGCTTTCCACCTCGCAGTTCAA
GCTCAGGTTCCTATAGTGCCAATAGTTATGAGTTCTTACCAAGATTTCTA
CTGTAAGAAGGAAAGAAGGTTCACTAGTGGACAATGCCAAGTTAGAGTGC
TCCCTCCAGTTCCAACCGAGGGTTTAACTCCTGATGATGTGCCAGCTCTT
GCAGATAGAGTTAGGCATTCTATGTTGACAGTGTTCAGAGAGATTAGTAC
CGATGGAAGGGGAGGTGGAGATTATTTGAAGAAGCCAGGTGGAGGAGGAT
GA

### Example 4

Camelina sativa

We expressed ΔCTS-LPAT1 in Camelina sativa while simultaneously supressing endogenous ER LPAT. We constructed a multigene T-DNA vectors in the pBinGlyRed3 backbone (3) containing ΔCTS-LPAT1 and LPAT2-like gene specific artificial microRNAs (amiRNAs) under the control of the seed-specific oleosin and napin promoters, respectively. Three amiRNA sequences were selected to target C. sativa LPAT2-like genes (5′-TAAAGCGAGTTCCCTCGACAG-3′ (SEQ ID NO:27), 5′-TTGTGCCCAGTGTACGGACTT-3′ (SEQ ID NO:28) and 5′-TCAAAGGCACGATGATACCTG-3′ (SEQ ID NO:29)) and used to replace the stem loops in the Arabidopsis thaliana MIR319a precursor (16). The constructs were transformed into C. sativa cv Suneson (WT) using Agrobacterium tumefaciens (17). Homozygous T3 seeds batches were obtained for multiple independent lines and lipid analysis was performed as described in Example 1. When we performed positional analysis, we found that the percentage of C16:0 at sn-2 was ˜1% in WT and ˜70% in ProOLE: ΔCTS-LPAT1 ProNAP:LPAT2amiRNA seeds (Table 8). The data show that C16:0 enrichment at the sn-2 position in TAG can be produced in C. sativa seeds.

TABLE 8
Total and sn-2 C16:0 content of TAG from C. sativa WT and
ProOLE:ΔCTS-LPAT1 ProNAP:LPAT2amiRNA seeds. Values are
from measurements made on single seed batches of each genotype.

| | Total | C16:0 % of |
|---|---|---|
| Genotype | (%) | C16:0 at sn-2 |
| WT (cv Suneson) | 4.39 | 0.77 |
| ProOLE:ΔCTS-LPAT1 ProNAP: | 5.28 | 66.46 |
| LPAT2amiRNA L11 | | |
| ProOLE:ΔCTS-LPAT1 ProNAP: | 5.62 | 76.57 |
| LPAT2amiRNA L12 | | |

### Example 5

Brassica napus

We expressed ΔCTS-LPAT1 or AGPAT1 in Brassica napus seeds while simultaneously supressing endogenous ER LPAT and also expressing Arabidopsis thaliana FATB to increase total C16:0 content. We constructed multigene T-DNA vectors in the pBinGlyBar1 backbone (3) containing FATB, LPAT2-like gene specific amiRNAs and ΔCTS-LPAT1 or AGPAT1 under the control of the seed-specific gene promoters oleosin, napin and glycinin, respectively. We selected three amiRNA sequences to target B. napus LPAT2-like genes (5′-TCACTTGATGTGAAGATGCAC-3′ (SEQ ID NO:30), 5-'TTAACAGCTGACACGAAGCCT-3′ (SEQ ID NO:31) and 5′-TCACTTGATGTGAACACGCAC-3′ (SEQ ID NO:32)) and used them to replace the stem loops in the Arabidopsis thaliana MIR319a precursor (16). The constructs were transformed into Brassica napus cv DH12075 (WT) using Agrobacterium tumefaciens (18). Homozygous T3 seeds batches were obtained for multiple independent lines and lipid analysis was performed as described in Example 1. When we performed positional analysis, we found that the percentage of C16:0 at sn-2 was ˜2% in WT and up to 47% and 62% in ProOLE:FATB ProNAP:LPATamiRNA ProGLY:ΔCTS-LPAT1 and ProOLE:FATB ProNAP:LPATamiRNA ProGLY:AGPAT seeds, respectively (Table 9). Total C16:0 content in seeds of the transgenic lines was also increased to between 24 and 30%. The data show that C16:0 enrichment at the sn-2 position in TAG can be produced in B. napus seeds that are also modified to have enhanced total C16:0 content.

FATB DNA sequence (Arabidopsis thaliana)
(SEQ ID NO: 33)
ATGGTGGCCACCTCTGCTACGTCGTCATTCTTTCCTGTACCATCTTCTTC
ACTTGATCCTAATGGAAAAGGCAATAAGATTGGGTCTACGAATCTTGCTG
GACTCAATTCTGCACCTAACTCTGGTAGGATGAAGGTTAAACCAAACGCT
CAGGCTCCACCTAAGATTAATGGGAAAAAGGTTGGTTTGCCTGGTTCTGT
AGATATTGTAAGGACTGATACCGAGACCTCATCACACCCTGCGCCGAGAA
CTTTCATCAACCAGTTACCTGACTGGAGCATGCTTCTTGCTGCTATAACT
ACGATTTTCTTAGCGGCTGAGAAACAGTGGATGATGCTTGATTGGAAACC
TAGGCGTTCTGACATGCTGGTGGATCCTTTTGGTATAGGGAGAATTGTTC
AGGATGGCCTTGTGTTCCGTCAGAATTTTTCTATTAGGTCATATGAAATA
GGTGCTGATCGCTCTGCATCTATAGAAACCGTCATGAATCATCTGCAGGA
AACGGCGCTTAATCATGTTAAGACTGCTGGATTGCTTGGAGATGGGTTTG
GCTCTACACCTGAGATGTTTAAGAAGAACTTGATATGGGTTGTCACTCGT
ATGCAGGTTGTGGTTGATAAATATCCTACTTGGGGAGATGTTGTTGAAGT
AGACACCTGGGTCAGTCAGTCTGGAAAGAATGGTATGCGTCGTGATTGGC
TAGTTCGGGACTGTAATACTGGAGAAACCTTAACACGAGCATCAAGTGTG
TGGGTGATGATGAATAAACTGACAAGGAGATTGTCAAAGATTCCTGAAGA
GGTTCGAGGGGAAATAGAGCCTTATTTTGTGAATTCTGATCCTGTCCTTG
CCGAGGACAGCAGAAAGTTAACAAAAATTGATGACAAGACTGCTGACTAT
GTTCGATCTGGTCTCACTCCTCGATGGAGTGACCTAGATGTTAACCAGCA
TGTGAATAATGTAAAGTACATTGGGTGGATCCTGGAGAGTGCTCCAGTGG
GAATAATGGAGAGGCAGAAGCTGAAAAGCATGACTCTGGAGTATCGGAGG
GAATGCGGGAGAGACAGTGTGCTTCAGTCCCTCACTGCAGTTACGGGTTG
CGATATCGGTAACCTGGCAACAGCGGGGGATGTGGAATGTCAGCATTTGC
TCCGACTCCAGGATGGAGCGGAAGTGGTGAGAGGAAGAACAGAGTGGAGT
AGTAAAACACCAACAACAACTTGGGGAACTGCACCGTAA
FATB amino acid sequence (Arabidopsis thaliana)
(SEQ ID NO: 73)
MVATSATSSFFPVPSSSLDPNGKGNKIGSTNLAGLNSAPNSGRMKVKPNA
QAPPKINGKKVGLPGSVDIVRTDTETSSHPAPRTFINQLPDWSMLLAAIT
TIFLAAEKQWMMLDWKPRRSDMLVDPFGIGRIVQDGLVFRQNFSIRSYEI
GADRSASIETVMNHLQETALNHVKTAGLLGDGFGSTPEMFKKNLIWVVTR
MQVVVDKYPTWGDVVEVDTWVSQSGKNGMRRDWLVRDCNTGETLTRASSV
WVMMNKLTRRLSKIPEEVRGEIEPYFVNSDPVLAEDSRKLTKIDDKTADY
VRSGLTPRWSDLDVNQHVNNVKYIGWILESAPVGIMERQKLKSMTLEYRR
ECGRDSVLQSLTAVTGCDIGNLATAGDVECQHLLRLQDGAEVVRGRTEWS
SKTPTTTWGTAP

TABLE 9
Total and sn-2 C16:0 content of TAG from B. napus WT, ProOLE:FATB
ProNAP:LPATamiRNA ProGLY: ΔCTS-LPAT1 and ProOLE:FATB
ProNAP:LPATamiRNA ProGLY:AGPAT1 seeds. Values are means ±
SE of measurements made on separate seed batches from three plants of
each genotype.

| Genotype | Total C16:0 | % of C16:0 as sn-2 |
|---|---|---|
| WT (DH12075) | 4.3 ± 0.1 | 2.3 ± 0.3 |
| ProOLE:FATB ProNAP:LPATamiRNA | 24.3 ± 0.9 | 47.2 ± 1.99 |
| ProGLY: ΔCTS-LPAT1 L1 | | |
| ProOLE:FATB ProNAP:LPATamiRNA | 29.8 ± 2.2 | 44.9 ± 1.46 |
| ProGLY: ΔCTS-LPAT1 L2 | | |
| ProOLE:FATB ProNAP:LPATamiRNA | 24.1 ± 0.8 | 61.8 ± 1.12 |
| ProGLY:AGPAT1 L1 | | |
| ProOLE:FATB ProNAP:LP ATamiRNA | 25.9 ± 1.4 | 59.77 ± 1.22 |
| ProGLY:AGPAT1 L2 | | |

### Example 6

Yarrowia lipolytica

We overexpressed ΔCTS-LPAT1 (PLPAT, SEQ ID NO:38), AGPAT1 (SEQ ID NO:37) or a Chlamydomonas reinhardtii LPAT (CRELPAT, SEQ ID NO:36) in the oleaginous yeast Yarrowia lipolytica. When we performed TAG positional analysis, we found that the percentage of C16:0 at sn-2 was ˜3% in WT and increased to up to ˜63% in some mutant strains (Table 14). The data show that C16:0 enrichment at the sn-2 position in TAG can be produced in Y. lipolytica cells.

The E. coli and yeast strains used in this study are listed in Table 10 and Table 11, respectively, and their culture maintenance and growth conditions have been described by Sambrook and Russell (2001) (20) and Barth and Gaillardin (1996) (19), respectively. Y. lipolytica media, culture growth conditions and biomass harvesting for lipid extraction under nitrogen limitation were the same as reported previously (Bhutada et al., 2017) (23). For the growth of ura3Δ or leu2Δ auxotrophic strains media were supplemented with 0.1 g L⁻¹ uracil or leucine.

All PCR reactions for cloning and amplification of sequencing templates were performed using Herculase II Fusion DNA Polymerase (Agilent Technologies), and with GoTaq (Promega) for confirmation of chromosomal integration of the transformation cassettes. The restriction enzymes used in this study were obtained from Roche or New England Biolabs (NEB). The DNA fragments from PCR and restriction digestion were recovered from agarose gels using GeneJET kits (Thermo Scientific). For ligations, the Fast-Link DNA Ligation Kit (Epicenter) or Gibson assembly (Gibson et al., 2009 (21); Kulasekara, 2011 (22)) was used. For transformation into Y. lipolytica standard protocols for lithium acetate were used (Le Dall et al., 1994 (25)). All primers are listed in Table 12.

TABLE 10
Plasmids used in this study.

| Plasmid | Genotype | Reference |
|---|---|---|
| pFA6aURA3-09 | Parent Plasmid (5′_MCS-loxP- | (Bhutada |
| | PPURA3TT-loxP-MCS_3′) | et al., 2017) |
| pGMKGSY12 | YlGSY1^{P}-loxP-PURA3T- | (Bhutada |
| | loxP-YlGSY1^{T} | et al., 2017) |
| YlAGPAT1 | pUC57-5′_AGPAT1-Syn^{T}_3′ | This work/ |
| | | GenScript |
| YlCRELPAT | pUC57-5′_CRELPAT-Syn^{T}_3′ | This work/ |
| | | GenScript |
| YlPLPAT | pUC57-5′_PLPAT-Syn^{T}_3′ | This work/ |
| | | GenScript |
| pGSYTEF | YlGSY1^{P}-TEF1^{P}-loxP-PURA3T- | This work |
| | loxP-YlGSY1^{T} | |
| pTEFAGPAT1 | YlGSY1^{P}-TEF1^{P}-AGPAT1-Syn^{T}- | This work |
| | loxP-PURA3T-loxP-YlGSY1^{T} | |
| pTEFCRELPAT | YlGSY1^{P}-TEF1^{P}-CRELPAT-Syn^{T}- | This work |
| | loxP-PURA3T-loxP-YlGSY1^{T} | |
| pTEFPLPAT | YlGSY1^{P}-TEF1^{P}-PLPAT-Syn^{T}-loxP- | This work |
| | PURA3T-loxP-YlGSY1^{T} | |

TABLE 11
Strains used in this study.

| strains | Genotype | source |
|---|---|---|
| Yarrowia | | |
| lipolytica | | |
| WT (W29) | MATa wild type | (Barth and |
| | | Gaillardin, |
| | | 1996) |
| PO1d | MATa ura3-302, xpr2-322, pURA3-SUC2 | (Barth and |
| (JMY2101) | | Gaillardin, |
| | | 1996) |
| gsy1Δ | MATa ura3-302, xpr2-322, pURA3-SUC2, | This work |
| | gsy1Δ::URA3 | |
| gsy1Δ- | MATa ura3-302, xpr2-322, pURA3-SUC2, | This work |
| AGPAT1 | gsy1Δ::YlGSY1^{P}-AGPAT1-Syn^{T}-URA3 | |
| gsy1Δ- | MATa ura3-302, xpr2-322, pURA3-SUC2, | This work |
| CreLPAT | gsy1Δ::YlGSY1^{P}-CreLPAT-Syn^{T}-URA3 | |
| gsy1Δ- | MATa ura3-302, xpr2-322, pURA3-SUC2, | This work |
| PLPAT | gsy1Δ::YlGSY1^{P}-PLPAT-Syn^{T}-URA3 | |

TABLE 12
Primers used in this study

| Primer | Sequence 5′ to 3′ |
|---|---|
| TEF- | CTCGCAACAACCGATTCCAACAAGAGACCGGGTTGGCG |
| GSY-F | GCGCA (SEQ ID NO: 34) |
| TEF- | ATAACTTCGTATAATGTATGCTATACGAAGTTATAAGC |
| GSY-R | TTTGAATGATTCTTATACTCAGAAGGAAATGCTTAA |
| | (SEQ ID NO: 35) |

### Construction of a Mutant With Lysophosphatidic Acid Acyltransferase (LPAT) Expression

To obtain a strain with LPAT expression, gene synthesis of codon optimized LPAT CDS sequence belonging to Human, Plant and Algal species together with Tsynt25 (SynT) synthetic terminator fragment (Curran et al., 2015) was done. The previously described plasmid pGMKGSY_12 (Bhutada et al., 2017) harbouring glycogen storage elimination cassette flanked by 1 kb recombination regions for the glycogen synthase, GSY1 locus was linearized with HindIII digest and for strong constitutive expression of LPAT genes assembled by Gibson assembly with the TEF1 promoter fragment, which was PCR amplified from W29 genomic DNA with the primers TEF-GSY-F/TEF-GSY-R resulting in plasmid pGSYTEF.

The gene synthesized pUC7 vectors harbouring LPAT CDS-SynT was digested with HindIII to excise the cloning inserts corresponding to AGPAT, CRELPAT and PLPAT. pGSYTEF was digested with the same enzyme to linearize the vector and it was re-ligated with the above gel purified inserts under the TEF1P promoter fragment, resulting in pTEFAGPAT1, pTEFCRELPAT and pTEFPLPAT. The correct assembly of the episomal YlGSY1P-loxP-URA3-loxP-TEF^{P}AGPAT1Syn^{T}-YlGSY1^{T}, YlGSY1^{P}-loxP-URA3-loxP-TEF^{P}CRELPATSyn^{T}-YlGSY1^{T}, and YlGSY1^{P}-loxP-URA3-loxP-TEF^{P}PLPATSyn^{T}-YlGSY1^{T} cassette was confirmed by sequencing. These cassettes were excised NotI digested, purified and used for transformation of strain PO1d (Table 11). Transformants with integration of the cassette at the GSY1 locus were identified by Lugol's iodine staining (1% KI, 0.5% I₂) and confirmed by control primer PCR and sequencing.

### Analytical Methods

Lipids were extracted and analysed as described in Example 1.

TABLE 13
Total and sn-2 fatty acyl composition of TAG of Y. lipolytica strains in
nitrogen-limited media. Values are means ± SD of measurements
made on three separate cultures for each genotype.

| Strain | 16:0 | 18:0 | 16:1 | 18:1 | 18:2 |
|---|---|---|---|---|---|
| | Total fatty acid composition in TAG (%) | | | | |
| WT | 21.28 ± 1.21 | 11.7 ± 0.45 | 6.49 ± 0.48 | 46.31 ± 2.4 | 14.22 ± 1.22 |
| PO1d | 21.07 | 9.4 | 6.91 | 42.9 | 19.73 |
| gsy1Δ | 9.68 ± 0.89 | 11.55 ± 0.78 | 4.68 ± 0.57 | 57.96 ± 2.16 | 16.13 ± 1.37 |
| gsy1Δ-AGPAT1 | 16.56 ± 0.6 | 11.28 ± 0.3 | 3.65 ± 0.31 | 52.8 ± 0.85 | 15.7 ± 0.54 |
| gsy1Δ-CreLPAT | 19.19 ± 1.31 | 12.64 ± 4.13 | 3.33 ± 0.21 | 50.26 ± 4.18 | 14.58 ± 1.78 |
| gsy1Δ-PLPAT | 8.98 ± 0.22 | 14.17 ± 0.12 | 3.67 ± 0.05 | 60.46 ± 0.47 | 12.72 ± 0.23 |
| | Fatty acid species at sn-2 position (%) | | | | |
| WT | 2.21 ± 1.29 | 1.9 ± 1.87 | 2.06 ± 0.25 | 73.15 ± 0.52 | 20.68 ± 2.39 |
| PO1d | 0.44 | 0.44 | 2.26 | 73.77 | 23.08 |
| gsy1Δ | 1.57 ± 0.48 | 0.57 ± 0.62 | 1.24 ± 0.52 | 75.6 ± 1.46 | 21.02 ± 1.74 |
| gsy1Δ-AGPAT1 | 28.7 ± 0.46 | 1.36 ± 0.23 | 3.24 ± 0.03 | 48.98 ± 0.41 | 17.72 ± 0.84 |
| gsy1Δ-CreLPAT | 35.9 ± 1.4 | 3.63 ± 3.79 | 3.33 ± 0.84 | 43.72 ± 1.68 | 13.43 ± 1.2 |
| gsy1Δ-PLPAT | 3.13 ± 0.55 | 1.57 ± 0.43 | 1.06 ± 0.09 | 77.2 ± 1.28 | 17.05 ± 0.32 |

TABLE 14
Total C16:0 enrichment at the sn-2 position in TAG of Y. lipolytica
strains in nitrogen-limited media. Values are means ± SD of
measurements made on three separate cultures for each geneotype.

| Strain | % of C16:0 at sn-2 position |
|---|---|
| WT | 3.53 ± 2.22 |
| PO1d | 0.64 |
| gsy1Δ | 5.5 ± 2.09 |
| gsy1Δ-AGPAT1 | 57.83 ± 2.76 |
| gsy1Δ-CreLPAT | 62.68 ± 6.17 |
| gsy1Δ-PLPAT | 11.6 ± 1.79 |

Optimized Gene sequence synthesis for expression in Y. lipolytica. With reference to the sequences below, the underlined parts show where the coding sequences have restriction sites added at each end and a 3′UTR/terminator.

CreLPAT -
(SEQ ID NO: 36)
AAGCTTATG
TCTGTCCTCACCAAGTGGCTGGGTCTCCCCTCTTTCCTGTTCTCCGTCTT
CGTGTTCTACTGGTCTCTCCCCATCTTCGCCATTCTGTACCGAATCCGAT
TCGCTTCCCTGGGAAAGCGAAACGACATGCTCGACTGGGCTCGAGCCCTG
GTCGCCTACTTCCGAGTGACCCTGCTCCAGGCTGGCGAGCACACCCTGTA
CAAGGGCGGTCCCTGCCTGTACCTCTGTAACCACCGATCCTGGGCTGACT
TCTTCATTGACGCTTACCTGACCGAGGGACGAGCTGCTCTCATGTCTCGA
TGGCTGGTCTACTTCGTGTTCCCCGTCTTCTGCACCTCCTGTATGATCCT
CAAGGGTATTGTCCTGTTCAAGCGAGGAACCATTGCCGACAAGGAAGCCT
TCAACGCCTGGCTGGACCAGACCCTGGGATCCTCTCACGTCCCTGGACTG
CTGGTGTACCCCGAGGGACACCGATCTACCAAGCCTGCCTCCCTGCCTCT
CAAGCGAGGTATGCTCCACTACGCTCACTCTCGAAAGCTGCCCGTGCAGA
TTGTCGTGACCCGAGGCAAGGACGAGGTCCTGTCCGAGAAGTCTCAGTCC
GTGCACTTCGGACGAACCTGCGTCACCACCTTCTCTAAGGTGCTCAAGTC
CGCTGACTACCCCAACTTCGAGGCCTTCTTCACCGACCTGCAGGCTACCT
GGGACTCTTGTTGGGCCGCTACCTACGGACTGGAGGACCTCAAGAACGTG
CCTCGATTCTCTATGCCCGGACCTCAGGCCTACTCCTACTCCTCTTCCAT
GTGGGTGCAGCAGCTCGCCATCACCCTCGTGTCTATTCTGGTCTTCGCTG
GAGTTTGTTACGGCTCCTGGCGAGGTCTGGCCGCTGCCCTGGCTGCTACC
GGTGCTGCCCAGCAGGTGGTTGCTCTGGTGCTGGCTGCTTGGGTGGGTTC
TTCCGTGCTCCGATCCTTCCTGTAACGATCGTTTTTTTTTATATATATAT
ATATATATATATAACTGTCTAGAAATAAAGAGTATCATCTTTCAAAAAGC
TT
AGPAT1 -
(SEQ ID NO: 37)
AAGCTTATG
GACCTGTGGCCCGGAGCTTGGATGCTGCTCCTGCTCCTGTTCCTCCTCCT
GCTGTTCCTCCTGCCCACCCTGTGGTTCTGCTCCCCCTCTGCTAAGTACT
TCTTCAAGATGGCCTTCTACAACGGTTGGATTCTGTTCCTGGCCGTCCTG
GCTATTCCCGTCTGTGCTGTGCGAGGACGAAACGTGGAGAACATGAAGAT
CCTCCGACTGATGCTCCTGCACATCAAGTACCTGTACGGAATTCGAGTTG
AGGTCCGAGGCGCCCACCACTTCCCTCCCTCCCAGCCTTACGTCGTGGTC
TCTAACCACCAGTCCTCTCTGGACCTCCTGGGTATGATGGAGGTGCTCCC
TGGACGATGTGTCCCTATCGCTAAGCGAGAGCTGCTCTGGGCTGGTTCCG
CTGGACTGGCTTGTTGGCTGGCTGGCGTCATCTTCATTGACCGAAAGCGA
ACCGGTGACGCTATTTCCGTGATGTCTGAGGTGGCTCAGACCCTCCTGAC
CCAGGACGTTCGAGTCTGGGTGTTCCCTGAGGGAACCCGAAACCACAACG
GTTCCATGCTGCCCTTCAAGCGAGGCGCCTTCCACCTCGCTGTCCAGGCT
CAGGTCCCTATTGTGCCCATTGTCATGTCCTCTTACCAGGACTTCTACTG
CAAGAAGGAGCGACGATTCACCTCTGGACAGTGTCAGGTCCGAGTGCTGC
CTCCCGTGCCTACCGAGGGACTGACCCCCGACGACGTTCCTGCTCTGGCT
GACCGAGTCCGACACTCCATGCTGACCGTGTTCCGAGAGATTTCTACCGA
CGGTCGAGGCGGTGGAGACTACCTCAAGAAGCCCGGCGGTGGAGGCTAAC
GATCGTTTTTTTTTATATATATATATATATATATATAACTGTCTAGAAAT
AAAGAGTATCATCTTTCAAAAAGCTT
PLPAT -
(SEQ ID NO: 38)
AAGCTTATG
TCCGACCTGTCTGGTGCTGCTACCCCCGAGTCCACCTACCCTGAGCCTGA
GATCAAGCTGTCCTCTCGACTCCGAGGAATTTGCTTCTGTCTCGTCGCCG
GCGTGTCTGCTATCGTCCTGATTGTGCTCATGATCACCGGCCACCCCTTC
GTCCTGCTCTTCGACCGATACCGACGAAAGTTCCACCACTTCATCGCCAA
GCTGTGGGCTTCCATCTCTATCTACCCCTTCTACAAGACCGACATTCAGG
GTCTGGAGAACCTCCCCTCCTCTGACACCCCCTGCGTCTACGTGTCCAAC
CACCAGTCTTTCCTGGACATCTACACCCTGCTCTCCCTCGGACAGTCTTA
CAAGTTCATTTCCAAGACCGGCATCTTCGTCATTCCCGTGATCGGCTGGG
CCATGTCCATGATGGGTGTCGTGCCCCTGAAGCGAATGGACCCCCGATCT
CAGGTCGACTGCCTGAAGCGATGTATGGAGCTCGTCAAGAAGGGTGCCTC
CGTCTTCTTCTTCCCCGAGGGAACCCGATCTAAGGACGGACGACTGGGCC
CCTTCAAGAAGGGCGCTTTCACCATTGCTGCTAAGACCGGTGTGCCTGTG
GTGCCCATTACCCTGATGGGCACCGGCAAGATCATGCCCACCGGTTCCGA
GGGAATTCTCAACCACGGTGACGTCCGAGTGATCATTCACAAGCCCATCT
ACGGATCTAAGGCTGACCTGCTCTGTGACGAGGCCCGAAACAAGATTGCT
GAGTCCATGAACCTGCTCTCTTAACGATCGTTTTTTTTTATATATATATA
TATATATATATAACTGTCTAGAAATAAAGAGTATCATCTTTCAAAAAGCT
T

### Example 7

Cultivation of Yarrowia lipolytica on Various Carbon Sources

In Example 6, TAG fatty acyl composition and % of C16:0 at the sn-2 position were evaluated in Y. lipolytica strains grown on nitrogen-limited media with 20 g L⁻¹ glycerol as the carbon source. In this example, we additionally evaluated use of sugar, vegetable oil and mixtures of these carbon sources to examine whether there is an effect on % C16:0 at the sn-2 position in TAG, total fatty acyl composition of TAG, total lipid content and biomass formation. The sugar, oil and mixed carbon sources used in this example are as follows:
20 g L⁻¹ glycerol (see Example 6)
20 g L⁻¹ glucose
20 g L⁻¹ palm oil
10 g L⁻¹ glucose and 10 g L⁻¹ palm oil
10 g L⁻¹ glycerol and 10 g L⁻¹ palm oil

The culture media and growth conditions for Y. lipolytica strains were the same as described for Example 6, except that when cells were grown with palm oil, the media was supplemented to 0.1% Tween-80 per gram of oil used. Lipid extraction and analyses were performed as described in Example 1.

### Cultivation on Glucose

The data presented in Table 15 and Table 19 show that when Y. lipolytica WT, gsy 1Δ and gsy 1Δ-CreLPAT strains are supplied glucose as a carbon source in nitrogen-limited media, the TAG fatty acyl profile and % of C16:0 at the sn-2 position (Table 19) are similar to those observed when the strains are cultured on glycerol (Example 6).

TABLE 15
Total and sn-2 fatty acyl composition of TAG of Y. lipolytica strains
in nitrogen-limited media with glucose as the carbon source. Values
are means ± SD of measurements made on three separate cultures
for each genotype. nd is not detected.

| Strain | 16:0 | 18:0 | 16:1 | 18:1 | 18:2 |
|---|---|---|---|---|---|
| | Total fatty acid composition in TAG (%) | | | | |
| WT | 22.5 ± 0.13 | 8.6 ± 0.05 | 6.5 ± 0.15 | 50.1 ± 0.15 | 12.2 ± 0.30 |
| gsy1Δ | 10.2 ± 0.29 | 8.8 ± 0.27 | 4.6 ± 0.18 | 64.6 ± 0.31 | 11.7 ± 0.16 |
| gsy1Δ- | 19.8 ± 0.28 | 8.3 ± 0.12 | 3.5 ± 0.12 | 58.3 ± 0.07 | 10.1 ± 0.20 |
| CreLPAT | | | | | |
| | Fatty acid species at sn-2 position (%) | | | | |
| WT | 0.7 ± 0.37 | 0.6 ± 0.36 | nd | 76.9 ± 0.92 | 20.5 ± 0.22 |
| gsy1Δ | 0.7 ± 0.23 | 0.4 ± 0.09 | nd | 81.3 ± 0.23 | 16.9 ± 0.15 |
| gsy1Δ- | 36.5 ± 0.34 | 0.4 ± 0.04 | 0.8 ± 0.04 | 49.0 ± 0.51 | 10.4 ± 0.16 |
| CreLPAT | | | | | |

### Cultivation on Palm Oil

The data presented in Table 16 and Table 19 show that when Y. lipolytica WT and gsy 1Δ-CreLPAT strains are supplied palm oil as a carbon source in nitrogen-limited media, the TAG fatty acyl profiles are altered relative to culture on glycerol or glucose. The total % of C16:0 in TAG is increased from ˜20% to ˜26% in gsy 1Δ-CreLPAT and the total % of stearic acid (C18:0) is decreased from ˜8% to ˜1%. For gsy 1Δ-CreLPAT, the % of C16:0 at the sn-2 position is similar when cultured on palm oil, glucose or glycerol. However, the % of total long chain saturated fatty acyl groups (C16:0+C18:0) at the sn-1/3 positions is decreased when gsyl4-CreLPAT is cultured on palm oil verses glucose or glycerol, because the total % of C18:0 in TAG is lowered and C18:0 is predominantly esterified at the sn-1/3 positions. In WT and gsy 1Δ-CreLPAT, culture on palm oil also leads to a higher cell biomass, cell lipid content and lipid titre than culture on glucose or glycerol (Table 20).

TABLE 16
Total and sn-2 fatty acyl composition of TAG of Y. lipolytica strains
in nitrogen-limited media with palm oil as the carbon source.
Values are means ± SD of measurements made on
three separate cultures for each genotype. nd is not detected.

| Strain | 16:0 | 18:0 | 16:1 | 18:1 | 18:2 |
|---|---|---|---|---|---|
| | Total fatty acid composition in TAG (%) | | | | |
| WT | 30.3 ± 0.53 | 2.2 ± 0.23 | 5.4 ± 1.14 | 39.2 ± 0.27 | 22.8 ± 0.44 |
| gsy1Δ- | 26.2 ± 0.96 | 1.1 ± 0.09 | 3.7 ± 0.10 | 42.2 ± 0.56 | 26.3 ± 0.67 |
| CreLPAT | | | | | |
| | Fatty acid species at sn-2 position (%) | | | | |
| WT | 0.40 ± 0.07 | 0.3 ± 0.04 | nd | 64.0 ± 0.31 | 35.3 ± 0.21 |
| gsy1Δ- | 48.8 ± 0.93 | 1.0 ± 0.77 | 0.7 ± 0.06 | 37.5 ± 1.04 | 11.7 ± 0.64 |
| CreLPAT | | | | | |

### Cultivation on Glucose and Palm Oil

The data presented in Table 17 and Table 19 show that when Y. lipolytica WT and gsy1Δ-CreLPAT strains are supplied with a mixture of glucose and palm oil as carbon sources in nitrogen-limited media, the TAG fatty acyl profiles are altered relative to culture on glucose or palm oil alone. The total % of C16:0 in TAG is increased in gsy1Δ-CreLPAT (to ˜24%) relative to culture on glucose or glycerol and the total % of C18:0 is also decreased. For gsy 1Δ-CreLPAT, the % of C16:0 at the sn-2 position is higher on glucose+palm oil (˜69%) than it is on glucose, palm oil or glycerol alone (˜62%). The % of total long chain saturated fatty acids (C16:0+C18:0) at the sn-1/3 positions is lower on glucose+palm oil than it is on glucose, palm oil or glycerol alone. Human milk fat (HMF) usually has a total % C16:0 content of 20 to 25% with ˜70% of C16:0 at the sn-2 position and a relatively low total C18:0 content of ˜5%. The TAG fatty acyl composition of gsy1Δ-CreLPAT when cultured on glucose+palm oil is therefore a better substitute, providing an adequate total % of C16:0 while minimising the % of, not only C16:0 but also, total long chain saturated fatty acyl groups (C16:0+C18:0) present at sn-1/3. In WT and gsy1Δ-CreLPAT, culture on glucose+palm oil also leads to a higher cell biomass, cell lipid content and lipid titre than culture on glucose or glycerol (Table 20).

TABLE 17
Total and sn-2 fatty acyl composition of TAG of Y. lipolytica strains in
nitrogen-limited media with a mixture of palm oil and glucose as
the carbon sources. Values are means ± SD of measurements made
on three separate cultures for each genotype. nd is not detected.

| Strain | 16:0 | 18:0 | 16:1 | 18:1 | 18:2 |
|---|---|---|---|---|---|
| | Total fatty acid composition in TAG (%) | | | | |
| WT | 24.5 ± 2.50 | 2.3 ± 0.14 | 3.0 ± 0.35 | 48.6 ± 1.91 | 21.7 ± 0.49 |
| gsy1Δ- | 23.9 ± 3.15 | 1.2 ± 0.09 | 3.2 ± 0.12 | 49.4 ± 2.69 | 21.7 ± 1.17 |
| CreLPAT | | | | | |
| | Fatty acid species at sn-2 position (%) | | | | |
| WT | 0.60 ± 0.48 | 0.5 ± 0.35 | nd | 69.1 ± 1.02 | 29.8 ± 1.19 |
| gsy1Δ- | 49.4 ± 3.76 | 0.5 ± 0.15 | 0.7 ± 0.06 | 39.0 ± 3.94 | 9.80 ± 0.07 |
| CreLPAT | | | | | |

### Cultivation on Glycerol and Palm Oil

The data presented in Table 18, Table 19 and Table 20 show that when Y. lipolytica gsy1Δ-CreLPAT is supplied with a mixture of glycerol and palm oil as carbon sources in nitrogen-limited media, the TAG fatty acyl profile, % of C16:0 at the sn-2 position (Table 19), cell biomass, lipid content and lipid titre are more similar to those observed when the strain is cultured on glucose+palm oil than on glucose or glycerol alone. No dipalmitoyl PC was detected in Y. lipolytica WT or gsy 1Δ-CreLPAT cultured on carbon sources used in Example 6 & 7. Samples were analysed using the method described in Example 1. This suggests that Y. lipolytica cells expressing a C16:0 LPAT exclude C16:0 from the sn-2 position of PC; something that we have also observed in Arabidopsis seeds expressing C16:0 LPATs. Therefore C16:0 incorporation into the sn-2 position of TAG in Y. lipolytica expressing a C16:0 LPAT may also be restricted by enzyme activities responsible for DG/PC conversion, for example C16:0 incorporation into the sn-2 position of TAG could be enhanced by suppressing or preventing the activity of choline phosphotransferase (CPT1; EC 2.7.8.2) and/or ethanolamine phosphotransferase (EPT1; EC 2.7.8.1).

TABLE 18
Total and sn-2 fatty acyl composition of TAG of Y. lipolytica strains
in nitrogen-limited media with a mixture of palm
oil and glycerol as carbon sources. Values are of measurements
made on a single culture for each genotype.

| Strain | 16:0 | 18:0 | 16:1 | 18:1 | 18:2 |
|---|---|---|---|---|---|
| | Total fatty acid composition in TAG (%) | | | | |
| gsy1Δ-CreLPAT | 23.0 | 2.7 | 4.3 | 44.2 | 25.7 |
| | Fatty acid species at sn-2 position (%) | | | | |
| gsy1Δ-CreLPAT | 48.3 | 0.4 | 1.6 | 37.1 | 12.2 |

TABLE 19
Total C16:0 enrichment at the sn-2 position in TAG of Y.
lipolytica strains in nitrogen-limited media with various carbon
sources. Values are mean ± SD of measurements made on three
separate cultures for each genotype, except for gsy1Δ-CreLPAT
on Glycerol + Palm oil where thevalue is for a single culture.

| Strain | Substrate | % of C16:0 at sn-2 position |
|---|---|---|
| WT | Glucose | 1.10 ± 0.56 |
| gsy1Δ | | 2.10 ± 0.77 |
| gsy1Δ-CreLPAT | | 61.6 ± 0.94 |
| WT | Palm oil | 0.50 ± 0.07 |
| gsy1Δ-CreLPAT | | 62.2 ± 3.15 |
| WT | Glucose + Palm oil | 0.90 ± 0.66 |
| gsy1Δ-CreLPAT | | 69.1 ± 3.98 |
| gsy1Δ-CreLPAT | Glycerol + Palm oil | 69.9 |

TABLE 20
Y. lipolytica strain biomass, lipid content and lipid titre in
nitrogen-limited media with various carbon sources.
Values are means ± SD of measurements made on three
separate cultures for each genotype, except for gsy1Δ-CreLPAT
on Glycerol + Palm oil where the value is for a single culture.

| | | Biomass | Lipid (mg g⁻¹ | Lipid |
|---|---|---|---|---|
| Strain | Substrate | (g L⁻¹) | Biomass) | (g L⁻¹) |
| WT | Glucose | 4.0 ± 0.13 | 101.3 ± 17.84 | 0.4 ± 0.06 |
| gsy1Δ | | 3.6 ± 0.06 | 181.6 ± 3.44 | 0.7 ± 0.01 |
| gsy1Δ- | | 3.7 ± 0.05 | 170.5 ± 16.73 | 0.6 ± 0.06 |
| CreLPAT | | | | |
| WT | Glycerol | 4.0 ± 0.13 | 115.2 ± 5.39 | 0.5 ± 0.04 |
| gsy1Δ- | | 3.3 ± 0.07 | 215.1 ± 9.11 | 0.7 ± 0.04 |
| CreLPAT | | | | |
| WT | Palm oil | 6.3 ± 1.06 | 480.3 ± 65.06 | 3.1 ± 0.81 |
| gsy1Δ- | | 6.7 ± 1.29 | 465.7 ± 40.53 | 3.1 ± 0.48 |
| CreLPAT | | | | |
| WT | Glucose + Palm oil | 5.9 ± 0.23 | 424.5 ± 21.98 | 2.5 ± 0.09 |
| gsy1Δ- | | 5.9 ± 1.27 | 468.1 ± 21.69 | 2.8 ± 0.50 |
| CreLPAT | | | | |
| gsy1Δ- | Glycerol + Palm oil | 4.84 | 405 | 1.96 |
| CreLPAT | | | | |

### Example 8

Although the examples discussed above show that plant lipid metabolism can be engineered to preferentially esterify 16:0 to the sn-2 position in TAG, it would be desirable for the total fatty acid composition of the Arabidopsis seeds to more closely resemble that of human milk. 16:0 is ˜3-fold less abundant in Arabidopsis seeds and they contain a high proportion of polyunsaturated and very-long-chain fatty acid species that are essentially absent from human milk. The most abundant fatty acid in human milk is 18:1 and, because of the unusual regiospecific distribution of the next most abundant fatty acid 16:0, the major molecular species of TAG is usually 1,3-olein-2-palmitin (OPO) accounting for ˜14% of the total. We decided to investigate whether Arabidopsis seeds can be engineered to produce OPO, by combining 16:0 enrichment at the sn-2 position in TAG with a total fatty acid composition rich in the appropriate ratio of 16:0 and 18:1.

### Results

Seed of fab1-1 fae1 fad2 Are High in 16:0 and 18:1 (HPHO)

To obtain Arabidopsis seeds with 16:0 content equivalent to human milk the level must be increase ˜3-fold to 20-25%. One approach to achieve this is to reduce fatty acid synthase catalysed 16:0 elongation by disrupting the β-ketoacyl-ACP synthase II gene FATTY ACID BIOSYNTHESIS 1 (FAB1) (FIG. 8). FAB1 is an essential gene in Arabidopsis, but a single hypomorphic fab1-1 allele has been characterised that contains ˜17% 16:0 in is seeds. 16:0 can then be increased to ˜24% in a fatty acid elongase 1 (fae1) mutant background, which is deficient in very-long-chain fatty acid synthesis (FIG. 8). However, fab1-1 fae1 seeds still contain a high proportion of polyunsaturated fatty acids, produced via FATTY ACID DESATURASE 2 (FAD2) (FIG. 8). To create a background high both 16:0 and 18:1 (HPHO) we therefore constructed a fab1-1 fae1 fad2 mutant by crossing. Analysis of homozygous fab 1-1 fae1 fad2 seeds showed that the fatty acid composition of the TAG is high in 16:0 and 18:1, which account for ˜20 and ˜70% of total fatty acid, respectively (FIG. 9). Other fatty acid species that are normally abundant in wild type Arabidopsis Col-0 seed TAG, such as linoleic acid (18:2), linolenic acid (18:3) and eicosenoic acid (20:1), each account for <3% (FIG. 9). Comparison with the double mutants showed that 16:0 content in fab1-1 fae1 is reduced significantly (P>0.05) in the fad2 background (FIG. 9). Nevertheless, the HPHO composition of fab1-1 fae 1 fad2 seeds suggests that this genetic background is appropriate to test whether OPO can be produced in seeds.

ΔCTS-LPAT1 Expression in HPHO Seed Drives 16:0 Incorporation Into the sn-2 Position of TAG

As discussed above, we have shown that expression of an ER-retargeted version of the chloroplast LPAT (ΔCTS-LPAT1) in WT Arabidopsis seeds, under the soybean glycinin-1 promoter (ProGLY), leads to a substantial increase in esterification of 16:0 to the sn-2 position in TAG. To determine what effect ΔCTS-LPAT1 expression has in a HPHO background, we constructed a ProGLY:ΔCTS-LPAT1 fab1-1 fae1 fad2 line by crossing. When we purified TAG from the seeds and determined its regiochemistry, we found that the percentage of 16:0 at the sn-2 position (versus sn-1+3), had increased from ˜3% in the fab1-1 fad2 fae1 background to ˜24% in ProGLY:ΔCTS-LPAT1 fab1-1 fad2 fae1 (FIG. 10A). The total fatty acid composition of fab1-1 fad2 fae1 seeds was not altered greatly by ΔCTS-LPAT1 expression, except that there was a small but significant (P>0.05) increase in 16:0 abundance, from ˜19 to ˜22% (FIG. 10B). We previously observed a small increase in total 16:0 content when we expressed ΔCTS-LPAT1 in WT seeds. Our data show that ΔCTS-LPAT1 expression in HPHO seeds allows incorporation of 16:0 into the sn-2 position of TAG. However, the level of enrichment is significantly lower (P>0.05) than in WT seeds containing ProGLY:ΔCTS-LPAT1 (FIG. 10A).

AGPAT1 Expression Drives Stronger 16:0 Incorporation Into the sn-2 Position of TAG

To investigate whether other ER-localised LPATs might enable Arabidopsis to incorporate more 16:0 into the sn-2 position of TAG than ΔCTS-LPAT1, we decided to test Homo sapiens AGPAT1. Human milk fat globules are secreted by lactocytes in the mammary gland epithelium. It is not known which LPAT is responsible for human milk fat biosynthesis. However, AGPAT1 is expressed in mammary epithelial cells and in vitro assays suggest that AGPAT1 can use 16:0-CoA as a substrate. To test whether AGPAT1 can incorporate 16:0 into the sn-2 position of TAG we first expressed the protein in Saccharomyces cerevisiae under the GAL1 promoter (28). When we purified TAG and determined its regiochemistry, we found that the percentage of 16:0 at the sn-2 position had increased from ˜4% in cells harbouring an empty vector control to ˜45% in cells containing ProGAL1:AGPAT1 (Table 21).

TABLE 21
16:0 in TAG from S. cerevisiae cells expressing AGPAT1.

| Vector | 16:0 at sn-2 (%) | Total 16:0 (%) |
|---|---|---|
| pYES2 (EVC) | 3.8 ± 0.7 | 11.8 ± 0.2 |
| pYES2 + AGPAT1 | 44.7 ± 3.9* | 14.6 ± 0.3* |

Values are the mean ± SE of measurements on lipid extracts from three separate cultures.
*denote values significantly (P < 0.05) different either from empty vector control (EVC) (two-tailed Student′s t-tests).

Using transient expression in Nicotiana benthamiana leaves, we also confirmed that AGPAT1 can localise to the ER in plant cells when it is expressed as a red fluorescent protein (RFP)-AGPAT1 fusion protein under the cauliflower mosaic virus 35S promoter (FIG. 11A). Next we transformed WT Arabidopsis plants with a ProGLY:AGPAT1 construct in order drive strong seed-specific expression of the transgene. From >40 primary transformants we selected two independent single copy T2 lines (L35 & L40) for analysis and obtained homozygous T3 seed. When we purified TAG from the homozygous seed batches and determined its regiochemistry, we found that the percentage of 16:0 at the sn-2 position was ˜66% for L40 and ˜74% for L35 (FIG. 11B). To determine what effect AGPAT1 expression has in the HPHO background we constructed a ProGLY:AGPAT1 fab1-1 fae1 fad2 line by crossing. When we purified TAG from these seeds and determined its regiochemistry, we found that the percentage of 16:0 at the sn-2 position was ˜54% (FIG. 11B). The total 16:0 content of ProGLY:AGPAT1 fab1-1 fae1 fad2 seeds was not significantly different (P>0.05) from fab1-1 fae1 fad2 (FIG. 11C). AGPAT1 expression therefore allows a higher incorporation of 16:0 into the sn-2 position of TAG in WT and HPHO seeds than ΔCTS-LPAT1.

Disruption of LPAT2 and PDCT Enhances ΔCTS-LPAT1 and AGPAT1-Dependent 16:0 Incorporation Into the sn-2 Position of TAG in HPHO Seeds

In wild type (WT) Arabidopsis seeds we previously found that ΔCTS-LPAT1-dependent incorporation of 16:0 into the sn-2 position of TAG could be increased by disrupting the enzymes LPAT2 and PDCT. LPAT2 is the main ER-localized LPAT isoform expressed in Arabidopsis seeds and therefore disruption likely reduces competition with ΔCTS-LPAT1 (FIG. 8). PDCT catalyses rapid DG/PC interconversion in Arabidopsis seeds. Although LPAT initially acylates glycerolipids at sn-2, once these acyl groups are in PC they can be removed and replaced by a deacylation-reacylation (acyl editing) cycle. Disruption of PDCT forces a more direct flux of newly made DG into TAG (FIG. 8). To determine whether LPAT2 and PDCT disruption affect the percentage of 16:0 esterified to the sn-2 position in TAG in HPHO seeds expressing ΔCTS-LPAT1 or AGPAT1, we constructed ProGLY: ΔCTS-LPAT1 fab1-1 fae1 fad2 lpat2-2 pdct and ProGLY:AGPAT1 fab1-1 fae1 fad2 lpat2-2 pdct lines by crossing. When we purified TAG from these seeds and determined its regiochemistry, we found that the percentage of 16:0 at the sn-2 position, was ˜62% and ˜83%, respectively (FIG. 12A). The total 16:0 content in ProGLY:ΔCTS-LPAT1 fab 1-1 fae1 fad2 lpat2-2 pdct and ProGLY:AGPAT1 fab 1-1 fae1 fad2 lpat2-2 pdct seeds was ˜21 and ˜18%, respectively (FIG. 12B).

HPHO Seeds Have Reduced Oil Content and Seed Vigour but This Is not Compounded by Redistribution of 16:0 to the sn-2 Position

Modification of fatty acid composition can reduce TAG accumulation in oilseeds and can also impair seed germination and seedling establishment. As discussed above, we have found that ProGLY: ΔCTS-LPAT1 lpat2-3 pdct seeds, which have a low total 16:0 content but ˜70% esterified to the sn-2 position, exhibit a reduction in TAG content as a percentage of seed weight. However, their germination and initial seedling growth were not significantly impaired. To examine the physiological impact of 16:0 enrichment at the sn-2 position of TAG in HPHO seeds, we compared seed batches from WT, fab1-1 fae1 fad2, ProGLY:ΔCTS-LPAT1 fab1-1 fae1 fad2 lpat2-2 pdct and ProGLY:AGPAT1 fab1-1 fae1 fad2 lpat2-2 pdct plants that had been grown together under standard laboratory conditions. We found that both seed weight and percentage oil content were significantly reduced (P>0.05) in fab1-1 fae1 fad2 relative to WT (FIG. 13). However, no significant difference was observed between fab1-1 fae1 fad2 and ProGLY:ΔCTS-LPAT1 fab1-1 fae1 fad2 lpat2-2 pdct or ProGLY:AGPAT1 fab1-1 fae1 fad2 lpat2-2 pdct seeds (FIG. 13). These data suggest that TAG biosynthetic flux is impaired in fab1-1 fae1 fad2 seeds, but that the further genetic modifications leading to incorporation of 16:0 into the sn-2 position of TAG are not detrimental in this background. These findings contrast with what we observed in WT seeds. In standard laboratory conditions (20° C., 16 h photoperiod), the speed of fab1-1 fae1 fad2 seed germination (scored as radicle emergence) and seedling establishment (scored as cotyledon expansion) was significantly slower (P>0.05) than WT (FIG. 14). However, no significant difference (P>0.05) was observed between fab1-1 fae1 fad2 and ProGLY:ΔCTS-LPAT1 fab1-1 fae1 fad2 lpat2-2 pdct or ProGLY:AGPAT1 fab1-1 fae1 fad2 lpat2-2 pdct seeds (FIG. 14).

### Discussion

In this example we show that Arabidopsis seeds can be engineered to produce OPO, since the TAG contains ˜20% 16:0 and ˜70% 18:1, with >80% of the 16:0 esterified to the sn-2 position on the glycerol backbone, by combining fab1-1, fae1 and fad2 alleles. OPO is the main TAG species present in human milk, but it is virtually absent from vegetable oils, which typically contain very little 16:0 esterified to the sn-2 position. The high OPO content of human milk is believed to confer nutritional benefits and therefore the development of a vegetable oil that is rich in OPO could provide a useful new source of ingredient for infant formulas.

The expression of an ER-retargeted version of the chloroplast LPAT (ΔCTS-LPAT1) in WT Arabidopsis seeds allows ˜30 to 40% of the 16:0 present in the TAG to occupy the sn-2 position. However, when we expressed ΔCTS-LPAT1 in the HPHO background, we found the incorporation of 16:0 into the sn-2 position was reduced to ˜20%. Disruption of LPAT2 and PDCT then lead to an increase in the percentage of 16:0 at sn-2 to ˜62%. This level of 16:0 enrichment at sn-2 is also lower than we were able to achieve in WT using the same approach. However, the total fatty acid composition of HPHO is far more appropriate for an infant formula ingredient and ˜62% 16:0 at sn-2 still compares favourably with commercially available HMFS that are produced by in vitro enzyme-catalysis.

Generally, in human milk fat >70% of the 16:0 is esterified to the sn-2 position of TAG and this level of enrichment therefore remains the target for HMFS. The human LPAT AGPAT1 can use 16:0-CoA as a substrate and is expressed in lactocytes. When we expressed AGPAT1 in WT Arabidopsis seeds we found that 60 to 70% of the 16:0 present in the TAG occupied the sn-2 position.

When we expressed AGPAT1 in a HPHO background, incorporation of 16:0 into the sn-2 position was reduced to ˜54%, but disruption of LPAT2 and PDCT then lead to an increase in the percentage of 16:0 at sn-2 to ˜83%. This level of enrichment of 16:0 at sn-2 is greater than or equal to that reported in human milk fat.

Metabolic pathway engineering can often have detrimental effects on TAG accumulation in oilseeds and can impair seed vigour. In EXAMPLE 1 discussed above, we found that redirecting 16:0 to the sn-2 position of TAG in WT Arabidopsis seeds reduced oil accumulation. HPHO seeds also have a lower seed oil content than WT. However, engineering a similar shift in positional distribution of 16:0 did not lead to a further reduction. Given that WT seeds have a ˜3-fold lower 16:0 content than HPHO seeds, it may be that 16:0 availability restricts the rate of TAG biosynthesis in seeds engineered to only possess 16:0-CoA LPAT activity. Conversely, the ˜3-fold higher 16:0 content in HPHO seeds might conceivably restrict TAG biosynthesis because native LPATs (and other acyltransferase activities) have too little 16:0-CoA activity. HPHO seeds are also significantly impaired in seed germination and early seedling growth. However, redirecting 16:0 to the sn-2 position of TAG in HPHO seeds does not compound this effect. Poorer HPHO seed vigour may be caused by the reduction in long-chain fatty acid unsaturation, which raises the melting temperature of the oil. This property is not greatly influenced by the positional distribution of the fatty acid.

### Materials and Methods

### Plant Material and Growth Conditions

The Arabidopsis thaliana Colombia-0 mutants fab1-1, fae1, fad2, pdct and lpat2-3 have been described previously (26) (27) (2). For experiments performed on media, ˜50 seeds from individual plants were surface sterilized, plated on agar plates containing one-half strength Murashige and Skoog salts (Sigma-Aldrich) pH 5.7 and imbibed in the dark for 4 d at 4° C. The plates were then placed in a growth chamber set to 16-h light (photosynthetic photon flux density=150 μmol m⁻² s⁻¹)/8-h dark cycle at a constant temperature of 20° C. Germination (radicle emergence) and cotyledon expansion was scored visually under a dissecting stereomicroscope as described previously herein. Individual seedlings were also transplanted to 7 cm² pots containing Levington F2 compost and grown in a chamber set to a 16-h light (22° C.)/8-h dark (16° C.) cycle, with a photosynthetic photon flux density of 250 μmol m⁻² s⁻¹. The plants were bagged individually at the onset of flowering and the seeds were harvested at maturity.

### Genotyping

Genomic DNA was isolated from leaves using the DNeasy Plant Mini Kit (Qiagen). Homozygous lpat2-3 T-DNA insertional mutants were identified by PCR using Promega PCR Master Mix (Promega) and combinations of the gene specific and T-DNA left border primers pairs, as described previously herein. Homozygous fab1-1, fad2, fae1 and pdct mutants were identified by sequencing PCR products amplified with primer pair spanning the sites of the point mutations (26) (27) (2). The presence of ProGLY:ΔCTS-LPAT and ProGLY:AGPAT1 T-DNAs was determined by PCR using a primer pair spanning ProGLY and ΔCTS-LPAT1 or AGPAT1, as described previously herein.

### Lipid Analysis

Total lipids were extracted from material and TAG was purified as described previously (6). TAG regiochemical analysis was performed by lipase digestion following the method described previously herein, except that 2-monoacylglycerols were separated by thin layer chromatography (Silica gel 60, 20×20 cm; Sigma-Aldrich/Merck) using hexane:diethylether:acetic acid (35:70:1.5, v/v/v) (8). Fatty acyl groups present in whole seeds and purified lipid fractions were trans-methylated and quantified by gas chromatography (GC) coupled to flame ionization detection, as described previously herein, using a 7890A GC system fitted with DB-23 columns (30 m×0.25 mm i.d.×0.25 μm) (Agilent Technologies). Seed oil and moisture contents of whole seeds were measured by low-resolution time domain NMR spectroscopy using a Minispec MQ20 device (Bruker) fitted with a robotic sample-handling system (Rohasys) as described previously herein and the percentage oil content was normalised to 9% moisture.

### Cloning and Transformation

H. sapiens AGPAT1 (GenBank: NP_001358367, SEQ ID NO:40) was codon optimised for expression in Arabidopsis, synthesised by Genscript and supplied in pUC57. AGPAT1 was then amplified by PCR with KOD DNA polymerase (Merck) using primer pair 5′-CACCATGGATTTATGGCCTGGTGC-3′ (SEQ ID NO:74) & 5′-TCATCCTCCTCCACCTGG-3′ (SEQ ID NO:75). The resulting PCR product was purified with the QIAquick Gel Extraction Kit (Qiagen). The PCR product was cloned in the pENTR/D-TOPO vector (Thermo Fisher Scientific), sequenced (SEQ ID NO:26) and recombined into pYES-DEST52 (Invitrogen) and pK7WGR2 (Vlaams Institute for Biotechnology) using the Gateway LR Clonase II Enzyme mix (Thermo Fisher Scientific). AGPAT was cloned in the pBinGly Red3 vector in between the soybean glycinin-1 (GLY) promoter and terminator for seed specific expression (3). AGPAT1 was PCR-amplified from the pENTR-D-TOPO vector using KOD DNA polymerase and primer pair 5′-CGGAATTCATGGATTTATGGCCTGGTGC-3′ (SEQ ID NO:76) & 5′-GCTCTAGATCATCCTCCTCCACCTGG-3′ (SEQ ID NO:77). The PCR product was gel purified and digested with EcoRI and XbaI. The pBinGlyRed3 vector was also digested with EcoRI and XbaI, alkaline phosphatase treated (Promega), gel purified and AGPAT1 was ligated into the vector using T4 DNA ligase (NEB). pYES-DEST52 was transformed into S. cerevisiae INVSc1 cells using the S. c. EasyComp kit (Invitrogen) and protein expression was induced as described by Kim et al., (2005) (28). Heat shock was used to transform the pK7WGR2 and pBinGlyRed3 vectors into Agrobacterium tumefaciens strain GV3101. Arabidopsis transformation was carried out using the floral-dip method (4). T1 seeds expressing the selectable marker were identified under a Leica M205 FA microscope using the DsRed filter.

Transient Expression in N. benthamiana and Imaging

Transient expression in N. benthamiana leaves was carried out as described by Wood et al., (2009) (5) using A. tumefaciens cultures transformed with vectors harbouring Pro35S:RFP-AGPAT1, Pro35S:m-GFP5-ER or Pro35S:p19. Cultures were hand-infiltrated into leaves and the inoculated plants were left for 48 h. N. benthamiana leaves were then mounted in water on a Zeiss LSM 780 laser scanning confocal microscope under an Apochromat 63×/1.20 W Korr M27 objective. GFP was excited at a wavelength of 488 nm and RFP at 561 nm. Filters with an emission band at 473-551 nm were used for detection.

### Statistical Analyses

All experiments were carried out using three biological replicates and the data are presented as the mean values±standard error of the mean (SE). For statistical analysis we either used one-way analysis of variance (ANOVA) with post-hoc Tukey HSD (Honestly Significant Difference) tests, or two-tailed Student's t-tests.

### Accession Numbers

Sequence data described herein can be found in the GenBank/EMBL data libraries under, for example, accession numbers: NP_001358367 (AGPAT1), AF111161 (LPAT1), At1g74960 (FAB1), At3g12120 (FAD2), At4g34520 (FAE1), At3g57650 (LPAT2), At3g15820 (PDCT).

### APPENDIX—ADDITIONAL SEQUENCE INFORMATION

>HsAGPAT1 cDNA sequence
(SEQ ID NO: 39)
ATGGATTTGTGGCCAGGGGCATGGATGCTGCTGCTGCTGCTCTTCCTGCTGCTGCTCTTCCTGCTGCCCA
CCCTGTGGTTCTGCAGCCCCAGTGCCAAGTACTTCTTCAAGATGGCCTTCTACAATGGCTGGATCCTCTT
CCTGGCTGTGCTCGCCATCCCTGTGTGTGCCGTGCGAGGACGCAACGTCGAGAACATGAAGATCTTGCGT
CTAATGCTGCTCCACATCAAATACCTGTACGGGATCCGAGTGGAGGTGCGAGGGGCTCACCACTTCCCTC
CCTCGCAGCCCTATGTTGTTGTCTCCAACCACCAGAGCTCTCTCGATCTGCTTGGGATGATGGAGGTACT
GCCAGGCCGCTGTGTGCCCATTGCCAAGCGCGAGCTACTGTGGGCTGGCTCTGCCGGGCTGGCCTGCTGG
CTGGCAGGAGTCATCTTCATCGACCGGAAGCGCACGGGGGATGCCATCAGTGTCATGTCTGAGGTCGCCC
AGACCCTGCTCACCCAGGACGTGAGGGTCTGGGTGTTTCCTGAGGGAACGAGAAACCACAATGGCTCCAT
GCTGCCCTTCAAACGTGGCGCCTTCCATCTTGCAGTGCAGGCCCAGGTTCCCATTGTCCCCATAGTCATG
TCCTCCTACCAAGACTTCTACTGCAAGAAGGAGCGTCGCTTCACCTCGGGACAATGTCAGGTGCGGGTGC
TGCCCCCAGTGCCCACGGAAGGGCTGACACCAGATGACGTCCCAGCTCTGGCTGACAGAGTCCGGCACTC
CATGCTCACTGTTTTCCGGGAAATCTCCACTGATGGCCGGGGTGGTGGTGACTATCTGAAGAAGCCTGGG
GGCGGTGGGTGA
>HsAGPAT1 amino acid sequence
(SEQ ID NO: 40)
MDLWPGAWMLLLLLFLLLLFLLPTLWFCSPSAKYFFKMAFYNGWILFLAVLAIPVCAVRGRNVENMKILRLMLLHI
KYLYGIRVEVRGAHHFPPSQPYVVVSNHQSSLDLLGMMEVLPGRCVPIAKRELLWAGSAGLACWLAGVIFIDRKRT
GDAISVMSEVAQTLLTQDVRVWVFPEGTRNHNGSMLPFKRGAFHLAVQAQVPIVPIVMSSYQDFYCKKERRFTSG
QCQVRVLPPVPTEGLTPDDVPALADRVRHSMLTVFREISTDGRGGGDYLKKPGGGG
>CreLPAT cDNA sequence (Chlamydomonas reinhardtii gene Cre17.g738350)
(SEQ ID NO: 41)
ATGTCCGTGCTCACAAAGTGGCTGGGCCTGCCCTCATTCTTGTTCTCCGTGTTTGTGTTCTACTGGAGTTTGCCC
ATCTTTGCGATCCTATATCGTATCC
GCTTCGCCTCTCTGGGGAAGCGCAATGATATGCTCGACTGGGCCCGCGCGCTCGTCGCCTACTTCCGAGTAAC
GCTCCTGCAGGCGGGGGAGCACACGCT
GTACAAGGGAGGGCCATGCCTGTACCTGTGCAACCACCGCAGCTGGGCGGACTTCTTCATTGACGCCTACCTC
ACTGAGGGCCGCGCGGCGCTCATGAGC
AGGTGGCTGGTGTATTTCGTGTTCCCAGTGTTCTGTACCTCGTGCATGATCCTCAAGGGGATAGTGCTGTTCAA
GCGCGGCACCATTGCGGATAAAGAGG
CCTTCAACGCCTGGCTTGACCAGACCCTGGGCAGCAGCCACGTGCCTGGGCTGCTGGTGTACCCCGAGGGCCA
CCGCAGCACCAAGCCGGCCAGCCTGCC
GCTGAAGCGCGGCATGCTGCACTACGCGCACAGCCGCAAGCTGCCGGTGCAGATTGTCGTGACTCGCGGCAAG
GACGAGGTGCTGAGTGAGAAGTCGCAG
TCGGTGCACTTCGGCCGCACCTGCGTCACCACCTTCTCCAAGGTGCTCAAGTCGGCCGACTACCCCAACTTTGA
AGCCTTCTTCACGGACCTGCAGGCGA
CCTGGGACTCGTGCTGGGCAGCCACGTACGGCCTGGAGGATCTCAAAAACGTGCCGCGCTTCTCCATGCCGGG
GCCCCAGGCCTACAGCTACAGCTCCAG
CATGTGGGTCCAGCAGCTCGCCATCACGCTGGTCAGCATCCTAGTCTTTGCGGGCGTGTGCTACGGCTCCTGGC
GCGGCTTGGCGGCGGCGCTGGCGGCG
ACCGGCGCGGCGCAGCAGGTGGTGGCACTGGTGCTGGCGGCCTGGGTGGGCAGCTCCGTGCTTCGTAGCTTCC
TCTAG
>CreLPAT amino acid sequence
(SEQ ID NO: 42)
MSVLTKWLGLPSFLFSVFVFYWSLPIFAILYRIRFASLGKRNDMLDWARALVAYFRVTLLQAGEHTLYKGGPCLYL
CNHRSWADFFIDAYLTEGRAALMSRWLVYFVFPVFCTSCMILKGIVLFKRGTIADKEAFNAWLDQTLGSSHVPGLL
VYPEGHRSTKPASLPLKRGMLHYAHSRKLPVQIVVTRGKDEVLSEKSQSVHFGRTCVTTFSKVLKSADYPNFEAFF
TDLQATWDSCWAATYGLEDLKNVPRFSMPGPQAYSYSSSMWVQQLAITLVSILVFAGVCYGSWRGLAAALAATG
AAQQVVALVLAAWVGSSVLRSFL
>LC107611.1 Nannochloropsis sp. NIES-2145 LPAT2 mRNA for lysophosphatidate
acyltransferase 2, complete cds
(SEQ ID NO: 43)
ATGTCTGGTCCAGCCGATGCCAGGGGCCCTGCCCGAGTATCCGATACCATTATTGGTGCGCCCGACCGCC
ACCATGTCGGTCTGACCATCCTCGTCGCCCTTCTCCTGAGCGCCTTTTGCTTTTGCGAATTCTTCATGCT
GCCCGTGGCGATGGCGTTACTGCCCGTGCCAGGTACAACATCGAGGAAGTTGCTGCGGCGCTGGGAGGGC
TTGGTAGCCAAAACTTGGCTGTCCTTTGGAGGGTGGCTCCTGGAAAATGTGGGTGGGGTCAAGCTCATTC
TTACGGGCGACTCGATACTCCCCGGTGATCGTGTGCTGATTCTCAGCAATCATAGGACGAGAATTGATTG
GATGTTTCTATGGTGCTGGGCTGCACGTTTCGACCTGCTTTCCTCGTACCGAGTCATTCTCAAGTCGTCT
TTACGCACTTTCCCCTGGTGGGGCTGGGGGATGAGTTTATGTTTGTTTCCTTTCATCCACCGAGGAAGCA
ACAATCGCGACGCGGATTTGACCCGTATTGAACATATTTGTCAGTATTTAAGCGAGCTGGGTGTGCCAAA
CTCCCTCATTCTCTTCCCAGAAGGAACTGACCTTTCCCAATCTAACCAAAAACGCGATCGTGATTATGCT
CTAGCCAAAAACCTACCCATTTACCATAACGTGCTTCACCCACGTTCAGGCGCCTTTATTGCTAGCTTGA
CGGCCATGCGTCCTCATTTGGATGCTGTTGTGGATCTTACTATCGGCTATGTCGATTACACACCAGGGGA
AAGACCTTCCGAGCTCTCTCTTCTCAAGGGCCGGCTACCGCACGAGGTCCACATAAATATGAAACGATGG
GACATAAAGACGACTCCCTTACTTCGAGAGAACCCATCGGACCGGGCGGAACAATTTTTGCGCGAATCTT
TTGATCGGAAAGAGGCATTGCTGACAGCCTTCTACAGCAACAACACGGACACTTCAGGTGGTGGGCTCAA
GCAAACGTCATCATTGCTCGTATCCTCTTCCTCTTCACCTTTCTCCAATAGCAGTGCACCTTTAGGATTT
CCTTCCTTCTCCTCCCCCTCCACCTCCGTCGCCTCTTCTTACAACGAGCCAGTGTATGTACACAATGCTC
ACTGGACATACGGCAAAGGGCTTTTGGGGACGGGGATTGCCATGGCGTTGGTGCTGATGTTAGCGTGCTG
CTTGCCTGCTGTTGCGTTTTGCGGGTATAGTATCGTTGTCTTTTTGTTGTTCGTGACAGTGAATTCGGTG
TGGGAGGGCTTCGATACCTTGGAATTGAATTTGGCTGTGTGGTGGGGGGGGCAGGCAGGGCAAACGAAGC
AAGAGTGA
>Nannochloropsis sp. NIES-2145 LPAT2 amino acid sequence
(SEQ ID NO: 44)
MSGPADARGPARVSDTIIGAPDRHHVGLTILVALLLSAFCFCEFFMLPVAMALLPVPGTT
SRKLLRRWEGLVAKTWLSFGGWLLENVGGVKLILTGDSILPGDRVLILSNHRTRIDWMFL
WCWAARFDLLSSYRVILKSSLRTFPWWGWGMSLCLFPFIHRGSNNRDADLTRIEHICQYL
SELGVPNSLILFPEGTDLSQSNQKRDRDYALAKNLPIYHNVLHPRSGAFIASLTAMRPHL
DAVVDLTIGYVDYTPGERPSELSLLKGRLPHEVHINMKRWDIKTTPLLRENPSDRAEQFL
RESFDRKEALLTAFYSNNTDTSGGGLKQTSSLLVSSSSSPFSNSSAPLGFPSFSSPSTSV
ASSYNEPVYVHNAHWTYGKGLLGTGIAMALVLMLACCLPAVAFCGYSIVVFLLFVTVNSV
WEGFDTLELNLAVWWGGQAGQTKQE
>LC107612.1 Nannochloropsis sp. NIES-2145 LPAT3 mRNA for
lysophosphatidate acyltransferase 3, complete cds
(SEQ ID NO: 45)
ATGTGGGGCTTATTATGGAAACACATGACCCTACGACTCCTCTTCGTCTCAGTCTGCTTGGTCACCTCCT
CCATCGGCGCCAACATGATGGCCCTCGCCCTCTTCCTCGTCATCCGACCCTTTTCCCGCTCGCTGTATCG
ACGGTTGGTGTCACAGTGTGTGGCATGCATGTGGATCGATGCACTGTCCTTGCTCCTACCGGGAACCAAC
ATCCACATTGCTGCTGACTCTGACATGCCCGACGGGATCACTGCCGGCATCGTGGTGGCCAATCACCAAT
ACGAAGGTGATTGGTGGTTCATGCTCATGGTCGCCCGCTTCCTGGGCCTGCATGGGAACGTGAAGATAAT
AGTCAGGGAGGGCCTTAAGAAGATCCCCCTCTTGGGGTGGCTGGTCCGGCTTGTCGAGTACCCATTCATC
TCCTCCTCCTGGTCGCTCTCCCGCACGAACCTCTTTGGGCTGCTGCGGAGTTTCAATGCGGACGACTTCC
CCGTCCTCCTCTTCCAGTTCCCCGAGGGCGATCGCATCGATGCCAAAGTCCGTCAGCAATCGCTTGCCTT
TGCGGCCAAGGAGCAACGCCCGCACCTCCTCCACGTCCTCCTCCCCCGTACTACCGGTTTCAACACCTGC
ATCGAAGCGCTTAGGACTTCCCATCCCCCCGTCTATGATATGACAATTGCCATCCCTGGCACCACGGGCC
AGCCTTCCTCCTCCTCCTCCTCCTCCTCACCTTCTGCTGCAGCTGCTGCGGCGTCTCCATCTGCTGGTGC
GGCGGCGGCGGCGGCGGCGGCGAGCAACACATCAACCAACCCTTCTTCCAACCCTCCCTCGAGCAGCAGC
ACCGATACTAGTCCAGCTGCTGCTGCTGCTGCTGCTGCTGATGCCGCTGCTGCCGAGAGTCATGACGCTT
CTCTCTTTCACACCTTCCTCCGTTTCTGCAACGGCGAGGGCCCACGGGACGTGCATATCAGACTCAAGCG
CTACTCCCTCAACGACGTCCTGGCCGACCCCCACTGGCTCGACACCAAATGGGCCGAGAAAGACCGCGTC
TTGACCTACTTTTCCCGCCACGCCTGCTTTCCTGCTCCGCTCCCTCCCCACATGGCCAATGGTGGGGGCA
GCACTCAGACACACGGGGGGAGGGGGGGCCCCAGAGGGCCGGCGGGCTCTCATGCCGTGGTGGGAGCAGC
GGGTAACGCCCAGTATCTCCGGTCGTTCAATTCCCGGAAATTCAAAGCAGAAACGAGTTTTTTGGCGTTG
GCACGACTGTTACTCACGCCGTTGTGCCTTCCCTTGTTGTTGCTGATGGCCTGTCCGCTCATGACTCTGT
ATGTATGCGTGAGCACCGTCCGAAGGTGGTTGGGGGAGGAGATTGTGTTCTCGCCAGGAGGACGGGAGGG
AGGGAGGGAGGGCGACGCCGTTTACGAGTCCGTTAATTGGAGCGTTTGA
>Nannochloropsis sp. NIES-2145 LPAT3 amino acid sequence
(SEQ ID NO: 46)
MWGLLWKHMTLRLLFVSVCLVTSSIGANMMALALFLVIRPFSRSLYRRLVSQCVACMWID
ALSLLLPGTNIHIAADSDMPDGITAGIVVANHQYEGDWWFMLMVARFLGLHGNVKIIVRE
GLKKIPLLGWLVRLVEYPFISSSWSLSRTNLFGLLRSFNADDFPVLLFQFPEGDRIDAKV
RQQSLAFAAKEQRPHLLHVLLPRTTGFNTCIEALRTSHPPVYDMTIAIPGTTGQPSSSSS
SSSPSAAAAAASPSAGAAAAAAAASNTSTNPSSNPPSSSSTDTSPAAAAAAAADAAAAES
HDASLFHTFLRFCNGEGPRDVHIRLKRYSLNDVLADPHWLDTKWAEKDRVLTYFSRHACF
PAPLPPHMANGGGSTQTHGGRGGPRGPAGSHAVVGAAGNAQYLRSFNSRKFKAETSFLAL
ARLLLTPLCLPLLLLMACPLMTLYVCVSTVRRWLGEEIVFSPGGREGGREGDAVYESVNW
SV
>LC107613.1 Nannochloropsis sp. NIES-2145 LPAT4 mRNA for
lysophosphatidate acyltransferase 4, complete cds
(SEQ ID NO: 47)
ATGCCGACCTATGCGGACCCTCCCAGGTCGCGCGCCTACCAGCACGAAAGCTCCTCCTACAGCATTGATA
ATCCGATCCGCAGCACCCACGCCTCCAGCCCACCCGACTATTTCAATCAAAACTCCCTCCGTGAGTCCCC
CTCCCTCCACTCCTCATCCTCAGAATCCGAGATGGACTCTCAGAGCAATGGCAATGGCAGGACCCCTCAA
CAGCGGCGAGGCGACATTCAAAGGCAGACAAACAACCCTTCCGCCTCCGGCACGAACGGCACCCCCCGAG
GAGCATTCAAGAGCACAATCTTCTCCATGGCCCACGCTGTCGTCCTCTGCTGGCTCGTGATGGCCATGTC
CTTCGGCATCAATATTTTGAATTTCCTCGTTTTTGTAACCGTCCGTCCTTGGTCCCGCCTGACGGCCCGA
AGGCTCATCGGTCACTGGTGGCAACGGATGTGGGTCTCAGTAATGAGCTACTTGCTCCCTAAGAGCGAGA
TGATTCTGACAGGGGACATCGTGCTTAACACTGTGGATTCCTCTCGCCCGGCCATCATCATCGCGAATCA
CCAGGTGGATGCGGATTTTTGGTATGTCTGGGAAGTGGCGCGGGCCTACGGAATGCATGGACGGTTGAAG
ATCATTTTGAAAGCCGATTTGGCTGTGGTCCCCATCGTGGGCTGGGGGATGAAACAATTTGAATTTTGTT
TTTTGCAACGCAATTGGCAGAGGGACCGGAGGGCGTTCACGCGCTTGTTGTCGTCGTTTGTCGAGGACGG
GTATAAGTGTGCCTTGCTCCTCTTTCCAGAGGGCACGACCATCAACACTGAAGCTGTCACCAAGTCCCAC
CGCTTCGGCCGTGAGCAGAAGAGGCCTCACCTCGACCATTGCATCCTACCGCGTTCCACGGGCTTTGCGG
CTTTGGTCGAGACAATGTGCCAGTCCCCCCATGGCCATTCTCCTGTTATTTACGATCTGACCGTCGCCTA
TCACGGTTACTCAGGCGAAGTTCCCACCTATGAGATGGGCTACGACCGAGAACAAGACATGGACGTCCCG
AACGTGTTCAAAATGATACAGGGACGGGCATGTCCAAAGGTTCATGTCCACGTCAAGACGTACGAAGTCG
ATGAGGCCTTGGTTAATAATCCCGAGCAGTGGTTGGATGCTCGATGGTTGGAAAAAGACGCGCTGCTGGA
ACGGTTTATTCAGACGCAGAGTTTTAGGGGGGAGAAGGAGGGAGGGACACGGATCATTCATCCTCAGGGG
AGCTTGGCCTCGCTGCTTTTTTTGTTGATGTTGCCGCTGATTATCACCGTCCTCCTTCCTGCCCTGCTCT
TGATCACTTTGGTGGCCTGGCCTGTGATTCTTCTGGCGGGCACGCTCAACTTTCTGAACTTTGTCTCGCG
TTCGGCCATGGGTGTGGTAGTCGGGAGCAGCAGCAGCCGTGGGTCTTTCTCCACGTCTTGGCATCAGTCC
TCCCAACACCGGCACCAGCACCAGCAACGGGTCAGTGGCAATAGCACCAGTAGCACCAGTACCAGCAACA
GTACCAGCAGTAGCATTGGTCGGAAGGAAGGGGCATTGAACTGCAGGCGAAGTTCTCGCCGAAGCCCCAG
CCGTGAGGGATGTGGCCACGCCAATGGAGAAGGACCGAGCTGTCCTCTCCTCTCTGTAAGCCTGCCGCCT
CTCCCTGCCGCAGCTGCTGCTGCCGCTCCTGCTGCTCCTGCTTCTTCGGCTCCATCTTCTCCGCTATTGG
GGAGCACGCCGTCGCCACGCCGGTCCCCACGGTGGAAGACTCCGGCGGGGATATCCCCTGTAGTGCAGAG
CTACGGGAACAGGAAGGGGAAGGAAGGAGGATGGGGTCTAATGGCTGCGGCGGCCGCAGCGGCAACGACG
GAAGAGGAGGATCGGTACTAG
>Nannochloropsis sp. NIES-2145 LPAT4 amino acid sequence
(SEQ ID NO: 48)
MPTYADPPRSRAYQHESSSYSIDNPIRSTHASSPPDYFNQNSLRESPSLHSSSSESEMDS
QSNGNGRTPQQRRGDIQRQTNNPSASGTNGTPRGAFKSTIFSMAHAVVLCWLVMAMSFGI
NILNFLVFVTVRPWSRLTARRLIGHWWQRMWVSVMSYLLPKSEMILTGDIVLNTVDSSRP
AIIIANHQVDADFWYVWEVARAYGMHGRLKIILKADLAVVPIVGWGMKQFEFCFLQRNWQ
RDRRAFTRLLSSFVEDGYKCALLLFPEGTTINTEAVTKSHRFGREQKRPHLDHCILPRST
GFAALVETMCQSPHGHSPVIYDLTVAYHGYSGEVPTYEMGYDREQDMDVPNVFKMIQGRA
CPKVHVHVKTYEVDEALVNNPEQWLDARWLEKDALLERFIQTQSFRGEKEGGTRIIHPQG
SLASLLFLLMLPLIITVLLPALLLITLVAWPVILLAGTLNFLNFVSRSAMGVVVGSSSSR
GSFSTSWHQSSQHRHQHQQRVSGNSTSSTSTSNSTSSSIGRKEGALNCRRSSRRSPSREG
CGHANGEGPSCPLLSVSLPPLPAAAAAAAPAAPASSAPSSPLLGSTPSPRRSPRWKTPAG
ISPVVQSYGNRKGKEGGWGLMAAAAAAATTEEEDRY
>BAA18602.1 sl11848 [Synechocystis sp. PCC 6803] mRNA
sequence
(SEQ ID NO: 49)
GTGGATTCCGAGATTAATCATCGTGGTGGTTTGAGTGCTCCCCGCCCAAGGGAAACGTCA
CTTAATTTAGCTCTCTACCGGGGCTTGAAATGGGGGGTGGTGCGGCCACTGCTCCATGGA
TTGTTCCAGGCCCAGGTATATGGTCAGGAATTGGTGCCAACCCGGGGGCCGGCCTTGGTG
GTGAGCAACCATGCCAGTTATTTTGACCCCCCATTTTTGTCCTGTGCCATGGCCCGGCCG
GTGGCCTTTATGGCCAAGGAAGAGTTATTTAATGTGCCCCTGCTGGGTCCAGCCATTCGC
CTCTATGGGGCCTATCCAGTCAAACGGGGCAGTGGCGATCGGGGAGCATTGCGGGCCGCC
TTGACGGCGCTGGGGGATGGTTGGTTAGTGGGGGTCTTTCTGGAGGGAACCAGAACAAAG
GATGGCCGCATTCACCAGCCAAAATTGGGGGCTGCCATGATTGCAGCTAAAGCCCAAGTG
CCCATTATTCCCGTCAGCCTAGGGGGAGTAGAGCAAATTTTTCAGCCCGGTTCCCCCTGG
CCCCATCCTGTGCCTTTAACTATTCGCATTGGTAAGGCGATCGCCCCTCCAGTAAAGAAT
AGGAAACCCGAATTGGAAGCGGTTACTAAAGCTTGCCAAGCCCAAATTCACGAGATGCTG
GATTTAGGCAGGGATTAG
>BAA18602.1 sl11848 [Synechocystis sp. PCC 6803] amino acid sequence
(SEQ ID NO: 50)
MDSEINHRGGLSAPRPRETSLNLALYRGLKWGVVRPLLHGLFQAQVYGQELVPTRGPALVVSNHASYFDP
PFLSCAMARPVAFMAKEELFNVPLLGPAIRLYGAYPVKRGSGDRGALRAALTALGDGWLVGVFLEGTRTK
DGRIHQPKLGAAMIAAKAQVPIIPVSLGGVEQIFQPGSPWPHPVPLTIRIGKAIAPPVKNRKPELEAVTK
ACQAQIHEMLDLGRD

Nucleotide Sequence Encoding ER LPAT2 of Arabidopsis thaliana

>ENA|AEE79683|AEE79683.1 Arabidopsis thaliana
(thale cress) lysophosphatidyl acyltransferase 2
(SEQ ID NO: 51)
ATGGTGATTGCTGCAGCTGTCATCGTGCCTTTGGGCCTTCTCTTCTTCAT
ATCTGGTCTCGCTGTCAATCTCTTTCAGGCAGTTTGCTATGTACTCATTC
GACCACTGTCTAAGAACACATACAGAAAAATTAACCGGGTGGTTGCAGAA
ACCTTGTGGTTGGAGCTTGTATGGATAGTTGACTGGTGGGCTGGAGTTAA
GATCCAAGTGTTTGCTGATAATGAGACCTTCAATCGAATGGGCAAAGAAC
ATGCTCTTGTCGTTTGTAATCACCGAAGTGATATTGATTGGCTTGTGGGA
TGGATTCTGGCTCAGCGGTCAGGTTGCCTGGGAAGCGCATTAGCTGTAAT
GAAGAAGTCTTCCAAATTCCTTCCAGTCATAGGCTGGTCAATGTGGTTCT
CGGAGTATCTCTTTCTGGAAAGAAATTGGGCCAAGGATGAAAGCACTCTA
AAGTCAGGTCTTCAGCGCTTGAGCGACTTCCCTCGACCTTTCTGGTTAGC
CCTTTTTGTGGAGGGAACTCGCTTTACAGAAGCCAAACTTAAAGCCGCAC
AAGAGTATGCAGCCTCCTCTGAATTGCCTATCCCTCGAAATGTGTTGATT
CCTCGCACCAAAGGTTTCGTGTCAGCTGTTAGTAATATGCGTTCATTTGT
CCCAGCAATTTATGATATGACAGTGACTATTCCAAAAACCTCTCCACCAC
CCACGATGCTAAGACTATTCAAAGGACAACCTTCAGTGGTGCATGTTCAC
ATCAAGTGTCACTCGATGAAAGACTTACCTGAATCAGATGACGCAATTGC
ACAGTGGTGCAGAGATCAGTTTGTGGCTAAGGATGCTCTGTTAGACAAAC
ACATAGCTGCAGACACTTTCCCCGGTCAACAAGAACAGAACATTGGCCGT
CCCATAAAGTCCCTTGCGGTGGTTCTATCATGGGCATGCGTACTAACTCT
TGGAGCAATAAAGTTCCTACACTGGGCACAACTCTTTTCTTCATGGAAAG
GTATCACGATATCGGCGCTTGGTCTAGGTATCATCACTCTCTGTATGCAG
ATCCTGATACGCTCGTCTCAGTCAGAGCGTTCGACCCCAGCCAAAGTCGT
CCCAGCCAAGCCAAAAGACAATCACCACCCAGAATCATCCTCCCAAACAG
AAACGGAGAAGGAGAAGTAA

Amino Acid Sequence for ER LPAT2 of Arabidopsis thaliana

>sp|Q8LG50|LPAT2_ARATH 1-acyl-sn-glycerol-3-
phosphate acyltransferase 2 OS = Arabidopsis
thaliana OX = 3702 GN = LPAT2 PE = 1 SV = 2
(SEQ ID NO: 52)
MVIAAAVIVPLGLLFFISGLAVNLFQAVCYVLIRPLSKNTYRKINRVVAE
TLWLELVWIVDWWAGVKIQVFADNETFNRMGKEHALVVCNHRSDIDWLVG
WILAQRSGCLGSALAVMKKSSKFLPVIGWSMWFSEYLFLERNWAKDESTL
KSGLQRLSDFPRPFWLALFVEGTRFTEAKLKAAQEYAASSELPIPRNVLI
PRTKGFVSAVSNMRSFVPAIYDMTVTIPKTSPPPTMLRLFKGQPSVVHVH
IKCHSMKDLPESDDAIAQWCRDQFVAKDALLDKHIAADTFPGQQEQNIGR
PIKSLAVVLSWACVLTLGAIKFLHWAQLFSSWKGITISALGLGIITLCMQ
ILIRSSQSERSTPAKVVPAKPKDNHHPESSSQTETEKEK

Nucleotide Sequence Encoding ER LPAT2 of Helianthus annuus

>ENA|OTG36509|OTG36509.1 Helianthus annuus
(common sunflower) putative 1-acyl-sn-
glycerol-3-phosphate acyltransferase 2
(SEQ ID NO: 53)
ATGGCTATCGCAGCAGCAGCTGTTATCGTCCCTATTGGCGTCCTCTTCTT
CGTCTCCGGCCTCATCGTCAATCTCATTCAGGCGATTATATTTGTGACTG
TACGACCGTTCTCGAAGAGCTTGTTTAGGCGGATTAACAGACAGGTAGCT
GAGTTGTTGTGGCTGGAGCTTGTGTGGATTGTTGATTGGTGGGCTGGAGT
TAAGGTTAACCTGTACACAGATGCCGAGACCCTGAAGATGATGGGTAAAG
AACATGCTCTTGTGATAGCTAATCATAAAAGTGACATTGATTGGCTCATT
GGATGGGTGTTTGCTCAGAGATCAGGTTGTCTTGGTAGCACATTGGCTGT
CATGAAGAAATCATCGAAGTTTCTTCCCGTCATTGGTTGGTCAATGTGGT
TTTCTGAGTATCTTTTTCTTGAGAGAAGTTGGGCTAAAGATGAAAGTACC
TTGAAGTCAGGCCTCCGACGTCTAAAAGATTACCCTCAACCCTTTTGGTT
GGCCCTTTTTGTTGAAGGGACTCGCTTTACTAAAGCAAAACTTTTAGCAG
CTCAAGAATATGCATCTTCAATGGGATTACCTGTTCCCAGAAATGTCTTA
ATTCCAAGAACAAAGGGATTTGTTACTTCAGTGAGTGAAATGAGATCATT
TGCTCCTGCAATTTACGATATGACGGTTGCGATTCCCAAAGATTCAACTC
CGCCAACAATGCTGCGCCTCTTTAAAGGGCAGTCGTCTGTGATTCACGTT
AAAGTTAAGCGTCATTTAATGAAGGACCTGCCAGAAACAGATGAAGGTGT
TGCACAATGGTGTAAAGATATTTTTGTTGCCAAGGATGATATATTAGATA
AACATAAAGAATTAAACGCCTTTCCTGATTCAGAACTCCATGAAATTGGC
CGACCATTGAAGTCTCTTGTGGTGGTTGTATCTTGGGCATGTCTGCTTGT
ACTCGGGATCTTCAAGTTCCTGCAATGGTCTAATCTTTTATCCTCATGGA
AGGGGCTCACATTCACTGCAATTGGGTTGGGGATTGTTACCTTTTTAATG
CAAATCTTGATTCAGTTTTCGCAATCTGAACGTTCTACACCTGCAAAAGT
GGCCCCCACAAGGTCTAGCAATGGTAATGTACAAGAGAAACTGCACTGA

Amino Acid Sequence for LPAT2 of Helianthus annuus

>tr|E6Y2I1|E6Y2I1_HELAN Putative 1-acyl-sn-
glycerol-3-phosphate acyltransferase 2
OS = Helianthus annuus OX = 4232
GN = LPAAT2 PE = 2 SV = 1
(SEQ ID NO: 54)
MAIAAAAVIVPIGVLFFVSGLIVNLIQAIIFVTVRPFSKSLFRRINRQVA
ELLWLELVWIVDWWAGVKVNLYTDAETLKMMGKEHALVIANHKSDIDWLI
GWVFAQRSGCLGSTLAVMKKSSKFLPVIGWSMWFSEYLFLERSWAKDEST
LKSGLRRLKDYPQPFWLALFVEGTRFTKAKLLAAQEYASSMGLPVPRNVL
IPRTKGFVTSVSEMRSFAPAIYDMTVAIPKDSTPPTMLRLFKGQSSVIHV
KVKRHLMKDLPETDEGVAQWCKDIFVAKDDILDKHKELNAFPDSELHEIG
RPLKSLVVVVSWACLLVLGIFKFLQWSNLLSSWKGLTFTAIGLGIVTFLM
QILIQFSQSERSTPAKVAPTRSSNGNVQEKLH

Nucleotide Sequence Encoding for ER LPAT2 of Camelina sativa

>XM_010429345.2 PREDICTED: Camelina sativa 1-
acyl-sn-glycerol-3-phosphate acyltransferase 2
(LOC104712441), mRNA
(SEQ ID NO: 55)
CTCCTTATAATTTCAATCGCTCCCATTTAATCGCAAAGTTATTTTTATTT
TATTTTTCTGGTTGGCTTATTTTTTATTTTATTTTATGTTTTAAATGTTC
TGCAACTTGTCTTGAAATCTGGGGTAAAAAGAAAAAAGAAAAAAAAAACT
TCGCTATTGGTTTTTCTGGAAATCTCAGAAACGATTATTTTGGGTTTTCT
ATACTTCGCTTCCTCACATTAGAGCTTCGGTGTTCTTTTTTTTCCTTTTC
TTTTTCTTTTTATTTGGGTGTGAGGGACATTTTTCCATGGTGATTGCTGC
AGCTGTCATCGTGCCTTTGGGCCTTCTCTTCTTCATATCTGGTCTCGTTG
TCAATCTCATTCAGGCACTTTGCTATGTCCTCATTCGGCCACTGTCTAAG
AACACTTACCGGAAAATCAACCGGGTGGTTGCTGAAACCTTGTGGTTGGA
GCTTGTTTGGATTGTTGATTGGTGGGCTGGAGTAAAGATCCAAGTGTTTG
CTGATAATGAGACCTTCAATCGAATGGGCAAAGAACACGCTCTTGTCGTT
TGTAATCACCGTAGTGATATTGATTGGCTTGTTGGATGGGTTCTGGCTCA
GCGGTCAGGTTCCCTGGGAAGCGCTTTGGCTGTAATGAAGAAGTCTTCCA
AATTCCTTCCAGTCATAGGCTGGTCAATGTGGTTCTCAGAGTATCTGTTT
CTGGAAAGAAATTGGTCCAAGGATGAAAGCACTCTAAAGTCAGGTCTTCA
GCGCTTGAGTGACTTTCCTCGACCTTTCTGGCTAGCCCTTTTTGTGGAGG
GAACTCGCTTTACAGAGGCTAAACTCAAAGCAGCACAAGAGTATGCAGCC
TCCTCTGACTTGCCTATCCCTCGAAATGTGTTGATTCCTCGCACCAAAGG
TTTTGTGTCAGCTGTTAGTAATATGCGTTCATTTGTCCCAGCCATTTATG
ATATGACAGTGACTATTCCAAAAACTTCTCCACCGCCCACGATGCTAAGA
CTATTCAAAGGACAACCTTCTGTGGTACATGTTCACATCAAGTGTCACTC
CATGAAAGACTTGCCTGAATCAGATGACGCAATTGCACAGTGGTGCAGAG
ATCAGTTTGTGGCTAAGGATGCTTTGTTAGACAAACACATAGCTGCAGAC
ACTTTCCCCGGTCAACAGGAACAGAACATTGGCCGTCCCATAAAGTCCCT
TGCGGTGGTTCTATCATGGGCATGCGTATTAACTCTTGGAGCAATTAAGT
TCCTACACTGGGCACAACTCTTTTCGTCATGGAAAGGTATCGCGCTATCG
GGGCTTGCTCTGGGTATCATCACTCTCGGTATGCAGATCCTGATACGCTC
GTCTCAGTCAGAGCGTTCAACCCCAGCCAAAGTGGTTCCAGCAAAGCCAA
AGGACCATCACAACTCAGAATCATCCTCCCAAACAGAAGTGGAGAAGCAG
AAGTAAAAAAAGTGGATATCAAAGATCAAACAACAAACAGAAGAAGAAAA
GCGTATCAGTTTTGTTA

Amino Acid Sequence of ER LPAT2 of Camelina sativa

>XP_010427647.1 PREDICTED: 1-acyl-sn-glycerol-3-
phosphate acyltransferase 2 [Camelina sativa]
(SEQ ID NO: 56)
MVIAAAVIVPLGLLFFISGLVVNLIQALCYVLIRPLSKNTYRKINRVVAE
TLWLELVWIVDWWAGVKIQVFADNETFNRMGKEHALVVCNHRSDIDWLVG
WVLAQRSGSLGSALAVMKKSSKFLPVIGWSMWFSEYLFLERNWSKDESTL
KSGLQRLSDFPRPFWLALFVEGTRFTEAKLKAAQEYAASSDLPIPRNVLI
PRTKGFVSAVSNMRSFVPAIYDMTVTIPKTSPPPTMLRLFKGQPSVVHVH
IKCHSMKDLPESDDAIAQWCRDQFVAKDALLDKHIAADTFPGQQEQNIGR
PIKSLAVVLSWACVLTLGAIKFLHWAQLFSSWKGIALSGLALGIITLGMQ
ILIRSSQSERSTPAKVVPAKPKDHHNSESSSQTEVEKQK
Nucleotide sequence encoding for ER LPAT2 of
Brassica napus:
>ENA|CAB09138|CAB09138.1 Brassica napus (rape)
acyl-CoA:1-acylglycerol-3-phosphate
acyltransferase
(SEQ ID NO: 57)
ATGGCGATGGCAGCAGCAGTGATTGTGCCTTTGGGGATTCTCTTCTTCAT
TTCTGGCCTCGTTGTCAATCTCCTCCAGGCAGTTTGCTATGTCCTCGTTC
GACCTATGTCTAAGAACACATACAGAAAGATCAACCGGGTGGTTGCAGAA
ACCTTGTGGTTGGAGCTTGTCTGGATCGTTGACTGGTGGGCTGGAGTCAA
GATCCAAGTCTTTGCTGATGATGAGACCTTTAATCGAATGGGCAAAGAAC
ATGCTCTTGTCGTTTGTAATCACCGAAGTGATATTGATTGGCTTGTTGGA
TGGATTCTCGCTCAGAGGTCAGGTTGCCTGGGAAGCGCATTAGCTGTAAT
GAAGAAGTCTTCCAAATTTCTCCCAGTCATAGGCTGGTCAATGTGGTTCT
CCGAGTATCTGTTTCTTGAAAGAAATTGGGCAAAGGATGAAAGCACTTTA
CAGTCAGGTCTTCAACGCTTGAACGACTTCCCACGGCCTTTCTGGCTAGC
TCTTTTTGTGGAGGGAACCCGCTTCACAGAGGCAAAACTTAAAGCAGCAC
AAGAGTACGCAGCCTCCTCTGAGTTGCCTGTCCCTCGAAATGTGTTGATT
CCTCGCACCAAAGGTTTTGTGTCAGCTGTTAGTAACATGCGTTCATTTGT
GCCAGCCATATATGATATGACCGTGGCTATTCCAAAAACTTCTCCACCCC
CAACGATGCTAAGACTATTCAAAGGACAACCTTCTGTGGTGCATGTTCAC
ATCAAGTGTCACTCGATGAAGGACTTGCCTGAACCAGAAGACGAAATTGC
ACAGTGGTGCAGAGATCAGTTTGTGGCTAAGGATGCACTGTTAGACAAAC
ACATAGCTGCAGACACTTTCCCCGGTCAGAAAGAACAGAACATTGGCCGT
CCCATAAAGTCTCTTGCAGTGGTTGTATCATGGGCATGCCTACTAACTCT
TGGAGCAATGAAGTTCTTACACTGGTCAAACCTCTTTTCTTCGTGGAAAG
GCATCGCATTATCAGCCTTTGGTCTAGGCATCATCACTCTCTGTATGCAG
ATCCTGATCCGCTCCTCTCAGTCGGAGCGTTCAACACCTGCCAAAGTCGC
TCCAGCCAAGCCAAAGGACAATCACCAGTCAGGACCATCCTCCCAAACAG
AAGTGGAGGAGAAGCAGAAGTAA

Amino Acid Sequence of ER LPAT2 of Brassica napus

>sp|Q9XFW4|LPAT2_BRANA 1-acyl-sn-glycerol-3-
phosphate acyltransferase 2 OS = Brassica napus
OX = 3708 GN = LPAT2 PE = 2 SV = 1
(SEQ ID NO: 58)
MAMAAAVIVPLGILFFISGLVVNLLQAVCYVLVRPMSKNTYRKINRVVAE
TLWLELVWIVDWWAGVKIQVFADDETFNRMGKEHALVVCNHRSDIDWLVG
WILAQRSGCLGSALAVMKKSSKFLPVIGWSMWFSEYLFLERNWAKDESTL
QSGLQRLNDFPRPFWLALFVEGTRFTEAKLKAAQEYAASSELPVPRNVLI
PRTKGFVSAVSNMRSFVPAIYDMTVAIPKTSPPPTMLRLFKGQPSVVHVH
IKCHSMKDLPEPEDEIAQWCRDQFVAKDALLDKHIAADTFPGQKEQNIGR
PIKSLAVVVSWACLLTLGAMKFLHWSNLFSSWKGIALSAFGLGIITLCMQ
ILIRSSQSERSTPAKVAPAKPKDNHQSGPSSQTEVEEKQK

### Nucleotide Sequence Encoding ER LPAT2 of Glycine max

>XM_003535809.4 Glycine max 1-acyl-sn-glycerol-3-
phosphate acyltransferase 2 (LOC100814107), mRNA
(SEQ ID NO: 59)
ATGACTGCAGTTGTGGTCGTCCCATTGGGCCTTCTCTTCTTCGCTTCCGG
CCTCATCGTTAACCTCATTCAGGCAATATGCTACGTGGTTGTGCGGCCGG
TGTCGAAGAATCTGTACCGGCGGATGAACAGGGTGGTGGCGGAGCTGCTC
TGGCTGGAGCTCGTGTGGATTATTGATTGGTGGGCCGGTGTTAAGGTTCA
AGTATTCACAGATCCTGAAACCTTTCGTTCAATGGGTAAAGAGCATGCTC
TTGTTATCTCCAATCACAGAAGTGACATTGATTGGCTTGTTGGATGGGTT
TTAGCTCAGCGTTCAGGTTGCCTTGGCAGCACTCTTGCTGTGATGAAGAA
ATCATCGAAGTTTCTACCGGTCATTGGTTGGTCAATGTGGTTTTCTGAAT
ATCTTTTCCTGGAGAGAAGTTGGGCCAAGGATGAACGCACATTAAAGTCA
GGCCTACAGCAACTGAGGGATTTCCCTCTTCCCTTTTGGTTGGCTCTCTT
TGTAGAAGGAACTCGCTTTACACAGGCCAAACTATTAGCTGCTCAGGAGT
ATGCAGCCTCAGCTGGATTGCCTGTTCCAAGAAATGTTTTGATTCCAAGA
ACTAAGGGTTTTGTTTCAGCAGTAAACCATATGCGCTCTTTTGTTCCTGC
CATTTATGATGTAACTGTGGCAATTCCCAAGAGTTCACCTGCTCCTACAA
TGCTAAGACTCTTCAGAGGGAAATCTTCAGTGGTTCATGTGCATATTAAG
CGGCATGCAATGAAGGATTTGCCAGAAGAAGATGAAGCTGTTGCTCAATG
GTGTCGAGATATGTTTGTGGCTAAGGATACATTGTTAGACAAACATATAG
CTGAGGACACATTCAGTGATCAAGAGCTGCAGGATACCGGTCGACCCATA
AAGTCTCTAGTGGTTGTCATATCGTGGGCATGTGTCGTTGTTATGGGGGT
CGTAAAGTTCCTCCAATGGTCATCACTACTATCCTCCTGGAAGGGTGTTG
CATTTTCAGCATTTGGTTTGGGAGTTGTTACTCTACTCATGCACATCTTG
ATCATGTTCTCACAATCTGAGCGTTCAACCCCTACCAAGGTTGCCCCTGC
AAAGTCCAAGAATAGCGAACAACTGGAGGCTAGGGATAACAAACAAGACT
AG

### Amino Acid Sequence for LPAT2 of Glycine max

>XP_003535857.1 1-acyl-sn-glycerol-3-phosphate
acyltransferase 2 [Glycine max]
(SEQ ID NO: 60)
MTAVVVVPLGLLFFASGLIVNLIQAICYVVVRPVSKNLYRRMNRVVAELL
WLELVWIIDWWAGVKVQVFTDPETFRSMGKEHALVISNHRSDIDWLVGWV
LAQRSGCLGSTLAVMKKSSKFLPVIGWSMWFSEYLFLERSWAKDERTLKS
GLQQLRDFPLPFWLALFVEGTRFTQAKLLAAQEYAASAGLPVPRNVLIPR
TKGFVSAVNHMRSFVPAIYDVTVAIPKSSPAPTMLRLFRGKSSVVHVHIK
RHAMKDLPEEDEAVAQWCRDMFVAKDTLLDKHIAEDTFSDQELQDTGRPI
KSLVVVISWACVVVMGVVKFLQWSSLLSSWKGVAFSAFGLGVVTLLMHIL
IMFSQSERSTPTKVAPAKSKNSEQLEARDNKQD

Nucleotide Sequence Encoding for ER LPAT of Yarrowia lipolytica

>ENA|CAG79722|CAG79722.1 Yarrowia lipolytica
CLIB122 YALI0E18964p:Location:1 . . . 849
(SEQ ID NO: 61)
ATGTCCGTTGCATCCAAGCTCGTCTTCTACGTCCGCGCCGCCATCGCCGT
GGTCATCTTTGCCGCCTGTGCCACCTACGGCGTGCTGGCGTCCACCATTC
TCACCGCCATCGGCAAGCAGGGCCTGGCCCAATGGACCGTTGCCAGAGCC
TTCTACTACTCGGTGCGCATCTTCCTGGGTATCAGCATCAAGCTGCGTAG
CCGGCAGGTGACCGGAACCGCCGGTCTGGATGCCTCCAAGATCCAGGTCG
CCAACACCACCAAGCCCATTGACGACATCACCAAACACCTGCCCCGACCA
TGCATTCTGATTTCCAACCACCAGAACGAAATGGACATTCTGGTGCTCGG
TCGCATCTTCCCCCAGTACTGCTCCGTCACCGCCAAAAAGGCCCTCAAGT
GGTACCCTCTGCTGGGCCAGTTCATGGCGCTGTCCGGCACCATCTTCCTG
GACCGAAAGGACCGAACCAAGTCCGTGCAGACCCTCGGCGGCGCCGTCAA
GACCATCCAGAGCGGCAACGGAGGCAAGGGCCAGAGCGTCTTCATGTTCC
CCGAGGGAACCCGATCCTACTCCAAGGACGTCGGCATCATGCCCTTCAAG
AAGGGCTGTTTCCACCTGGCGGTCCAGTCGGGCGCTCCCATTGTCCCCGT
GGTGGTCCAGAACACCTCCCGAATGTTTTCTTTCGGCCGAGGCAAGCTGG
ACGCCGGAGAGATCCTTGTCGACGTCCTGAGCCCCATTGAGACCAAGGGT
CTGGACGCCAGCAACGTCGACGCTCTCATGGCCACCACTTATAAGGCCAT
GTGCGAGACTGCCGACCAGATTGGCTACGCTGGCCAGAAGACTCAGTAG

Amino Acid Sequence for ER LPAT of Yarrowia lipolytica

>tr|Q6C5D5|Q6C5D5_YARLI 1-acyl-sn-glycerol-3-
phosphate acyltransferase OS=Yarrowia lipolytica
(strain CLIB 122/E 150) OX = 284591 GN = YALI0_
E18964g PE = 3 SV = 1
(SEQ ID NO: 62)
MSVASKLVFYVRAAIAVVIFAACATYGVLASTILTAIGKQGLAQWTVARA
FYYSVRIFLGISIKLRSRQVTGTAGLDASKIQVANTTKPIDDITKHLPRP
CILISNHQNEMDILVLGRIFPQYCSVTAKKALKWYPLLGQFMALSGTIFL
DRKDRTKSVQTLGGAVKTIQSGNGGKGQSVFMFPEGTRSYSKDVGIMPFK
KGCFHLAVQSGAPIVPVVVQNTSRMFSFGRGKLDAGEILVDVLSPIETKG
LDASNVDALMATTYKAMCETADQIGYAGQKTQ

Nucleotide Sequence Encoding for PDCT of Arabidopsis thaliana

>ENA|AEE75730|AEE75730.1 Arabidopsis thaliana
(thale cress) phosphatidic acid phosphatase-
related/PAP2-like protein
(SEQ ID NO: 63)
ATGTCAGCCGCCGCAGCTGAAACCGACGTCTCTCTCCGTCGCAGATCTAA
CTCTCTTAACGGAAACCACACTAACGGCGTCGCCATTGACGGAACCCTAG
ACAACAACAACCGTCGCGTCGGAGATACAAACACTCACATGGATATATCT
GCTAAGAAAACTGACAACGGCTACGCCAATGGTGTCGGAGGAGGAGGATG
GAGAAGCAAAGCGTCGTTCACGACGTGGACGGCGCGTGATATCGTCTACG
TGGTGAGATACCATTGGATACCGTGCATGTTCGCTGCCGGACTTCTGTTC
TTCATGGGCGTGGAGTACACGCTTCAGATGATTCCCGCGAGATCTGAGCC
GTTCGATCTTGGGTTTGTGGTCACGCGCTCTTTGAATCGCGTATTAGCAT
CTTCACCGGATCTTAACACTGTTTTAGCCGCACTAAACACGGTGTTCGTA
GGGATGCAAACAACGTATATTGTATGGACATGGTTAGTGGAAGGACGAGC
ACGAGCCACCATCGCGGCTTTATTCATGTTCACTTGTCGCGGCATTCTCG
GCTACTCTACTCAGCTTCCTCTCCCTCAGGACTTTCTAGGATCAGGGGTT
GATTTTCCGGTGGGAAATGTCTCTTTCTTCCTCTTCTTCTCTGGCCATGT
CGCCGGCTCGATGATCGCATCATTGGACATGAGAAGAATGCAGAGGTTGA
GACTTGCAATGGTCTTTGACATCCTCAATGTATTACAGTCGATCAGACTG
CTCGGTACAAGAGGACATTACACAATCGACCTTGCGGTTGGAGTTGGCGC
TGGGATTCTCTTCGACTCATTGGCCGGAAAGTACGAAGAGATGATGAGCA
AGAGACATTTAGGCACTGGTTTTAGTTTGATTTCGAAAGACTCTCTAGTC
AATTAA

Amino Acid Sequence for PDCT of Arabidopsis thaliana

>sp/Q9LVZ7|PDCT1_ARATH Phosphatidylcholine:diacyl-
glycerol cholinephosphotransferase 1 OS =
Arabidopsis thaliana OX = 3702 GN = ROD1 PE = 1
SV=1
(SEQ ID NO: 64)
MSAAAAETDVSLRRRSNSLNGNHTNGVAIDGTLDNNNRRVGDTNTHMDIS
AKKTDNGYANGVGGGGWRSKASFTTWTARDIVYVVRYHWIPCMFAAGLLF
FMGVEYTLQMIPARSEPFDLGFVVTRSLNRVLASSPDLNTVLAALNTVFV
GMQTTYIVWTWLVEGRARATIAALFMFTCRGILGYSTQLPLPQDFLGSGV
DFPVGNVSFFLFFSGHVAGSMIASLDMRRMQRLRLAMVFDILNVLQSIRL
LGTRGHYTIDLAVGVGAGILFDSLAGKYEEMMSKRHLGTGFSLISKDSLV
N

Nucleotide Sequence encoding for PDCT of Helianthus annuus

>ENA|ARQ87993|ARQ87993.1 Helianthus annuus (common
sunflower) phosphatidylcholine: Location:
1 . . . 840
(SEQ ID NO: 65)
ATGGGGACAATCACAATGAACGCCGATAAGATCCACCAACGTTCCATACC
ACAAACAACCACCCTCCTCAAAACATCAAACAACGTTTCCATTAACAATT
TCAATCTCAAAAAGACCAACAATTCTACGACCACATGGGGATCGCTAGCG
GACGCGTCGTTTTTACAATGGACAACATCGGATGTGTTCGGTTTGTTTAA
GTACCACCCCGTCCCGTGTTTCTTTGCTGTTTCGTTACTGTTTTTTATGG
GTGTGGAGTATACGCTGCGGATGATACCGCCCTCGTCACCCCCGTTTGAT
ATTGGGTTCGTTGCCACTGCGTATTTGCATCGTGTCCTCGTCGCTAGTCC
GACGCTAAACACAGTTCTCGCAGGGCTTAATACGGTGTTTGTGGGGATGC
AAACAACCTATATAATAGGGACGTGGGTCATTGAAGGGCGGCCGAGAGCC
ACAGTCGCGGCGCTGTTCATGTTTACTTGCAGAGGGATTTTGGGTTATTC
GACACAGCTTCCTTTGCCTGAGGGATTCTTGGGTTCGGGAGCAGACTTTC
CGGTAGGCAATGTGTCCTTCTTCTTGTTTTATTCTGGTCATGTAGCAGGA
TCAGTAATTGCATCGTTTGATATGAAGAGAATGCATAGGTGGGAGTTGGC
CTTTCTATTCGACACCTTGAACCTTTTGCAGGTGGTGCGGTTACTAAGCA
CAAGGGGACACTACACCATTGACTTAGCCGTCGGAGTAGGTGCCGGCATG
TTGTTTGATTCCCTCGCCGGAAAATACATGAAGAAGGCTATGGCTGAAGG
AGGTGATGTCTTGTATACAAACCTACCTCACTTATCTTAA

Amino Acid Sequence for PDCT of Helianthus annuus

>tr|A0A3S5FWJ3|A0A3S5FWJ3_HELAN Phosphatidyl-
choline OS = Helianthus annuus OX = 4232 GN = PDCT
PE = 2 SV = 1
(SEQ ID NO: 66)
MGTITMNADKIHQRSIPQTTTLLKTSNNVSINNFNLKKTNNSTTTWGSLA
DASFLQWTTSDVFGLFKYHPVPCFFAVSLLFFMGVEYTLRMIPPSSPPFD
IGFVATAYLHRVLVASPTLNTVLAGLNTVFVGMQTTYIIGTWVIEGRPRA
TVAALFMFTCRGILGYSTQLPLPEGFLGSGADFPVGNVSFFLFYSGHVAG
SVIASFDMKRMHRWELAFLFDTLNLLQVVRLLSTRGHYTIDLAVGVGAGM
LFDSLAGKYMKKAMAEGGDVLYTNLPHLS

Nucleotide Sequence Encoding for PDCT of Camelina sativa

>XM_010467272.2 PREDICTED: Camelina sativa phos-
phatidylcholine: diacylglycerol cholinephospho-
transferase 1 (LOC104745897), mRNA
(SEQ ID NO: 67)
CACCAAAGAGTTAGAGTGTATTTAACTAACAACCATAAATTAAAAATGTC
CTTCTCAAAAATAACTATTCATCACTTTCATATGACAATTTTCTAACCCA
AACAAGTCTCCTTTGAAATCTCCGCCCTCGTATCTCTTATATAACACATC
TCTCTTCAAACTCCCAAAAATGTCCCTCTCCGTTAACTCTAACGGCCATG
TCAGTCGCCGCAGCTAAACCCGCCGTCTCTCGCCGTCACGTATCTAACGG
AAACAACACTAACAACGTCGCCATTGACGACGATCACAACCACCAACGCC
GCATCGTCGGAGATAAAAACACTCGAATGGAGATCGCTGCTAAGAACAAC
GGCTACGCCAACGGTGTCATCGGAGGAGGAGGATGGAGGAGCAAGGCGTC
GTTCATGACGTGGACGACGCGTGACGTTGTCTACGTGGCGAGACACCATT
GGATACCGTGCATGTTCGCTGCCGGGCTTTTGTTCTTCATGGGGGTCGAG
TACACGCTCCAGATGATTCCCGCGAGATCTGAGCCGTTCGATCTTGGGTT
CGTGGCCACGCGCTCTTTGAATCGCGTCTTAGCATCTTCCCCGGATCTTA
ACACCGTTTTAGCCGCACTAAACACGGTGTTCGTATTGATGCAAACAACG
TATATTGTATGGACATGGTTAGTGGAAGGACGAGCACGAGCAACCATCTC
GGCTTTATTCATGTTCACGTGTCGGGGCATTCTCGGCTACTCTACTCAGC
TTCCTCTCCCTCAGGATTTTTTAGGATCAGGAGTTGATTTTCCAGTGGGA
AACGTCTCTTTCTTCCTCTTCTTCTCGGGCCACGTTGCCGGCTCGATGAT
CGCATCACTGGACATGAGGAGAATGCAGAGGTTTAAGCTGGCGAGGGTTT
TTGACATCCTCAATGTATTACAATCGATCAGGCTGCTCGGTACAAGAGGA
CACTACACCATCGACCTTGCGGTTGGAGTTGGCGCTGGGATTCTCTTTGA
CTCACTGGCCGGGAAGTACGAAGAGATGAGCAGAAGACACCACCTAGGAA
CTGGTTTTAGTTTGATATCGAAAGACTCTCTAGTCAATTAAATTTGTTTT
CTATCAAATGTTTTTAGTTCAACACATTTAGTTAAGTTGAATTTAGTCTA
ATGACTTATTTATTTCTTCTTTATAAATGCCTAAATGGTTCATTGTATAC
CGTTTGTTGAACCTAATTATCGATCTAATGTTACTTAAAGGGTTGTAGCC
TTGTAGTTGTA

Amino Acid Sequence for PDCT of Camelina sativa

>XP_010465574.1 PREDICTED: phosphatidylcholine:
diacylglycerol cholinephosphotransferase 1
[Camelina sativa]
(SEQ ID NO: 68)
MSVAAAKPAVSRRHVSNGNNTNNVAIDDDHNHQRRIVGDKNTRMEIAAKN
NGYANGVIGGGGWRSKASFMTWTTRDVVYVARHHWIPCMFAAGLLFFMGV
EYTLQMIPARSEPFDLGFVATRSLNRVLASSPDLNTVLAALNTVFVLMQT
TYIVWTWLVEGRARATISALFMFTCRGILGYSTQLPLPQDFLGSGVDFPV
GNVSFFLFFSGHVAGSMIASLDMRRMQRFKLARVFDILNVLQSIRLLGTR
GHYTIDLAVGVGAGILFDSLAGKYEEMSRRHHLGTGFSLISKDSLVN

Nucleotide Sequence Encoding for PDCT of Brassica napus

>XM_013791764.2 PREDICTED: Brassica napus phospha-
tidylcholine: diacylglycerol cholinephospho-
transferase 1 (LOC106352083), mRNA
(SEQ ID NO: 69)
ATGAACTTTTGTAGCCCAAACAACCTTCCTTTCCTTCCACAAGTTTCATA
ATATCTCTTATATAACCCATATCTCCAAGCCTCTCGAAATGTTCTTCTCC
GTTAAATCTAACGGTCATGTCAACTACAACAATCGTCCCTCTCCGTCGCA
GTTCTAACTCTCTCAATGAATACCACACTAACGCAGTCGCCTTTGACGGA
ATCGTCGGGTCAACAAGTACTAGCCAAATGGAGGAGATTGTTACGCAAAC
CGACGACTGCTACGCCAATCACAACGGAGATGGAGGGAGAAGCAAGGCAT
CGTTTATGACGTGGAGGATGTGCAATCCTGTCCAGGTGGCGAGAGTCCAT
TGGATACCGTGTTTGCTAGCGGTAGGAGTTCTGTTCTTCACGGGCGTAGA
GGAGTACATGCTCCAGATGATTCCGGCGAGTTCTGAGCCGTTCGATATTG
GTTTTGTGGCGACGCGCTCTCTGTATCGACTCTTGGCTTCTTCACCGGAT
CTTAATACCGTTTTAGCTGCTCTCAACACGGTGTTTGTAGGGATGCAAAC
GACGTATATTTTATGGACATGGTTGGTGGAAGGACGACCACGAGCGACCA
TCTCGGCTTGCTTCATGTTTACTTGTCGTGGCATTCTTGGTTACTCTACT
CAGCTCCCTCTTCCTCAGGATTTTCTAGGATCAGGGGTAGATTTTCCGGT
AGGAAACGTCTCGTTCTTCCTCTTCTACTCAGGCCATGTCGCAGGGTCGA
CGATAGCATCCTTGGATATGAGGAGAATGAAGAGGTTGAGACTTGCCTTG
CTTTTTGACATCCTCAATGTATTACAATCGATCAGGCTTCTCGGGACGAG
AGGACAATACACGATCGATCTCGCTGTCGGAGTTGGCGCTGGGGTTCTCT
TTGACTCACTGGCTGGAAAATACGAAGAGATGATGAGCAAGAGACGCAAT
GTAGGCAATGGTTTTAGTTTGATTTCGTCTCGCTAGTTATTAATTTTTGT
TTTTTTTTTTTTATGTTTTTAGTCTGGACATATTTAATTTAGTTGAAATC
TAATGACTTAAATTTGCTTTCTTTCAAAATGCTCTAACTGACGGACCTAA
CTAAATGTGTACGTTATTGTGTAGTTACCATAGAGGTTTCGTATTGTCTT
GAGCCTGATATTTTGATTTTAGAGCTCGTTTATACGGTAGCTAATAATAA
AAAA

Amino Acid Sequence for PDCT of Brassica napu s

>XP_013647218.1 phosphatidylcholine: diacylgly-
cerol cholinephosphotransferase 1 [Brassica napus]
(SEQ ID NO: 70)
MSTTTIVPLRRSSNSLNEYHTNAVAFDGIVGSTSTSQMEEIVTQTDDCYA
NHNGDGGRSKASFMTWRMCNPVQVARVHWIPCLLAVGVLFFTGVEEYMLQ
MIPASSEPEDIGFVATRSLYRLLASSPDLNTVLAALNTVFVGMQTTYILW
TWLVEGRPRATISACFMFTCRGILGYSTQLPLPQDFLGSGVDFPVGNVSF
FLFYSGHVAGSTIASLDMRRMKRLRLALLFDILNVLQSIRLLGTRGQYTI
DLAVGVGAGVLFDSLAGKYEEMMSKRRNVGNGFSLISSR

### Nucleotide Sequence Encoding for PDCT of Glycine max

>XM_003531670.4 PREDICTED: Glycine max phosphati-
dylcholine: diacylglycerol cholinephosphotrans-
ferase 1 (LOC100790665), mRNA
(SEQ ID NO: 71)
ATGAATGGCGGCGCTGAGGCCTCCCTCAATCACAGGCGCAAACACCAAAC
AGCTCCCGCCGACGGCGCTAAAGGCGTTAAGGTAGCAAACGGAGCCATGG
GGAAGCCGTCCTCTTCCAAGCACTCCTGCGGCGCGTCGTTCATGAAATGG
ACCGTGGCTGACGCTGTCCACGTGGTGACGCACCATTGGATGCCGTGCTT
GTTCGCATTGGGGCTTCTCTTCTTCATGGCCGTGGAGTACACGCTTCTCA
TGGTGCCGCCGTCGTCGCCGCCCTTCGACCTTGGCTTCATCGCCACACGC
TCCCTCCACGCGCTCCTCGAGTCGTCGCCGAATCTCAACACGCTCTTCGC
CGGGCTCAATACGGTGTTTGTGGGGATGCAAACGAGTTATATCTTATGGA
CGTGGCTGATTGAAGGACGCCCCAGAGCCACAATTTCAGCATTGTTCATG
TTCACATGCCGTGGGATTTTAGGCTACTCCACCCAGCTCCCATTGCCTCA
GGGGTTTTTGGGCTCGGGTGTGGACTTCCCTGTTGGGAACGTGTCTTTTT
TCTTGTTTTTTTCTGGGCACGTTGCAGGGTCAGTGATTGCTTCATTGGAC
ATGAGGAGGATGCAGAGGTGGGAACTGGCTTGGACTTTTGATGTGCTCAA
TGTTTTGCAAGCTGTGAGGTTGCTGGGTACAAGAGGCCATTACACTATTG
ATTTGGCCGTAGGGGTTGGTGCTGGAATTCTCTTTGATTCTTTAGCTGGC
AAGTACGAAGATAGCAAAAGGAATGGTGCTCTCAAACACAATTTGATTGC
GTGA

### Amino Acid Sequence for PDCT of Glycine max

>XP_003531718.1 phosphatidylcholine: diacylgly-
cerol cholinephosphotransferase 1 [Glycine max]
(SEQ ID NO: 72)
MNGGAEASLNHRRKHQTAPADGAKGVKVANGAMGKPSSSKHSCGASFMKW
TVADAVHVVTHHWMPCLFALGLLFFMAVEYTLLMVPPSSPPFDLGFIATR
SLHALLESSPNLNTLFAGLNTVFVGMQTSYILWTWLIEGRPRATISALFM
FTCRGILGYSTQLPLPQGFLGSGVDFPVGNVSFFLFFSGHVAGSVIASLD
MRRMQRWELAWTFDVLNVLQAVRLLGTRGHYTIDLAVGVGAGILFDSLAG
KYEDSKRNGALKHNLIA

Arabidopsis thaliana At1g74960 (FAB1) Nucleotide Sequence

(SEQ ID NO: 78)
ATGGTGGGTGCGTCTTCCTCTTACGCATCTCCGTTATGTACCTGGTTTGT
TGCTGCTTGCATGTCCGTCTCTCACGGTGGAGGAGATAGCCGTCAGGCTG
TTGCTCTTCAATCTGGTGGGCGGAGTCGGCGAAGGAGGCAGCTTAGCAAA
TGCTCTGTCGCTTCTGGATCCGCTAGCATTCAGGCTCTCGTCACTTCTTG
TTTGGATTTTGGTCCTTGTACTCACTACAACAACAACAATGCATTGTCTT
CTCTCTTTGGATCGAATAGTGTTTCTTTGAATCGAAACCAGAGGAGATTG
AATCGTGCTGCTAGCTCCGGTGGAGCCATGGCAGTGATGGAGATGGAAAA
GGAAGCTGCGGTTAACAAGAAACCACCTACGGAGCAGCGTCGAGTTGTAG
TGACAGGCATGGGAGTTGAAACATCATTGGGTCATGACCCACATACCTTC
TATGAGAATTTGCTACAAGGCAACAGTGGTATTAGCCAGATTGAAAATTT
TGATTGTTCTGAATTTCCTACGCGAATTGCGGGAGAGATCAAAAGCTTCT
CGACTGAAGGATGGGTTGCTCCAAAACTTTCTAAAAGGATGGACAAATTC
ATGCTCTATCTTCTCACAGCTGGTAAGAAAGCTTTGGCTGATGGTGGGGT
TACTGATGAAGTAATGGCAGAGTTTGACAAAACCAAATGTGGAGTTTTGA
TTGGCTCGGCAATGGGAGGAATGAAGGTCTTTTACGATGCTATTGAAGCT
CTGAGAATCTCTTACAAGAAGATGAATCCTTTTTGTGTACCTTTTGCGAC
AACAAACATGGGTTCTGCTATGCTTGCCATGGATCTGGGATGGATGGGGC
CAAACTATTCTATTTCAACTGCTTGTGCCACAAGCAACTTTTGCATTCTG
AATTCAGCAAACCACATTATTAAAGGTGAAGCTGATGTAATGCTCTGTGG
TGGCTCAGATGCAGTTATTATTCCAATAGGGTTGGGAGGTTTTGTTGCAT
GCCGGGCTCTTTCACAAAGGAATAATGATCCCACAAAAGCTTCACGTCCT
TGGGATACCAATCGAGATGGTTTCGTGATGGGAGAGGGAGCTGGAGTTCT
ACTTTTGGAAGAACTCGAGCATGCTAAGAAAAGAGGTGCAACTATCTACG
CAGAGTTCCTCGGTGGGAGTTTCACATGTGATGCCTATCACATGACCGAG
CCTCACCCTGATGGGGCTGGTGTTATTCTCTGTATTGAGAGAGCGTTAGC
TAGTGCTGGGATTTCCAAGGAACAAATAAATTACATAAATGCACATGCAA
CCTCAACGCATGCTGGAGATATTAAGGAATACCAAGCCCTTGCTCACTGT
TTTGGCCAAAATCCTGAGCTTAAGGTAAATTCCACAAAATCTATGATTGG
ACACTTGCTGGGAGCTGCTGGGGCCGTGGAGGCTGTTGCAACTGTGCAGG
CGATACGGACCGGATGGGTTCATCCAAATATCAACCTCGAGAATCCAGAC
AGTGGAGTGGATACAAAGCTGCTGGTGGGTCCTAAGAAGGAGAGACTGGA
CATTAAAGCAGCCTTGTCAAATTCATTCGGGTTTGGTGGTCATAACTCCA
GCATCATTTTTGCTCCTTACAAGTGA

Arabidopsis thaliana At1g74960 (FAB1) Amino Acid Sequence

(SEQ ID NO: 79)
MVGASSSYASPLCTWFVAACMSVSHGGGDSRQAVALQSGGRSRRRRQLSK
CSVASGSASIQALVTSCLDFGPCTHYNNNNALSSLFGSNSVSLNRNQRRL
NRAASSGGAMAVMEMEKEAAVNKKPPTEQRRVVVTGMGVETSLGHDPHTF
YENLLQGNSGISQIENFDCSEFPTRIAGEIKSFSTEGWVAPKLSKRMDKF
MLYLLTAGKKALADGGVTDEVMAEFDKTKCGVLIGSAMGGMKVFYDAIEA
LRISYKKMNPFCVPFATTNMGSAMLAMDLGWMGPNYSISTACATSNFCIL
NSANHIIKGEADVMLCGGSDAVIIPIGLGGFVACRALSQRNNDPTKASRP
WDTNRDGFVMGEGAGVLLLEELEHAKKRGATIYAEFLGGSFTCDAYHMTE
PHPDGAGVILCIERALASAGISKEQINYINAHATSTHAGDIKEYQALAHC
FGQNPELKVNSTKSMIGHLLGAAGAVEAVATVQAIRTGWVHPNINLENPD
SGVDTKLLVGPKKERLDIKAALSNSFGFGGHNSSIIFAPYK

Arabidopsis thaliana At3g12120 (FAD2) Nucleotide Sequence

(SEQ ID NO: 80)
ATGGGTGCAGGTGGAAGAATGCCGGTTCCTACTTCTTCCAAGAAATCGGA
AACCGACACCACAAAGCGTGTGCCGTGCGAGAAACCGCCTTTCTCGGTGG
GAGATCTGAAGAAAGCAATCCCGCCGCATTGTTTCAAACGCTCAATCCCT
CGCTCTTTCTCCTACCTTATCAGTGACATCATTATAGCCTCATGCTTCTA
CTACGTCGCCACCAATTACTTCTCTCTCCTCCCTCAGCCTCTCTCTTACT
TGGCTTGGCCACTCTATTGGGCCTGTCAAGGCTGTGTCCTAACTGGTATC
TGGGTCATAGCCCACGAATGCGGTCACCACGCATTCAGCGACTACCAATG
GCTGGATGACACAGTTGGTCTTATCTTCCATTCCTTCCTCCTCGTCCCTT
ACTTCTCCTGGAAGTATAGTCATCGCCGTCACCATTCCAACACTGGATCC
CTCGAAAGAGATGAAGTATTTGTCCCAAAGCAGAAATCAGCAATCAAGTG
GTACGGGAAATACCTCAACAACCCTCTTGGACGCATCATGATGTTAACCG
TCCAGTTTGTCCTCGGGTGGCCCTTGTACTTAGCCTTTAACGTCTCTGGC
AGACCGTATGACGGGTTCGCTTGCCATTTCTTCCCCAACGCTCCCATCTA
CAATGACCGAGAACGCCTCCAGATATACCTCTCTGATGCGGGTATTCTAG
CCGTCTGTTTTGGTCTTTACCGTTACGCTGCTGCACAAGGGATGGCCTCG
ATGATCTGCCTCTACGGAGTACCGCTTCTGATAGTGAATGCGTTCCTCGT
CTTGATCACTTACTTGCAGCACACTCATCCCTCGTTGCCTCACTACGATT
CATCAGAGTGGGACTGGCTCAGGGGAGCTTTGGCTACCGTAGACAGAGAC
TACGGAATCTTGAACAAGGTGTTCCACAACATTACAGACACACACGTGGC
TCATCACCTGTTCTCGACAATGCCGCATTATAACGCAATGGAAGCTACAA
AGGCGATAAAGCCAATTCTGGGAGACTATTACCAGTTCGATGGAACACCG
TGGTATGTAGCGATGTATAGGGAGGCAAAGGAGTGTATCTATGTAGAACC
GGACAGGGAAGGTGACAAGAAAGGTGTGTACTGGTACAACAATAAGTTAT
GA

Arabidopsis thaliana At3g12120 (FAD2) Amino Acid Sequence

(SEQ ID NO: 81)
MGAGGRMPVPTSSKKSETDTTKRVPCEKPPFSVGDLKKAIPPHCFKRSIP
RSFSYLISDIIIASCFYYVATNYFSLLPQPLSYLAWPLYWACQGCVLTGI
WVIAHECGHHAFSDYQWLDDTVGLIFHSFLLVPYFSWKYSHRRHHSNTGS
LERDEVFVPKQKSAIKWYGKYLNNPLGRIMMLTVQFVLGWPLYLAFNVSG
RPYDGFACHFFPNAPIYNDRERLQIYLSDAGILAVCFGLYRYAAAQGMAS
MICLYGVPLLIVNAFLVLITYLQHTHPSLPHYDSSEWDWLRGALATVDRD
YGILNKVFHNITDTHVAHHLFSTMPHYNAMEATKAIKPILGDYYQFDGTP
WYVAMYREAKECIYVEPDREGDKKGVYWYNNKL

Arabidopsis thaliana At4g34520 (FAE1) Nucleotide Sequence

(SEQ ID NO: 82)
ATGACGTCCGTTAACGTTAAGCTCCTTTACCGTTACGTCTTAACCAACTT
TTTCAACCTCTGTTTGTTCCCGTTAACGGCGTTCCTCGCCGGAAAAGCCT
CTCGGCTTACCATAAACGATCTCCACAACTTCCTTTCCTATCTCCAACAC
AACCTTATAACAGTAACTTTACTCTTTGCTTTCACTGTTTTCGGTTTGGT
TCTCTACATCGTAACCCGACCCAATCCGGTTTATCTCGTTGACTACTCGT
GTTACCTTCCACCACCGCATCTCAAAGTTAGTGTCTCTAAAGTCATGGAT
ATTTTCTACCAAATAAGAAAAGCTGATACTTCTTCACGGAACGTGGCATG
TGATGATCCGTCCTCGCTCGATTTCCTGAGGAAGATTCAAGAGCGTTCAG
GTCTAGGTGATGAGACGTACAGTCCTGAGGGACTCATTCACGTACCACCG
CGGAAGACTTTTGCAGCGTCACGTGAAGAGACAGAGAAGGTTATCATCGG
TGCGCTCGAAAATCTATTCGAGAACACCAAAGTTAACCCTAGAGAGATTG
GTATACTTGTGGTGAACTCAAGCATGTTTAATCCAACTCCTTCGCTATCC
GCTATGGTCGTTAATACTTTCAAGCTCCGAAGCAACATCAAAAGCTTTAA
TCTAGGAGGAATGGGTTGTAGTGCTGGTGTTATTGCCATTGATTTGGCTA
AAGACTTGTTGCATGTTCATAAAAACACTTATGCTCTTGTGGTGAGCACT
GAGAACATCACACAAGGCATTTATGCTGGAGAAAATAGATCAATGATGGT
TAGCAATTGCTTGTTTCGTGTTGGTGGGGCCGCGATTTTGCTCTCTAACA
AGTCGGGAGACCGGAGACGGTCCAAGTACAAGCTAGTTCACACGGTCCGA
ACGCATACTGGAGCTGATGACAAGTCTTTTCGATGTGTGCAACAAGAAGA
CGATGAGAGCGGCAAAATCGGAGTTTGTCTGTCAAAGGACATAACCAATG
TTGCGGGGACAACACTTACGAAAAATATAGCAACATTGGGTCCGTTGATT
CTTCCTTTAAGCGAAAAGTTTCTTTTTTTCGCTACCTTCGTCGCCAAGAA
ACTTCTAAAGGATAAAATCAAGCATTACTATGTTCCGGATTTCAAGCTTG
CTGTTGACCATTTCTGTATTCATGCCGGAGGCAGAGCCGTGATCGATGAG
CTAGAGAAGAACTTAGGACTATCGCCGATCGATGTGGAGGCATCTAGATC
AACGTTACATAGATTTGGGAATACTTCATCTAGCTCAATTTGGTATGAAT
TAGCATACATAGAGGCAAAGGGAAGAATGAAGAAAGGGAATAAAGCTTGG
CAGATTGCTTTAGGATCAGGGTTTAAGTGTAATAGTGCGGTTTGGGTGGC
TCTACGCAATGTCAAGGCATCGGCAAATAGTCCTTGGCAACATTGCATCG
ATAGATATCCGGTTAAAATTGATTCTGATTTGTCAAAGTCAAAGACTCAT
GTCCAAAACGGTCGGTCCTAA

Arabidopsis thaliana At4g34520 (FAE1) Amino Acid Sequence

(SEQ ID NO: 83)
MTSVNVKLLYRYVLTNFFNLCLFPLTAFLAGKASRLTINDLHNFLSYLQH
NLITVTLLFAFTVFGLVLYIVTRPNPVYLVDYSCYLPPPHLKVSVSKVMD
IFYQIRKADTSSRNVACDDPSSLDFLRKIQERSGLGDETYSPEGLIHVPP
RKTFAASREETEKVIIGALENLFENTKVNPREIGILVVNSSMFNPTPSLS
AMVVNTFKLRSNIKSFNLGGMGCSAGVIAIDLAKDLLHVHKNTYALVVST
ENITQGIYAGENRSMMVSNCLFRVGGAAILLSNKSGDRRRSKYKLVHTVR
THTGADDKSFRCVQQEDDESGKIGVCLSKDITNVAGTTLTKNIATLGPLI
LPLSEKFLFFATFVAKKLLKDKIKHYYVPDFKLAVDHFCIHAGGRAVIDE
LEKNLGLSPIDVEASRSTLHRFGNTSSSSIWYELAYIEAKGRMKKGNKAW
QIALGSGFKCNSAVWVALRNVKASANSPWQHCIDRYPVKIDSDLSKSKTH
VQNGRS

Yarrowia lipolytica YALIOB10153g (FAD2)
nucleotide sequence
(SEQ ID NO: 84)
atggattcgaccacgcagaccaacaccggcaccggcaaggtggcc
gtgcagccccccacggccttcattaagcccattgagaaggtgtcc
gagcccgtctacgacacctttggcaacgagttcactcctccagac
tactctatcaaggatattctggatgccattccccaggagtgctac
aagcggtcctacgttaagtcctactcgtacgtggcccgagactgc
ttctttatcgccgtttttgcctacatggcctacgcgtacctgcct
cttattccctcggcttccggccgagctgtggcctgggccatgtac
tccattgtccagggtctgtttggcaccggtctgtgggttcttgcc
cacgagtgtggccactctgctttctccgactctaacaccgtcaac
aacgtcaccggatgggttctgcactcctccatgctggtcccttac
tacgcctggaagctgacccactccatgcaccacaagtccactggt
cacctcacccgtgatatggtgtttgtgcccaaggaccgaaaggag
tttatggagaaccgaggcgcccatgactggtctgagcttgctgag
gacgctcccctcatgaccctctacggcctcatcacccagcaggtg
tttggatggcctctgtatctgctgtctaacgttaccggacagaag
taccccaagctcaacaaatgggctgtcaaccacttcaaccccaac
gccccgctgtttgagaagaaggactggttcaacatctggatctct
aacgtcggtattggtatcaccatgtccgtcatcgcatactccatc
aaccgatggggcctggcttccgtcaccctctactacctgatcccc
tacctgtgggtcaaccactggctcgtggccatcacctacctgcag
cacaccgaccccactctgccccactaccacgccgaccagtggaac
ttcacccgaggagccgccgccaccatcgaccgagagtttggcttc
atcggctccttctgcttccatgacatcatcgagacccacgttctg
caccactacgtgtctcgaattcccttctacaacgcccgaatcgcc
actgagaagatcaagaaggtcatgggcaagcactaccgacacgac
gacaccaacttcatcaagtctctttacactgtcgcccgaacctgc
cagtttgttgaaggtaaggaaggcattcagatgtttagaaacgtc
aatggagtcggagttgctcctgacggcctgccttctaaaaagtag

Yarrowia lipolytica YALIOB10153p (FAD2)
amino acid sequence
(SEQ ID NO: 85)
MDSTTQTNTGTGKVAVQPPTAFIKPIEKVSEPVYDTFGNEFTPPD
YSIKDILDAIPQECYKRSYVKSYSYVARDCFFIAVFAYMAYAYLP
LIPSASGRAVAWAMYSIVQGLFGTGLWVLAHECGHSAFSDSNTVN
NVTGWVLHSSMLVPYYAWKLTHSMHHKSTGHLTRDMVFVPKDRKE
FMENRGAHDWSELAEDAPLMTLYGLITQQVFGWPLYLLSNVTGQK
YPKLNKWAVNHFNPNAPLFEKKDWFNIWISNVGIGITMSVIAYSI
NRWGLASVTLYYLIPYLWVNHWLVAITYLQHTDPTLPHYHADQWN
FTRGAAATIDREFGFIGSFCFHDIIETHVLHHYVSRIPFYNARIA
TEKIKKVMGKHYRHDDTNFIKSLYTVARTCQFVEGKEGIQMFRNV
NGVGVAPDGLPSKK

Yarrowia lipolytica YALIOB20196p (FAE1/ELO2)
nucleotide sequence
(SEQ ID NO: 86)
atgagcgccgtccctattgaattcaacgtcccctccgtggaccga
ccctttggtatctacctctgggccatctttgaccaggcctgggag
aagcttttcggctggcccgcgtcctctttcattttcgtgcgaaat
gaccccaacatccccttttcctctacccctcccgtgatcattgcc
atcattgtgtactacattgtcatctttggcggccgagaggtgatg
cgaaacctgtctcccatccgactcaactggctcttccagatccac
aacatcttcctcacccttctgtccggtatgctcctcctcctcctc
gttgagcagctcttccccatcattgtccgacagggtatcctctac
gccatctgcgactacggatcttggactcagcccattgtcttctgc
tactacctcaactacctgaccaagtactttgagctgatcgacacc
gttttccttgtgctgcgaaagaagaagctgactttcctccacacc
taccaccatggtgccactgctcttctgtgctacacccagctcatt
ggtaagacctcggtctcttgggtccccatcacccttaacctgttt
gtccacgttgtcatgtacttctactacttcctggctgcgcgaggt
atccgagtgtggtggaaggagtgggtcacccggctccagatcatc
cagttcgttatcgatcttggatttgtctactttgcctcttacacc
tacttcacctctacctactggccctggatgcccaacatgggctct
tgtgccggcgaggagtttgctgctatttacggctgtggtctgctg
acctcttacctcttcctcttcatcgccttctacatcaactcttac
cgaaagccctcttccaagggaccttccaagcctgttgttgctgtc
gatggccctgttggcggcgtcaacgcccagactggtgcttctcga
ggccagaccactacccgatctcgacgagcataa

Yarrowia lipolytica YALIOB20196p (FAE1/ELO2)
amino acid sequence
(SEQ ID NO: 87)
MSAVPIEFNVPSVDRPFGIYLWAIFDQAWEKLFGWPASSFIFVRN
DPNIPFSSTPPVIIAIIVYYIVIFGGREVMRNLSPIRLNWLFQIH
NIFLTLLSGMLLLLLVEQLFPIIVRQGILYAICDYGSWTQPIVFC
YYLNYLTKYFELIDTVFLVLRKKKLTFLHTYHHGATALLCYTQLI
GKTSVSWVPITLNLFVHVVMYFYYFLAARGIRVWWKEWVTRLQII
QFVIDLGFVYFASYTYFTSTYWPWMPNMGSCAGEEFAAIYGCGLL
TSYLFLFIAFYINSYRKPSSKGPSKPVVAVDGPVGGVNAQTGASR
GQTTTRSRRA

Yarrowia lipolytica YALIOC10989g (CPT1)
nucleotide sequence
(SEQ ID NO: 88)
ATGGGCGTATTCATTAAACAGGAGCAGCTTCCGGCTCTCAAGAAG
TACAAGTACTCCGCCGAGGATCACTCGTTCATCTCCAACAACATT
CTGCGCCCCTTCTGGCGACAGTTTGTCAAAATCTTCCCTCTGTGG
ATGGCCCCCAACATGGTGACTCTGTTGGGCTTCTTCTTTGTCATT
GTGAACTTCATCACCATGCTCATTGTTGATCCCACCCACGACCGC
GAGCCTCCCAGATGGGTCTACCTCACCTACGCTCTGGGTCTGTTC
CTTTACCAGACATTTGATGCCTGTGACGGATCCCATGCCCGACGA
ACTGGCCAGAGTGGACCCCTTGGAGAGCTGTTTGACCACTGTGTC
GACGCCATGAATACCTCTCTGATTCTCACGGTGGTGGTGTCCACC
ACCCATATGGGATATAACATGAAGCTGCTGATTGTGCAGATTGCC
GCTCTCGGAAACTTCTACCTGTCGACCTGGGAGACCTACCATACC
GGAACTCTGTACCTTTCTGGCTTCTCTGGTCCTGTTGAAGGTATC
TTGATTCTGGTGGCTCTTTTCGTCCTCACCTTCTTCACTGGTCCC
AACGTGTACGCTCTGACCGTCTACGAGGCTCTTCCCGAATCCATC
ACTTCGCTGCTGCCTGCCAGCTTCCTGGACGTCACCATCACCCAG
ATCTACATTGGATTCGGAGTGCTGGGCATGGTGTTCAACATCTAC
GGCGCCTGCGGAAACGTGATCAAGTACTACAACAACAAGGGCAAG
AGCGCTCTCCCCGCCATTCTCGGAATCGCCCCCTTTGGCATCTTC
TACGTCGGCGTCTTTGCCTGGGCCCATGTTGCTCCTCTGCTTCTC
TCCAAGTACGCCATCGTCTATCTGTTTGCCATTGGGGCTGCCTTT
GCCATGCAAGTCGGCCAGATGATTCTTGCCCATCTCGTGCTTGCT
CCCTTCCCCCACTGGAACGTGCTGCTCTTCTTCCCCTTTGTGGGA
CTGGCAGTGCACTACATTGCACCCGTGTTTGGCTGGGACGCCGAT
ATCGTGTCGGTTAACACTCTCTTCACCTGTTTTGGCGCCACCCTC
TCCATTTACGCCTTCTTTGTGCTTGAGATCATCGACGAGATCACC
AACTACCTCGATATCTGGTGTCTGCGAATCAAGTACCCTCAGGAG
AAGAAGACTGAGTAA

Yarrowia lipolytica YALIOC10989p (CPT1)
amino acid sequence
(SEQ ID NO: 89)
MGVFIKQEQLPALKKYKYSAEDHSFISNNILRPFWRQFVKIFPLW
MAPNMVTLLGFFFVIVNFITMLIVDPTHDREPPRWVYLTYALGLF
LYQTFDACDGSHARRTGQSGPLGELFDHCVDAMNTSLILTVVVST
THMGYNMKLLIVQIAALGNFYLSTWETYHTGTLYLSGFSGPVEGI
LILVALFVLTFFTGPNVYALTVYEALPESITSLLPASFLDVTITQ
IYIGFGVLGMVFNIYGACGNVIKYYNNKGKSALPAILGIAPFGIF
YVGVFAWAHVAPLLLSKYAIVYLFAIGAAFAMQVGQMILAHLVLA
PFPHWNVLLFFPFVGLAVHYIAPVFGWDADIVSVNTLFTCFGATL
SIYAFFVLEIIDEITNYLDIWCLRIKYPQEKKTE

Yarrowia lipolytica YALI0E26565g (EPT1)
nucleotide sequence
(SEQ ID NO: 90)
ATGGGTAAAAGCCCCTCTATGATTGGGACTTGCGGTTGGGTTGGG
CTGGGTACAAACACATGCTATGCTCATCAGCTCAGCCGTGCCATG
GGCCATCAACCAACTTTTCTCCCGACCCTAATCCCCTCCACACTA
CTAATGCCAGGATTCCACCACATCAACCCCGACAGTCTAGTGCAT
CTAAAGTCGTACCAGTACAGAAGCGTCGATAAATCCTACCTGTCT
AAGTACATCCTGAACCCATGGTGGACTTACGCAGCAACCTTCATG
CCTGATTGGCTGGCTCCCAACGCCATCACTCTCATCGGTGTCTCC
GGCATGCTCCTGTCAATCTTCTTCACCGTCTGGTACACCCCCGAG
CTCACCGGCGACGGTCCCTCCTGGATCTACTTCTTCTCTGCCTTT
TCGCTCTTCTTCTACCAGACCATGGACAATATTGACGGCAAGCAG
GCCCGTCGAACTGGCTCTTCCTCTCCTCTGGGAGAGCTGTTTGAC
CACGGAATCGACTCCCTCAACTGCACCTACGGCGGAATTGTCAAC
TGCGGTGCTGTGGCCCTTGGTTCCACCTCCTACGGAGGCCTCATG
GTTCTGTCCACCTGTATTGGTATGTACTTTTCTACCTGGGAGACT
TACTACACCCACACTCTTTACCTGGGAGTTGTTAACGGCCCCACC
GAAGGGCTAGTGGTGGCCCTGTCTACCATGCTAATCTCCGGCTTC
ATGGGCACCGACATCTGGAAGGAGGATGCCGTTGAGGTGCTCCCC
TTCCTCTCCTTCATGGTTCCCGAGTACCTCAAGCTCAACGAGTTC
TGGGTCTGGGTCGTCATGTTCACCCTCGTGGTGCTGCACGTGCCT
TTCTGCGTGTGGAACGTCTATTGGGCCTGCAAGGAGGACGATGTG
CCTTTCTCCGAGGCTCTGGTGGGCCTCCTGCCCTTTGGCGTGGCC
GGAGGAGCTGCCTACGTGTGGCTGCAGAGTCCTTATTCCACTGTG
CTGGTTGACAACCACCTTGTGCTATTTGGACTGACCGCCTCGTGG
GTCTTTGGCCGGCTGACCACCGGTGTGATTCTCAACCACCTCACC
AAGCTCGAGTTCCCTCTGTGGAACTCCACCCTGATTCCTCTTCTG
GGAGCCACCGTTCTGTTCTACCTGCTCCCTGCCCTGGGTCTACTG
CCCCAGGACAATCCCCACTTCGAGACTCTGTATCTGTGGGGCTTC
TTTGTCTACGCTGCCGCGAACTTTTTGACCTGGGCTGTTAACACC
ATCAATGTCATCTGTTCCTACCTTGGCATCCGATGTCTGTCTCTG
CGACCCGTGGACAACAAGACCAACTAG

Yarrowia lipolytica YALI0E26565p (EPT1)
amino acid sequence
(SEQ ID NO: 91)
MGKSPSMIGTCGWVGLGTNTCYAHQLSRAMGHQPTFLPTLIPSTL
LMPGFHHINPDSLVHLKSYQYRSVDKSYLSKYILNPWWTYAATFM
PDWLAPNAITLIGVSGMLLSIFFTVWYTPELTGDGPSWIYFFSAF
SLFFYQTMDNIDGKQARRTGSSSPLGELFDHGIDSLNCTYGGIVN
CGAVALGSTSYGGLMVLSTCIGMYFSTWETYYTHTLYLGVVNGPT
EGLVVALSTMLISGFMGTDIWKEDAVEVLPFLSFMVPEYLKLNEF
WVWVVMFTLVVLHVPFCVWNVYWACKEDDVPFSEALVGLLPFGVA
GGAAYVWLQSPYSTVLVDNHLVLFGLTASWVFGRLTTGVILNHLT
KLEFPLWNSTLIPLLGATVLFYLLPALGLLPQDNPHFETLYLWGF
FVYAAANFLTWAVNTINVICSYLGIRCLSLRPVDNKTN

### REFERENCES

1) Alonso JM et al., (2003) Genome-wide insertional mutagenesis of Arabidopsis thaliana. Science 301:653-657.
2) Lu C, Xin Z, Ren Z, Miquel M, Browse J (2009) An enzyme regulating triacylglycerol composition is encoded by the ROD1 gene of Arabidopsis. Proc Natl Acad Sci USA 106:18837-18842.
3) Zhang C et al., (2013) Genetic and biochemical basis for alternative routes of tocotrienol biosynthesis for enhanced vitamin E antioxidant production. Plant J 73:628-639.
4) Clough SJ, Bent AF (1998) Floral dip: a simplified method for Agrobacterium-mediated transformation of Arabidopsis thaliana. Plant J 16:735-743.
5) Wood CC et al., (2009) A leaf-based assay using interchangeable design principles to rapidly assemble multistep recombinant pathways. Plant Biotech J 7:914-924.
6) Kelly AA et al., (2013) The SUGAR-DEPENDENT1 lipase limits triacylglycerol accumulation in vegetative tissues of Arabidopsis. Plant Physiol. 162:1282-1289.
7) van Erp H, Bates PD, Burgal J, Shockey J, Browse J (2011) Castor phospholipid: diacylglycerol acyltransferase facilitates efficient metabolism of hydroxy fatty acids in transgenic Arabidopsis. Plant Physiol. 155:683-693.
8) Bates PD, Browse J (2011) The pathway of triacylglycerol synthesis through phosphatidylcholine in Arabidopsis produces a bottleneck for the accumulation of unusual fatty acids in transgenic seeds. Plant J. 68:387-399.
9) van Erp H, Kelly AA, Menard G, Eastmond PJ (2014) Multigene engineering of triacylglycerol metabolism boosts seed oil content in Arabidopsis. Plant Physiol. 165:30-36.
10) Yamada T et al., (2013) Development of a lipid profiling system using reverse-phase liquid chromatography coupled to high-resolution mass spectrometry with rapid polarity switching and an automated lipid identification software. J Chrom. A 1292:211-218.
11) Narvaez-Rivas M, Zhang G (2016) Comprehensive untargeted lipidomic analysis using core-shell C30 particle column and high field orbitrap mass spectrometer. J Chrom. A 1440:123-134.
12) Turner N et al., (2018) A selective inhibitor of ceramide synthase 1 reveals a novel role in fat metabolism, Nature Comm. 9:3165.
13) Mendes A et al., (2013) bZIP67 regulates the omega-3 fatty acid content of Arabidopsis seed oil by activating FATTY ACID DESATURASE 3. Plant Cell 25:3104-3116.
14) Gutierrez L et al., (2008) The lack of a systematic validation of reference genes: a serious pitfall undervalued in reverse transcription-polymerase chain reaction (RT-PCR) analysis in plants. Plant Biotechnol J. 6:609-618.
15) Nguyen HT, Mishra G, Whittle E, Pidkowich MS, Bevan SA, Merlo AO, Walsh TA, Shanklin J (2010) Metabolic engineering of seeds can achieve levels of omega-7 fatty acids comparable with the highest levels found in natural plant sources. Plant Physiol. 154:1897-1904.
16) Ossowski S, Schwab R, Weigel D (2008) Gene silencing in plants using artificial microRNAs and other small RNAs. Plant J. 53:674-690.
17) Nguyen HT, Park H, Koster KL, Cahoon RE, Nguyen HT, Shanklin J, Clemente TE, Cahoon EB (2015) Redirection of metabolic flux for high levels of omega-7 monounsaturated fatty acid accumulation in camelina seeds. Plant Biotechnol J. 13:38-50.
18) Moloney, M.M., Walker, J.M. and Sharma, K.K. (1989) High efficiency transformation of Brassica napus using agrobacterium vectors. Plant Cell Rep., 8, 238-242.
19) Barth G, Gaillardin C. 1996. Yarrowia lipolytica. In: . Nonconv. Yeasts Biotechnol. SE—10. Springer Berlin Heidelberg, pp. 313-388.
20) Sambrook J, Russell DW. 2001. Molecular Cloning: A Laboratory Manual, 3rd edn. Cold Spring Harbour, NY, USA Cold Spring Harb. Lab. Press. 2001. Vol. 1-3.
21) Gibson DG, Young L, Chuang R-Y, Venter JC, Hutchison CA, Smith HO. 2009. Enzymatic assembly of DNA molecules up to several hundred kilobases. Nat. Methods 6:343-345.
22) Kulasekara HD. 2011. Gibson Assembly. Samuel Mill. Lab, UW, Seattle. http://miller-lab.net/MillerLab/protocols/molecular-biology-and-cloning/gibson-assembly/.
23) Bhutada G, Kavšček M, Ledesma-Amaro R, Thomas S, Rechberger GN, Nicaud J-M, Natter K. 2017. Sugar versus fat: Elimination of glycogen storage improves lipid accumulation in Yarrowia lipolytica. FEMS Yeast Res. 17.
24) Curran KA, Morse NJ, Markham KA, Wagman AM, Gupta A, Alper HS. 2015. Short, Synthetic Terminators for Improved Heterologous Gene Expression in Yeast. ACS Synth. Biol.
25) Le Dall MT, Nicaud JM, Gaillardin C. 1994. Multiple-copy integration in the yeast Yarrowia lipolytica. Curr. Genet. 26:38-44.
26) Wu J, James DW Jr, Dooner HK, Browse J (1994) A Mutant of Arabidopsis Deficient in the Elongation of Palmitic Acid. Plant Physiol 106:143-150
27) Smith MA, Moon H, Chowrira G, Kunst L (2003) Heterologous expression of a fatty acid hydroxylase gene in developing seeds of Arabidopsis thaliana. Planta 217:507-516
28) Kim HU, Li Y, Huang AH (2005) Ubiquitous and endoplasmic reticulum-located lysophosphatidyl acyltransferase, LPAT2, is essential for female but not male gametophyte development in Arabidopsis. Plant Cell 17:1073-1089

As described herein, in human milk fat, saturated fatty acids are esterified to the middle position on the glycerol backbone giving the triacylglycerol molecules an unusual stereochemistry that assists nutrient absorption in the infant gut. However, the fat used in most infant formulas is derived from plants, which esterify saturated fatty acids to the outer positions. Here we have engineered the metabolism of an oilseed plant so that it accumulates triacylglycerol with more than 70% of the saturated fatty acid palmitate in the middle position, thereby mimicking human milk fat stereoisomeric structure. Applying this technology to oilseed crops (or oleaginous microbes) could provide a new source of human milk fat substitute for infant nutrition.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

The content of all references cited herein is incorporated herein by reference in its entirety.

What is claimed herein:

1. An algal organism for producing triacylglycerol
(A) wherein the algal organism is genetically modified to express a lysophosphatidic acid acyltransferase specific for C16:0-Coenzyme A (C16:0 LPAT) and wherein, once expressed, the C16:0 LPAT is localised in the endoplasmic reticulum; or
(B) in which:
(a) a lysophosphatidic acid acyltransferase specific for C16:0-Coenzyme A (C16:0 LPAT) is localised in the endoplasmic reticulum; and
(b) activity of native endoplasmic reticulum lysophosphatidic acid acyltransferase (ER LPAT) is suppressed or prevented.

2. The algal organism according to claim 1, wherein the activity of diacylglycerol conversion to and from phosphatidylcholine is suppressed or prevented.

3. The algal organism according to claim 1, wherein the activity of phosphatidylcholine:diacylglycerol cholinephosphotransferase (PDCT) is suppressed or prevented.

4. The algal organism according to claim 1, wherein the organism is a Prototheca algae.

5. The algal organism according to claim 1, wherein the algal organism is cultured in a media comprising a carbon source, wherein the carbon source comprises (i) one or more sugars, and (ii) one or more fatty acids and/or fatty acid esters.

6. The algal organism according to claim 5, wherein the carbon source comprises a mixture of fatty acids and/or fatty acid esters wherein at least about 30% w/w of the fatty acids and/or fatty acid esters comprises C16:0 and/or at least about 30% w/w of the fatty acids and/or fatty acid esters comprises C18:1.

7. The algal organism according to claim 5, wherein the media comprises glycerol.

8. The algal organism according to claim 1, wherein the organism is cultured in a media comprising a carbon source, wherein the carbon source comprises palm oil.

9. The algal organism according to claim 8, wherein the carbon source comprises palm oil in combination with glucose and/or glycerol.

10. The algal organism according to claim 1, wherein the C16:0 LPAT is selected from:
(i) a chloroplast LPAT which lacks a functional chloroplast targeting sequence;
(ii) AGPAT1 (Human 1-acylglycerol-3-phosphate O-acyltransferase isoform 1);
(iii) CreLPAT;
(iv) Nannochloropsis sp. LPAT2;
(v) Nannochloropsis sp. LPAT3;
(vi) Nannochloropsis sp. LPAT4; and
(vii) Synechocystis sp. LPAT.

11. The algal organism according to claim 1, wherein the algal organism expresses a FATB thioesterase.

12. The algal organism according to claim 1, wherein the algal organism expresses a FATB thioesterase and is treated or genetically modified to disrupt the fatty acid desaturase 2 gene (FAD2).

13. The algal organism according to claim 1, wherein the algal organism is treated or genetically modified to disrupt the KASII gene FAB1, fatty acid elongase gene (FAE1), and/or fatty acid desaturase 2 gene (FAD2).

14. The algal organism according to claim 1, wherein
(i) the algal organism expresses a FATB thioesterase and is treated or genetically modified to disrupt the fatty acid desaturase 2 gene (FAD2),
(ii) conversion of diacylglycerol to phosphatidylcholine is suppressed or prevented in the algal organism, and
(iii) activity of native endoplasmic reticulum lysophosphatidic acid acyltransferase (ER LPAT) is suppressed or prevented in the algal organism.

15. A cell of the algal organism according to claim 1.

16. Triacylglycerol produced from the algal organism or a cell thereof according to claim 1.

17. The triacylglycerol according to claim 16, wherein the triacylglycerol comprises more than about 30% of the C16:0 at the sn-2 position.

18. The triacylglycerol according to claim 16, wherein the triacylglycerol comprises more than about 30% of the C16:0 at the sn-2 position.

19. A method for extracting triacylglycerol from the algal organism according to claim 1.

20. A method for producing an infant formula, comprising obtaining triacylglycerol from an algal organism or a cell thereof according to claim 1 and using said triacylglycerol to produce an infant formula.

21. Infant formula comprising triacylglycerol according to claim 16.
